(19) United States
(12) Patent Application Publication
(10) Pub. No.: US 2004/0138274 A1
(43) Pub. Date: Jul. 15, 2004

(54) Novel pharmaceuticals

(76) Inventors: Kevin Neil Dack, Sandwich (GB); Dafydd Rhys Owen, Sandwich (GB); Christine Anne Louise Watson, Sandwich (GB)

Correspondence Address: WARNER-LAMBERT COMPANY, 2800 PLYMOUTH RD, ANN ARBOR, MI, 48105, US

(21) Appl. No.: 10/739,426

(22) Filed: Dec. 18, 2003

Related U.S. Application Data
(60) Provisional application No. 60/448,224, filed on Feb. 18, 2003.

(30) Foreign Application Priority Data
Dec. 23, 2002 (GB) 0230025.9

(51) Int. Cl. C07D 213/55; A61K 31/44; A61K 31/40

(52) U.S. Cl. 514/357; 514/408; 546/335; 548/571

(57) ABSTRACT

The invention relates to NEP inhibitors for treating cardiovascular disorders wherein R¹ is C₁-C₆alkyl, C₁-C₆alkoxyC₁-C₃alkyl or C₁-C₆alkoxyC₁-C₆alkoxyC₁-C₃alkyl; R² is hydrogen or C₁-C₆alkyl; L is an aromatic heterocyclic ring, optionally substituted with C₁-C₆alkyl or halo; R³ is C₁-C₆alkyl optionally substituted by halo, alkoxy, haloalkoxy, alkylthio, haloalkylthio or nitrile group, or R³ is phenyl or aromatic heterocyclyl each of which may be independently substituted by one or more alkyl, halo, haloalkyl, alkoxy, haloalkoxy, alkylthio, haloalkylthio or nitrile group; R⁴ and R⁵ are either both hydrogen, or one of R⁴ and R⁵ is hydrogen and the other is a biolabile ester-forming group that in the body of a patient is replaced by hydrogen; p is 0, 1 or 2; and q is 1 or 2.

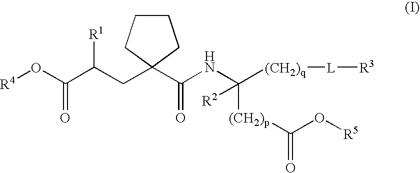

This application claims the benefit of foreign priority under 35 U.S.C. section 119(a) to United Kingdom provisional patent application No. 0230025.9, filed on Dec. 23, 2002. The present application also claims priority under 35 U.S.C. section 119(e) to U.S. provisional application No. 60/448,244, filed Feb. 18, 2003.

The invention relates to a series of cyclopentyl substituted glutaramide derivatives and compositions and uses thereof. The derivatives are potent and selective inhibitors of neutral endopeptidase (NEP) and may be used to treat a number of diseases and conditions particularly cardiovascular disorders, especially hypertension.

According to a first aspect, the invention provides a compound of formula (I), a pharmaceutically acceptable salt or solvate thereof

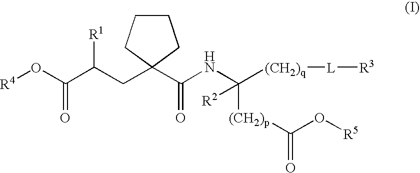

wherein

R¹ is C₁-C₆alkyl, C₁-C₆alkoxyC₁-C₃alkyl or C₁-C₆alkoxyC₁-C₆alkoxyC₁-C₃alkyl;

R² is hydrogen or C₁-C₆alkyl;

L is an aromatic heterocyclic ring, optionally substituted with C₁-C₆alkyl or halo;

R³ is C₁-C₆alkyl optionally substituted by halo, alkoxy, haloalkoxy, alkylthio, haloalkylthio or nitrile group, or R³ is phenyl or aromatic heterocyclyl each of which may be independently substituted by one or more alkyl, halo, haloalkyl, alkoxy, haloalkoxy, alkylthio, haloalkylthio or nitrile group;

R⁴ and R⁵ are either both hydrogen, or one of R⁴ and R⁵ is hydrogen and the other is a biolabile ester-forming group that in the body of a patient is replaced by hydrogen;

p is 0, 1 or 2; and

q is 1 or 2.

Unless otherwise indicated, any alkyl group may be straight or branched and is of 1 to 6 carbon atoms, preferably 1 to 4.

Unless otherwise indicated, any carbocyclyl group contains 3 to 8 ring-atoms, and may be saturated, unsaturated or aromatic. Preferred saturated carbocyclyl groups are cyclopropyl, cyclobutyl, cyclopentyl or cyclohexyl. Preferred unsaturated carbocyclyl groups contain up to 3 double bonds. A preferred aromatic carbocyclyl group is phenyl. The term carbocylic should be similarly construed. In addition, the term carbocyclyl includes any fused combination of carbocyclyl groups, for example naphthyl, phenanthryl, indanyl and indenyl.

Unless otherwise indicated, any heterocyclyl group contains 5 to 7 ring-atoms up to 4 of which may be hetero-atoms such as nitrogen, oxygen and sulfur, and may be saturated, unsaturated or aromatic. Examples of heterocyclyl groups are furyl, thienyl, pyrrolyl, pyrrolinyl, pyrrolidinyl, imidazolyl, dioxolanyl, oxazolyl, thiazolyl, imidazolyl, imidazolinyl, imidazolidinyl, pyrazolyl, pyrazolinyl, pyrazolidinyl, isoxazolyl, isothiazolyl, oxadiazolyl, triazolyl, thiadiazolyl, pyranyl, pyridyl, piperidinyl, dioxanyl, morpholino, dithianyl, thiomorpholino, pyridazinyl, pyrimidinyl, pyrazinyl, piperazinyl, sulfolanyl, tetrazolyl, triazinyl, azepinyl, oxazepinyl, thiazepinyl, diazepinyl and thiazolinyl. In addition, the term heterocyclyl includes fused heterocyclyl groups, for example benzimidazolyl, benzoxazolyl, imidazopyridinyl, benzoxazinyl, benzothiazinyl, oxazolopyridinyl, benzofuranyl, quinolinyl, quinazolinyl, quinoxalinyl, dihydroquinazolinyl, benzothiazolyl, phthalimido, benzofuranyl, benzodiazepinyl, indolyl and isoindolyl. The term heterocyclic should be similarly construed.

Halo means fluoro, chloro, bromo or iodo.

Unless otherwise indicated, any haloalkyl, haloalkoxy or haloalkylthio group contains one or more halo atoms which halo atoms may be the same or different.

Preferably R¹ is C₁-C₆alkyl or C₁-C₆alkoxyC₁-C₃alkyl. More preferably R¹ is propyl or methoxyethyl.

Preferably R² is hydrogen.

Preferably L is a non-fused aromatic heterocyclic ring optionally substituted by C₁-C₆alkyl. More preferably a five membered aromatic heterocyclic ring. More preferably oxazole, oxadiazole, imidazole or pyrazole. More preferably still oxazole or oxadiazole.

Preferably R³ is C₁-C₆alkyl or R³ is phenyl which may be independently substituted by one or more C₁-C₆alkyl, halo, haloC₁-C₆alkyl, C₁-C₆alkoxy, haloalkoxy, C₁-C₆alkylthio, haloC₁-C₆alkylthio or nitrile group. More preferably R³ is phenyl optionally substituted by halo. More preferably still R³ is phenyl, 4-fluorophenyl, or 4-chlorophenyl.

The term biolabile ester-forming group is well understood in the art as meaning a group which provides an ester that can be readily cleaved in the body to liberate the corresponding diacid of formula (I) wherein R⁴ and R⁵ are both hydrogen. A number of such ester groups are well known, for example in the penicillin area or in the case of the ACE inhibitor hypertensives such as enalapril and quinapril. Compounds of formula (I) containing such biolabile ester-forming groups are particularly advantageous in providing compounds suitable for oral administration. The suitability of any particular ester-forming group can be assessed by conventional animal or in vitro enzyme hydrolysis studies. For optimum effect, the ester should only be hydrolysed after absorption. Accordingly the ester should be resistant to hydrolysis before absorption by digestive enzymes but should be readily hydrolysed by, for example, liver or plasma enzymes. In this way the active diacid is released into the blood stream following oral absorption.

Preferred biolabile ester-forming groups are C₁-C₆alkyl, carbocyclyl, heterocyclyl or carbocyclylalkyl each of which may be substituted.

More preferred biolabile ester-forming groups are: i) C₁-C₆alkyl optionally substituted by hydroxy, oxo, halo, haloC₁-C₆alkyl, C₁-C₆alkoxy, haloC₁-C₆alkoxy, C₁-C₆alkylthio, haloC₁-C₆alkylthio, nitrile, carbocyclyl, heterocyclyl, carbocyclyloxy, heterocyclyloxy, C₁-C₇alkylcarbonyloxy, carbocyclylcarbonyloxy, heterocyclylcarbonyloxy, alkylcarbonylamino, and alkylaminocarbonyl, wherein any carbocyclyl or heterocyclyl group is optionally substituted by C₁-C₆alkyl, halo, haloC₁-C₆alkyl, C₁-C₆alkoxy, haloC₁-C₆alkoxy, C₁-C₆alkylthio, haloC₁-C₆alkylthio or nitrile; or ii) carbocyclyl or heterocyclyl optionally substituted by C₁-C₆alkyl, halo, haloC₁-C₆alkyl, C₁-C₆alkoxy, haloC₁-C₆alkoxy, C₁-C₆alkylthio, haloC₁-C₆alkylthio or nitrile. In the definitions of i) and ii) any carbocyclic group is preferably phenyl and any heterocyclic group is aromatic.

Still more preferred biolabile ester-forming groups are selected from the list: ethyl, propyl, butyl, isobutyl, cyclopentyl, benzyl, 1-(2,2-diethylbutyryloxy)ethyl, 2-ethylpropionyloxymethyl, 1-(2-ethylpropionyloxy)ethyl, 1-(2,4-dimethylbenzoyloxy)ethyl, 1-benzoyloxy)benzyl, 1-(benzoyloxy)ethyl, 2-methyl-1-propionyloxypropyl, 2,4,6-trimethylbenzoyloxymethyl, 1-(2,4,6-trimethylbenzyloxy)ethyl, pivaloyloxymethyl, phenethyl, phenpropyl, 2,2,2-trifluororethyl, 1-naphthyl, 2-naphthyl, 2,4-dimethylphenyl, 4-t-butylphenyl, 5-(4-methyl-1,3-dioxalynyl-2-onyl)methyl, N,N-diethylaminocarbonylmethyl and 5-indanyl.

Preferably R⁴ and R⁵ are both hydrogen.

Preferably p is 0 or 1. More preferably p is 0.

Preferably q is 1.

A preferred compound is of formula (I′) wherein R¹, R², R³, R⁴, R⁵, L, p and q are as defined in the first aspect.

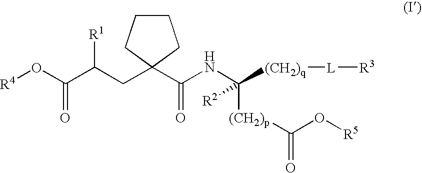

A preferred compound of formula (I′) is where

R¹ is C₁-C₆alkyl or C₁-C₆alkoxyC₁-C₃alkyl;

R² is hydrogen;

L is a non-fused five membered aromatic heterocyclic ring optionally substituted by C₁-C₆alkyl;

R³ is C₁-C₆alkyl or R³ is phenyl which may be independently substituted by one or more C₁-C₆alkyl, halo, haloC₁-C₆alkyl, C₁-C₆alkoxy, haloalkoxy, C₁-C₆alkylthio, haloC₁-C₆alkylthio or nitrile group;

R⁴ and R⁵ are either both hydrogen, or one of R⁴ and R⁵ is hydrogen and the other is a biolabile ester-forming group selected from the list: i) C₁-C₆alkyl optionally substituted by hydroxy, oxo, halo, haloC₁-C₆alkyl, C₁-C₆alkoxy, haloC₁-C₆alkoxy, C₁-C₆alkylthio, haloC₁-C₆alkylthio, nitrile, carbocyclyl, heterocyclyl, carbocyclyloxy, heterocyclyloxy, alkylcarbonyloxy, carbocyclylcarbonyloxy, heterocyclylcarbonyloxy, alkylcarbonylamino, and alkylaminocarbonyl, wherein any carbocyclyl or heterocyclyl group is optionally substituted by C₁-C₆alkyl, halo, haloC₁-C₆alkyl, C₁-C₆alkoxy, haloC₁-C₆alkoxy, C₁-C₆alkylthio, haloC₁-C₆alkylthio or nitrile; or ii) carbocyclyl or heterocyclyl optionally substituted by C₁-C₆alkyl, halo, haloC₁-C₆alkyl, C₁-C₆alkoxy, haloC₁-C₆alkoxy, C₁-C₆alkylthio, haloC₁-C₆alkylthio or nitrile;

p is 0 or 1; and

q is 1.

A more preferred compound of formula (I′) is where

R¹ is propyl or methoxyethyl;

R² is hydrogen;

L is oxazole, oxadiazole, imidazole or pyrazole each of which may be substituted by C₁-C₆alkyl;

R³ is phenyl, 4-fluorophenyl, or 4-chlorophenyl;

R⁴ and R⁵ are both hydrogen;

p is 0; and

q is 1.

A particularly preferred compound of formula (I) is selected from:

(2S)-2-{1-[(1S)-1-Carboxy-2-(4-methyl-5-phenyl-oxazol-2-yl)-ethoxycarbamoyl]-cyclopentylmethyl}-4-methoxy-butyric acid (Example 25);

(2S)-2-(1-{(1S)-1-Carboxy-2-[5-(4-fluoro-phenyl)-oxazol-2-yl]-ethylcarbamoyl}-cyclopentylmethyl-4-methoxy-butyric acid (Example 26);

(2R)-2-{1-[(1S)-1-Carboxy-2-(5-phenyl-oxazol-2-yl)-ethylcarbamoyl]-cyclopentylmethyl}-pentanoic acid (Example 29);

(2S)-2-(1-{(1S)-1-Carboxy-2-[5-(4-chloro-phenyl)-oxazol-2-yl]-ethylcarbamoyl}-cyclopentylmethyl)-4-methoxy-butyric acid (Example 30);

(2S)-2-{1-[(1S)-1-Carboxy-2-(5-phenyl-[1.2.4]oxadiazol-3-yl)-ethylcarbamoyl]-cyclopentlymethyl}-4-methoxy-butyric acid (Example 35);

(2R)-2-{1-[(1S)-1-carboxy-2-(4-phenyl-pyrazol-1-yl)-ethylcarbamoyl]-cyclopentylmethyl}-pentanoic acid (Example 36); and

(2S)-2-{1-[(1S)-1-Carboxy-2-(5-phenyl-oxazol-2-yl)-ethylcarbamoyl]-cyclopentylmethyl}-4-methoxy-butyric acid (Example 40).

For the avoidance of doubt, unless otherwise indicated, the term substituted means substituted by one or more defined groups. In the case where groups may be selected from a number of alternative groups, the selected groups may be the same or different.

For the avoidance of doubt, the term independently means that where more than one substituent is selected from a number of possible substituents, those substituents may be the same or different.

The pharmaceutically or veterinarily acceptable salts of the compounds of the invention which contain a basic centre are, for example, non-toxic acid addition salts formed with inorganic acids such as hydrochloric, hydrobromic, hydroiodic, sulfuric and phosphoric acid, with carboxylic acids or with organo-sulfonic acids. Examples include the HCl, HBr, HI, sulfate or bisulfate, nitrate, phosphate or hydrogen phosphate, acetate, benzoate, succinate, saccharate, fumarate, maleate, lactate, citrate, tartrate, gluconate, camsylate, methanesulfonate, ethanesulfonate, benzenesulfonate, p-toluenesulfonate and pamoate salts. Compounds of the invention can also provide pharmaceutically or veterinarily acceptable metal salts, in particular non-toxic alkali and alkaline earth metal salts, with bases. Examples include the sodium, potassium, aluminium, calcium, magnesium, zinc and diethanolamine salts. For reviews on suitable pharmaceutical salts see Berge et al., J. Pharm, Sci., 66, 1-19, 1977; P L Gould, International Journal of Pharmaceutics, 33 (1986), 201-217; and Bighley et al, Encyclopedia of Pharmaceutical Technology, Marcel Dekker Inc, New York 1996, Volume 13, page 453-497.

The pharmaceutically acceptable solvates of the compounds of the invention include the hydrates thereof.

Also included within the scope of the invention and various salts of the invention are polymorphs thereof.

Hereinafter, compounds their pharmaceutically acceptable salts, their solvates or polymorphs, defined in any aspect of the invention (except intermediate compounds in chemical processes) are referred to as “compounds of the invention”.

The compounds of the invention may possess one or more chiral centres and so exist in a number of stereoisomeric forms. All stereoisomers and mixtures thereof are included in the scope of the present invention. Racemic compounds may either be separated using preparative HPLC and a column with a chiral stationary phase or resolved to yield individual enantiomers utilising methods known to those skilled in the art. In addition, chiral intermediate compounds may be resolved and used to prepare chiral compounds of the invention.

The compounds of the invention may exist in one or more tautomeric forms. All tautomers and mixtures thereof are included in the scope of the present invention. For example, a claim to 2-hydroxypyridinyl would also cover its tautomeric form, α-pyridonyl.

The invention also includes all suitable isotopic variations of a compound of the invention. An isotopic variation of a compound of the invention is defined as one in which at least one atom is replaced by an atom having the same atomic number but an atomic mass different from the atomic mass usually found in nature. Examples of isotopes that can be incorporated into compounds of the invention include isotopes of hydrogen, carbon, nitrogen, oxygen, phosphorus, sulphur, fluorine and chlorine such as ²H, ³H, ¹³C, ¹⁴C, ¹⁵N, ¹⁷O, ¹⁸O, ³²P, ³⁵S, ¹⁸F and ³⁶Cl, respectively. Certain isotopic variations of the invention, for example, those in which a radioactive isotope such as ³H or ¹⁴C is incorporated, are useful in drug and/or substrate tissue distribution studies. Tritiated, i.e., ³H, and carbon-14, i.e., ¹⁴C, isotopes are particularly preferred for their ease of preparation and detectability. Further, substitution with isotopes such as deuterium, i.e., ²H, may afford certain therapeutic advantages resulting from greater metabolic stability, for example, increased in vivo half-life or reduced dosage requirements and hence may be preferred in some circumstances. Isotopic variations of the compounds of the invention can generally be prepared by conventional procedures such as by the illustrative methods or by the preparations described in the Examples and Preparations hereafter using appropriate isotopic variations of suitable reagents.

The compounds of the invention, by inhibiting NEP (particularly EC.3.4.24.11), can potentiate the biological effects of bioactive peptides and thus there is rationale for the compounds of the invention to treat or prevent a number of diseases and conditions.

Shepperson et al have demonstrated that the NEP inhibitor candoxatrilat lowers systolic blood pressure in hypertensive rats [see Clin. Sci (Lond), Vol 80(3):265-9]. Kosoglou et al have demonstrated that the NEP inhibitor SCH34826 significantly lowered supine blood pressure in a clinical study of 24 black patients with essential hypertension [Circulation, Supplement III, Vol 82, No. 4, page 554, 2201]. Stergiou et al have demonstrated that when added to the ACE inhibitor lisinopril, candoxatril (the oral prodrug of candoxatrilat) led to a marked reduction in both supine and erect blood pressure in a clinical study involving 37 hypertensive patients [J. Hypertens, Vol 12, No 11, page 1310-1311]. Accordingly, the compounds of the invention should treat or prevent cardiovascular diseases and conditions, particularly hypertension, pulmonary hypertension, peripheral vascular disease, heart failure, angina, renal insufficiency, acute renal failure, cyclical oedema, Menières disease, hyperaldosteroneism (primary and secondary) and hypercalciuria. The term hypertension includes all diseases characterised by supranormal blood pressure, such as essential hypertension, pulmonary hypertension, secondary hypertension, isolated systolic hypertension, hypertension associated with diabetes, hypertension associated with atherosclerosis, and renovascular hypertension, and further extends to conditions for which elevated blood pressure is a known risk factor. Accordingly, the term “treatment of hypertension” includes the treatment or prevention of complications arising from hypertension, and other associated co-morbidities, including congestive heart failure, angina, stroke, glaucoma, impaired renal function, including renal failure, obesity, and metabolic diseases (including Metabolic Syndrome). Metabolic diseases include in particular diabetes and impaired glucose tolerance, including complications thereof, such as diabetic retinopathy and diabetic neuropathy.

The compounds of the invention should be particularly efficacious in treating or preventing hypertension.

Wayman et al [WO 02/079143] have demonstrated that NEP inhibitors increase vaginal and clitoral blood flow in a rabbit model of female sexual arousal disorder (FSAD). Accordingly the compounds of the invention should treat or prevent female sexual dysfunction, particularly FSAD.

Wayman et al (WO 02/03995) have demonstrated that NEP inhibitors potentiate nerve-stimulated erections in anaesthetised dog model of penile erections.

Accordingly the compounds of the invention should treat or prevent male erectile dysfunction (MED).

As a result of their ability to inhibit NEP, the compounds of the invention should treat or prevent menstrual disorders, pre-term labour, pre-eclampsia, endometriosis, and reproductive disorders (especially male and female infertility, polycystic ovarian syndrome, implantation failure).

In addition, the compounds of the invention should: treat or prevent asthma, inflammation, leukemia, pain, epilepsy, affective disorders, dementia and geriatric confusion, septic shock, obesity and gastrointestinal disorders (especially diarrhoea and irritable bowel syndrome); promote wound healing (especially diabetic and venous ulcers and pressure sores); modulate gastric acid secretion; and treat of hyperreninaemia, cystic fibrosis, restenosis, diabetic complications and atherosclerosis.

As used herein, the terms “treat”, “treating” and ‘treatment’ include palliative, curative and prophylactic treatment.

Compounds of the invention may be prepared, in known manner in a variety of ways. In the following reaction schemes and hereafter, unless otherwise stated, R¹ to R⁵, L, p and q are as defined in the first aspect. These processes form further aspects of the invention.

Throughout the specification, general formulae are designated by Roman numerals I, II, III, IV etc. Subsets of these general formulae are defined as Ia, Ib, Ic etc, . . . IVa, IVb, IVc etc.

Compounds of formula (Ia), i.e. compounds of general formula I where R⁴ and R⁵ are hydrogen, may be prepared according to reaction scheme 1, by deprotecting a compound of general formula (II) where P¹ and P², which may be the same or different, are suitable carboxyl protecting groups, such as C₁-C₄ alkyl, allyl or benzyl, preferably ethyl or t-butyl.

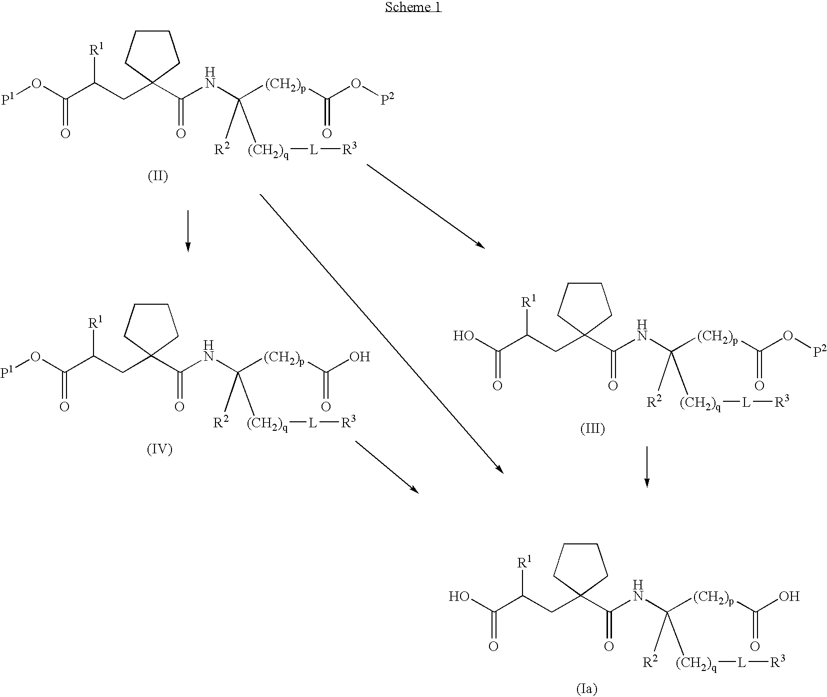

Deprotection can be performed using standard methodology, as described in “Protecting Groups in Organic Synthesis” by T. W. Greene and P. Wutz. When P¹ or P² is t-butyl, then the preferred conditions are 9-40% trifluoroacetic acid in dichloromethane (by volume) at room temperature for 1 to 72 hours. When P¹ or P² is allyl, preferred conditions are pyrrolidine (4 equiv), tetrakistriphenylphosphine paladium (0) (catalytic) in tetrahydrofuran at room temperature for 2-3 hours. When P¹ or P² is ethyl, preferred conditions are 1N sodium hydroxide in dioxan, methanol or tetrahydrofuran at a temperature between room temperature and reflux for 2-18 hours. When P¹ or P² is benzyl, preferred conditions are hydrogenolysis in an alcoholic solvent such as methanol using hydrogen gas at atmospheric or elevated pressure or another suitable hydrogen source, such as ammonium formate, in the presence of a suitable catalyst, such as palladium-on-carbon or palladium hydroxide, at a temperature between room temperature and reflux for 2-18 hours.

The deprotection of the two carboxyl groups may be carried out simultaneously or sequentially, via intermediate compounds of formulae (III) or (IV). These intermediate compounds may optionally be isolated and purified.

When P¹ and P² are the same then the deprotection of both carboxyl groups will generally be carried out simultaneously, allowing the compounds of formula (Ia) to be prepared directly from the compounds of formula (II). When P¹ and P² are different then, depending on the nature of P¹ and P² and the conditions chosen for the deprotection reaction, the two carboxyl groups may be deprotected sequentially or simultaneously.

Compounds of formula (Ib), i.e. compounds of formula (I) wherein R⁴ is a biolabile group and R⁵ is hydrogen, may be prepared from compounds of formula (III) according to reaction scheme 2.

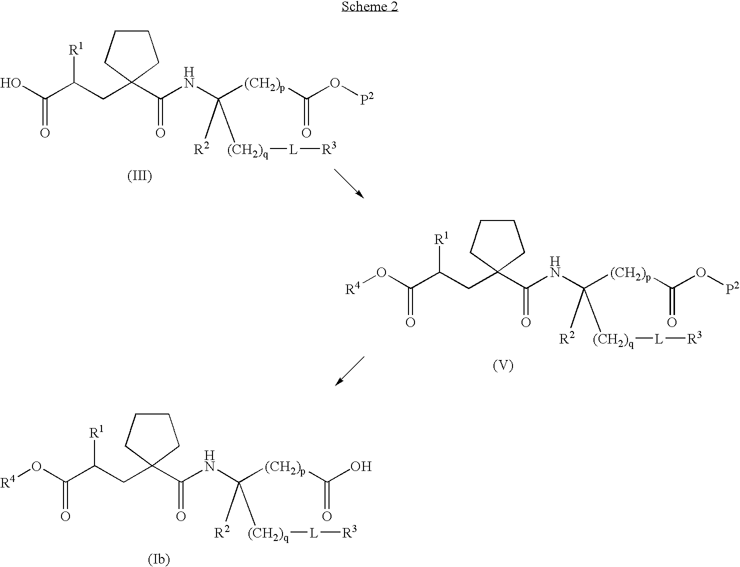

Compounds of formula (V) may be prepared by treating compounds of formula (III) with an alcohol R⁴—OH in the presence of a dehydrating agent such as carbonyldiimidazole or dicyclohexylcarbodiimide and optionally a catalyst such as pyridine, dimethylaminopyridine or triethylamine, in a solvent such as dichloromethane or dimethylformamide at a temperature between 0° C. and the reflux temperature of the solvent for 1-24 hours. Alternatively, compounds of formula (V) may be prepared by treating compounds of formula (III) with an alkylating agent R⁴—X¹, where X¹ is a leaving group such as a chloro, bromo, iodo, methanesulfonyloxy or trifluoromethanesulfonyl oxy group, in the presence of a base such as an alkali metal carbonate (such as potassium carbonate or caesium carbonate) or an amine base (such as dicyclohexylamine), in a solvent such as tetrahydrofuran or dimethylformamide at a temperature between room temperature and the reflux temperature of the solvent for 1-24 hours. The removal of the protecting group P² is as described for reaction scheme 1.

Compounds of formula (Ic), i.e. compounds of formula (I) wherein R⁴ is hydrogen and R⁵ is a biolabile group, may be prepared from compounds of formula (IV) according to reaction scheme 3.

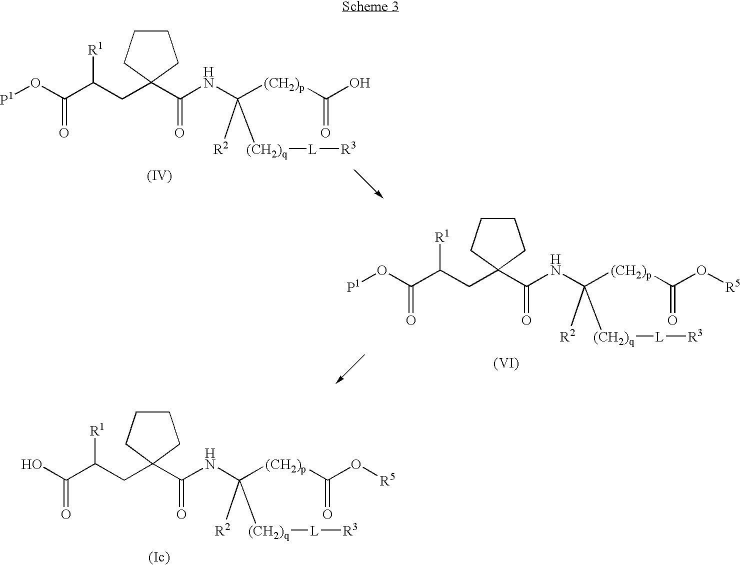

Compounds of formula (VI) may be prepared by methods analogous to those described for reaction scheme 2 by reacting compounds of formula (IV) with R⁵—OH or R⁵—X¹. Deprotection to give compounds of formula (Ic) is as described for reaction scheme 1.

It will be apparent that some embodiments of P¹ and P² are biolabile groups as required for R⁴ and R⁵. Accordingly, some compounds of formula (III) are also compounds of formula (Ic) and some compounds of formula (IV) are also compounds of formula (Ib). Therefore, in those compounds of formula (I) wherein R⁴ or R⁵ can also serve as a carboxyl protecting group, it may be possible to reduce the number of manipulations necessary to arrive at the desired compound.

Compounds of formula (II) may be prepared by coupling an acid of formula (VII) and an amine of formula (VIII) according to reaction scheme 4.

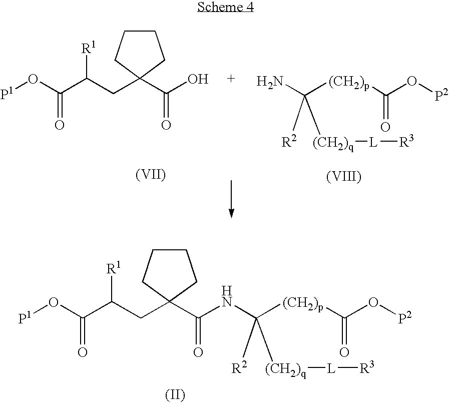

Typical reaction conditions comprise generating the acid chloride of compounds of formula (VII) followed by addition of compounds of formula (VIII), optionally in the presence of an excess of tertiary amine (such as triethylamine, Hünig's base or N-methylmorpholine) in a suitable solvent (such as dichloromethane or tetrahydrofuran) at room temperature for between 1 to 24 hours.

Alternative reaction conditions comprise reacting compounds of formula (VII), WSCDI/1,3-dicyclohexylcarbodiimide (DCC), 1-hydroxybenzotriazole hydrate (HOBT)/HOAT and compounds of formula (VIII), with an excess of a tertiary amine (such as N-methylmorpholine, triethylamine or Hünig's base) in a suitable solvent (for example tetrahydrofuran, dichloromethane or ethyl acetate) at room temperature for between 4 to 48 hours.

Further reaction conditions comprise reacting a compound of formula (VII), PYBOP®/PyBrOP®/Mukaiyama's reagent or TBTU and an excess of compound of formula (VIII), with an excess of tertiary amine (N-methylmorpholine, triethylamine or Hünig's base) in a suitable solvent (such as tetrahydrofuran, dichloromethane or ethyl acetate) at room temperature for 4 to 24 hours.

Preferred reaction conditions comprise reacting compounds of formula (VIII) (1 equiv), a compound of formula (VII) (1-1.1 equiv), hydroxybenzotriazole hydrate (HOBT) (1-1.1 eq) and either WSCDI (1-1.1 eq) and 1-N-methylmorpholine (2 equiv) or TBTU (1-1.1 eq) and Hünig's base (1-2 eq) in dichloromethane at room temperature for about 18 hours.

Compounds of formula (VII) may be prepared by published methods, or by simple modifications of these methods. See for example: A. S. Cook et al., European Patent Application EP 1 258 474 A2 (Pfizer Ltd. et al.); S. Challenger et al., International Patent Application WO02/79143 (Pfizer Ltd. et al.); C. G. Barber et al., International Patent Application WO02/02513 (Pfizer Ltd. et al.); C. J. Cobley et al., Tetrahedron Letts. 42(42), 7481-7483 (2001); S. Challenger, European Patent Application 0 644 176 A1 (Pfizer Ltd.).

Compounds of formula (VIII) may be prepared by a variety of routes depending on the nature of the aromatic heterocyclic group L, as further illustrated hereinafter.

Compounds of formula (VIIIa) wherein Y¹ is CH or N, i.e. compounds of formula (VIII) where L is oxazole or [1.3.4]oxadiazole, may be prepared from an acid of formula (IX), wherein P³ is an amine protecting group such as t-butyloxycarbonyl (BOC), benzyloxycarbonyl (Cbz) or 9-fluorenylmethyloxycarbonyl (Fmoc), and an aminoketone or acylhydrazide of formula (X) according to reaction scheme 5.

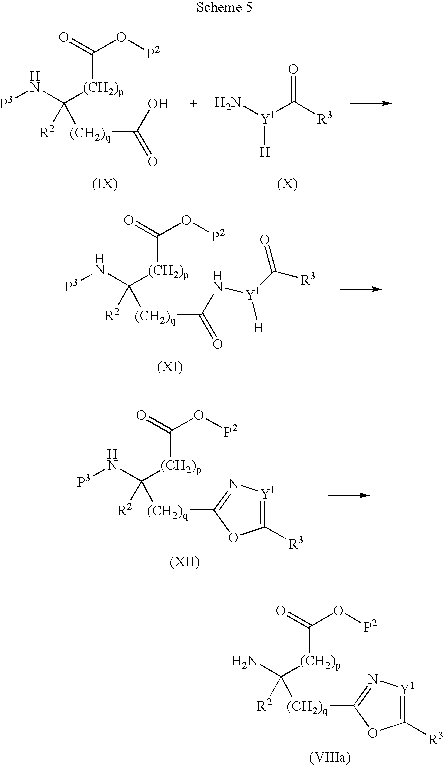

The condensation of acid of formula (IX) and amine or hydrazide of formula (X) may be achieved by the methods set out for the formation of an amide bond in reaction scheme 4 above. Preferred methods are either conversion of the acid to the corresponding acid chloride with thionyl chloride in dimethylformamide and dichloromethane at room temperature for 18 hours followed by addition of triethylamine and the amine or hydrazide at 0° C. and stirring for 4 to 6 hours, or treating a solution of the two components in dichloromethane with WSCDI, NMM and 1-hydroxybenzotriazole at room temperature for 18 hours.

The cyclization of the compounds of formula (XI) to give the aromatic heterocyclic compounds of formula (XII) may be carried out according to a variety of standard methods. See for example “Comprehensive Heterocyclic Chemistry II”, Pergammon, New York, 1996.

When Y¹ is CH preferred methods include treating the compounds of formula (XI) with phosphorus oxychloride (2-4 eq) in toluene at about 100° C. for about 1-2 hours, treating the compounds of formula (XI) with triphenylphosphine (2 eq), triethylamine (4 eq) and iodine in tetrahydrofuran at −78° C. for 1 hour, −20° C. for 1 hour, then at room temperature for 1 hour, and treating the compounds of formula (XI) with 2,6-di-tert-butylpyridine (1.2 eq), 1,2-dibromotetrachloroethane (1.2 eq) and triphenylphosphine (1.2 eq) in dichloromethane at room temperature for 30 minutes, followed by 1,8-diazabicyclo[5.4.0]undec-7-ene (1.2 eq) at room temperature for 1 hour.

When Y¹ is N a preferred method is to treat the compounds of formula (XI) with 2-chloro-1,3-dimethylimidazolinium chloride (1.2 eq) and triethylamine (2 eq) in dichloromethane at room temperature for 18 hours.

Finally, the protecting group P³ may be removed by the appropriate standard method, as set out in “Protecting Groups in Organic Synthesis” by T. W. Greene and P. Wutz. When P³ is Cbz, suitable conditions include hydrogenolysis with hydrogen gas or a hydrogen donor such as ammonium formate in a suitable polar solvent such as tetrahydrofuran, methanol or ethanol, in the presence of a catalyst such as palladium-on-carbon or palladium hydroxide, and hydrolysis with a strong acid such as HBr, acetic acid, methanesulfonic acid or trifluoromethanesulfonic acid. Preferred conditions include treatment with hydrogen gas at 100-350 kPa in tetrahydrofuran or ethanol in the presence of 10% Pd/C (0.1 g catalyst/1 g of compound) at room temperature for 15-50 hours and treatment with HBr (15 eq) in acetic acid at room temperature for 1-18 hours. When P³ is BOC, suitable conditions include treatment with excess of a strong acid such as hydrogen chloride or trifluoroacetic acid in a suitable solvent such as dichloromethane, ethyl acetate or dioxan. Preferred conditions include treatment with trifluoroacetic acid in dichloromethane at room temperature for 1-2 hours and treatment with an excess of 4M hydrogen chloride in dioxan at room temperature for 18 hours.

Compounds of formula (VIIIb), i.e. compounds of formula (VIII) where L is [1.2.4]oxadiazole, may be prepared from an acid of formula (IX), wherein P³ is an amine protecting group as previously described, and an amidoxime of formula (XIII) according to reaction scheme 6.

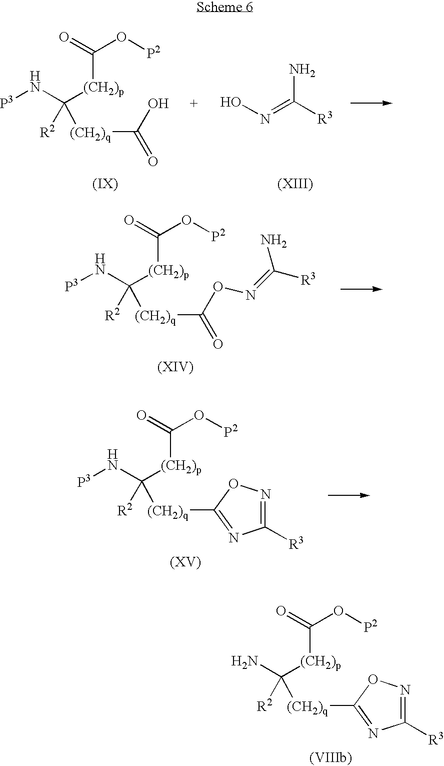

The condensation of acid of formula (IX) and amidoxime of formula (XIII) may be achieved by the methods analogous to those set out for the formation of an amide bond in reaction scheme 4 above.

Cyclization to give compounds of formula (XV) may be achieved by a variety of standard methods. See for example “Comprehensive Heterocyclic Chemistry II”, Pergammon, New York, 1996. A preferred method is to heat the compounds to about 110° C. to 120° C.

Finally, the protecting group P³ may be removed by the appropriate standard method, as set out above for reaction scheme 5.

Compounds of formula (VIIIc), i.e. compounds of formula (VIII) where L is oxazole, may be prepared from an acid of formula (IX), wherein P³ is an amine protecting group as previously described, and an aminoalcohol of formula (XVI) according to reaction scheme 7.

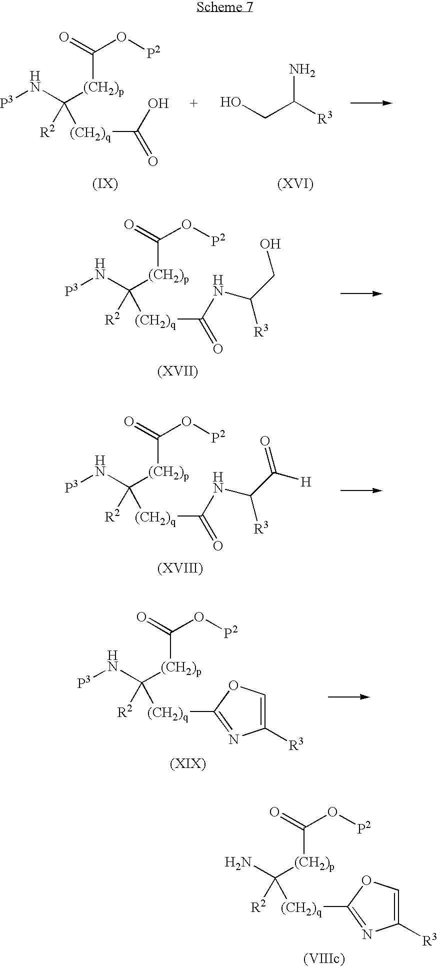

The condensation of acid of formula (IX) and aminoalcohol of formula (XVI) may be achieved by the methods set out for the formation of an amide bond in reaction scheme 4 above.

Compounds of formula (XVII) may be oxidised to provide aldehydes of formula (XVIII) achieved by a variety of standard methods. A preferred method is to treat a solution of the compound in dichloromethane with Dess-Martin periodinane at room temperature for 2 to 3 hours.

Cyclization to give compounds of formula (XIX) may be achieved by a variety of standard methods. See for example “Comprehensive Heterocyclic Chemistry II”, Pergammon, New York, 1996.

Finally, the protecting group P³ may be removed by the appropriate standard method, as set out above for reaction scheme 5.

Compounds of formula (VIIId), i.e. compounds of formula (VIII) where L is pyrazole, triazole, tetrazole or imidazole (depending on whether Y², Y³ and Y⁴ are nitrogen or carbon) p is 0 and q, may be prepared from a lactone of formula (XX), wherein P³ is an amine protecting group as previously described, and a pyrazole or imidazole of formula (XXI) according to reaction scheme 8.

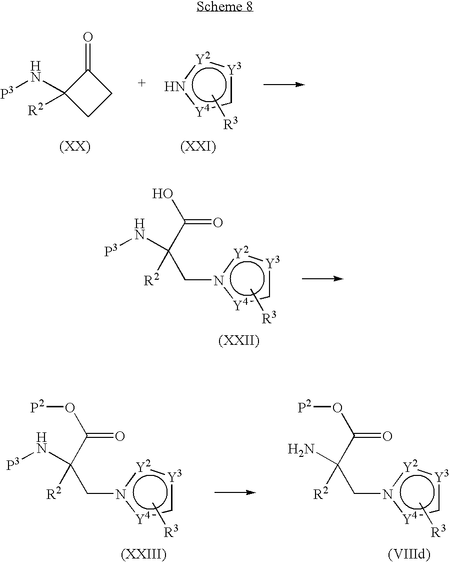

The formation of acid of formula (XXII) may be achieved by reacting an imidazole or pyrazole, triazole, tetrazole or imidazole of formula (XXI) and a lactone of formula (XX), optionally in the presence of a base such as potassium carbonate or 1,8-diazabicyclo[5.4.0]undec-7-ene, in a suitable solvent such as acetonitrile or dimethylformamide at a temperature between 0° C. and the reflux temperature of the solvent for 1 hour to several days. Preferably an equimolar mixture of the two components in acetonitrile is stirred at room temperature for 5 days. The reaction is particularly suitable for the preparation of imidazoles (i.e. when Y² and Y⁴ are carbon and Y³ is nitrogen).

Conversion of acid of formula (XXII) to ester of formula (XXIII) can be achieved using the methods described for the preparation of compounds of formula (V) in reaction scheme 2 above.

Finally, the protecting group P³ may be removed by the appropriate standard method, as set out above for reaction scheme 5.

Alternatively, compounds of formula (XXIII) of reaction scheme 8 may be prepared from an aziridine of formula (XXIV) and a pyrazole, triazole, tetrazole or imidazole of formula (XXI) or (XXV), wherein X² is a leaving group such as a halo or sulfonyloxy group, preferably an iodo group, according to reaction scheme 9.

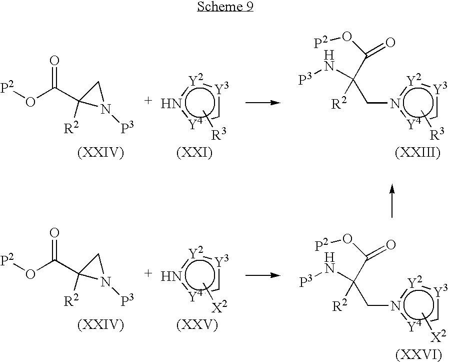

The formation of ester of formula (XXIII) may be achieved by reacting a pyrazole, triazole, tetrazole or imidazole of formula (XXI) and an aziridine of formula (XXIV) in the presence of a Lewis acid in a suitable solvent at a temperature between 0° C. and the reflux temperature of the solvent for 1-24 hours. Preferably an equimolar mixture of the two components in dichloromethane is treated with boron trifluoride etherate at room temperature for 6 hours. The reaction is particularly suitable for the preparation of pyrazoles (i.e. when Y² is nitrogen and Y³ and Y⁴ are carbon).

Esters of formula (XXVI) may be prepared in an analogous manner.

Conversion of acid of formula (XXVI) to ester of formula (XXIII) can be achieved by reaction with a trialkylstannane R³—Sn(alkyl)₃ using the method known as the Stille coupling (Organic Reactions 50, 1, 1997). Preferably, a solution of compound of formula (XXVI) and tris(dibenzylideneacetone)dipalladium(0) (0.05 eq) in dimethylformamide is treated with R³—Sn(butyl)₃ (1.2 eq) and CuI (0.2 eq) and heated at 60° C. for 90 minutes.

Compounds of formula (VIIIe), i.e. compounds of formula (VIII) where p is 0 and R2 is hydrogen, may be prepared from a compound of formula (XXVII), wherein X³ is a leaving group such as a halo or sulfonyloxy group, preferably a bromo group, and a dihydropyrazine of formula (XXVIII) according to reaction scheme 10.

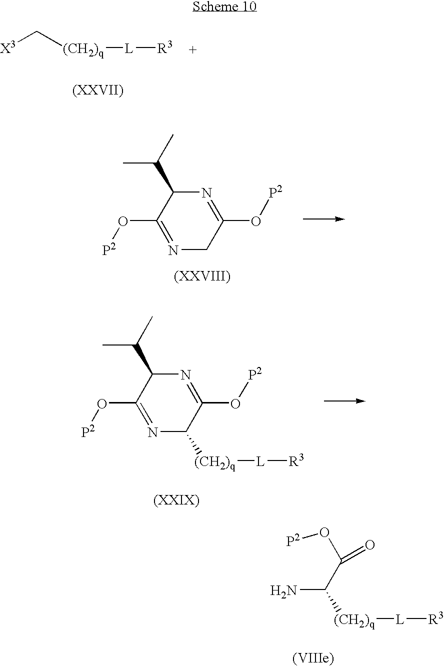

The formation of compound of formula (XXIX) may be achieved by reacting an alkylating agent of formula (XXVII) and a dihydropyrazine of formula (XXVIII), also known as a Schöllkopf auxiliary) according to the methods described in Angew. Chem. Int. Ed. Engl. 20, 798, 1981. Generally, the dihydropyrazine is treated with a strong base, such as an alkali metal alkyl or amide, at a temperature below −50° C., in an ether solvent such as diethyl ether, dioxan or tetrahydrofuran, followed by addition of the alkylating agent. Preferably a solution of dihydropyrazine in tetrahydrofuran at −60° C. is treated with butyllithium (1.2 eq) and then the alkylating agent, and the mixture is stirred at −60° C. for 18 hours.

The resulting dihydropyrazine of formula (XXIX) may be hydrolysed in the presence of acid to give the compound of formula (VIIIe). Preferred conditions include treatment of a solution of the dihydropyrazine with 0.25M HCl in tetrahydrofuran (2-3 eq) at room temperature for 5-18 hours.

A pharmaceutically acceptable salt of a compound of the formula (I) may be readily prepared by mixing together solutions of a compound of the formula (I) and the desired acid or base, as appropriate. The salt may precipitate from solution and be collected by filtration or may be recovered by evaporation of the solvent.

Procedures for isolating the desired products will be well-known to those skilled in the art with reference to literature precedents and the Examples and Preparations hereto.

The compounds of the invention can be administered alone but will generally be administered in admixture with a suitable pharmaceutical excipient, diluent or carrier selected with regard to the intended route of administration and standard pharmaceutical practice.

For example, the compounds of the invention can be administered orally, buccally or sublingually in the form of tablets, capsules, multi-particulates, gels, films, ovules, elixirs, solutions or suspensions, which may contain flavouring or colouring agents, for immediate-, delayed-, modified-, sustained-, pulsed- or controlled-release applications. The compounds of the invention may also be administered as fast-dispersing or fast-dissolving dosage forms or in the form of a high energy dispersion or as coated particles. Suitable formulations of the compounds of the invention may be in coated or uncoated form, as desired.

Such solid pharmaceutical compositions, for example, tablets, may contain excipients such as microcrystalline cellulose, lactose, sodium citrate, calcium carbonate, dibasic calcium phosphate, glycine and starch (preferably corn, potato or tapioca starch), disintegrants such as sodium starch glycollate, croscarmellose sodium and certain complex silicates, and granulation binders such as polyvinylpyrrolidone, hydroxypropylmethylcellulose (HPMC), hydroxypropylcellulose (HPC), sucrose, gelatin and acacia. Additionally, lubricating agents such as magnesium stearate, stearic acid, glyceryl behenate and talc may be included.

### GENERAL EXAMPLE

A formulation of the tablet could typically contain between about 0.01 mg and 500 mg of active compound whilst tablet fill weights may range from 50 mg to 1000 mg. An example of a formulation for a 10 mg tablet is illustrated below:

| Ingredient | % w/w |
|---|---|
| Free acid, Free base or Salt form | 10.000* |
| Lactose | 64.125 |
| Starch | 21.375 |
| Croscarmellose Sodium | 3.000 |
| Magnesium Stearate | 1.500 |

*Quantity adjusted in accordance with drug activity.

The tablets are manufactured by a standard process, for example, direct compression or a wet or dry granulation process. The tablet cores may be coated with appropriate overcoats.

Solid compositions of a similar type may also be employed as fillers in gelatin or HPMC capsules. Preferred excipients in this regard include lactose, starch, a cellulose, milk sugar or high molecular weight polyethylene glycols. For aqueous suspensions and/or elixirs, the compounds of the invention may be combined with various sweetening or flavouring agents, colouring matter or dyes, with emulsifying and/or suspending agents and with diluents such as water, ethanol, propylene glycol and glycerin, and combinations thereof.

Modified release and pulsatile release dosage forms may contain excipients such as those detailed for immediate release dosage forms together with additional excipients that act as release rate modifiers, these being coated on and/or included in the body of the device. Release rate modifiers include, but are not exclusively limited to, hydroxypropylmethyl cellulose, methyl cellulose, sodium carboxymethylcellulose, ethyl cellulose, cellulose acetate, polyethylene oxide, Xanthan gum, Carbomer, ammonio methacrylate copolymer, hydrogenated castor oil, carnauba wax, paraffin wax, cellulose acetate phthalate, hydroxypropylmethyl cellulose phthalate, methacrylic acid copolymer and mixtures thereof. Modified release and pulsatile release dosage forms may contain one or a combination of release rate modifying excipients. Release rate modifying excipients may be present both within the dosage form i.e. within the matrix, and/or on the dosage form, i.e. upon the surface or coating.

Fast dispersing or dissolving dosage formulations (FDDFs) may contain the following ingredients: aspartame, acesulfame potassium, citric acid, croscarmellose sodium, crospovidone, diascorbic acid, ethyl acrylate, ethyl cellulose, gelatin, hydroxypropylmethyl cellulose, magnesium stearate, mannitol, methyl methacrylate, mint flavouring, polyethylene glycol, fumed silica, silicon dioxide, sodium starch glycolate, sodium stearyl fumarate, sorbitol, xylitol. The terms dispersing or dissolving as used herein to describe FDDFs are dependent upon the solubility of the drug substance used i.e. where the drug substance is insoluble a fast dispersing dosage form can be prepared and where the drug substance is soluble a fast dissolving dosage form can be prepared.

The compounds of the invention can also be administered parenterally, for example, intracavernouslly, intravenously, intra-arterially, intraperitoneally, intrathecally, intraventricularly, intraurethrally, intrasternally, intracranially, intramuscularly or subcutaneously, or they may be administered by infusion or needleless injection techniques. For such parenteral administration they are best used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well-known to those skilled in the art.

The following dosage levels and other dosage levels herein are for the average human subject having a weight range of about 65 to 70 kg. The skilled person will readily be able to determine the dosage levels required for a subject whose weight falls outside this range, such as children and the elderly.

For oral and parenteral administration to human patients, the daily dosage level of the compounds of the invention will usually be from 0.01 mg/kg to 10 mg/kg (in single or divided doses).

Thus tablets or capsules of the compound of the invention may contain from 1 mg to 500 mg of active compound for administration singly or two or more at a time, as appropriate. The physician in any event will determine the actual dosage which will be most suitable for any individual patient and it will vary with the age, weight and response of the particular patient. The above dosages are exemplary of the average case. There can, of course, be individual instances where higher or lower dosage ranges are merited and such are within the scope of this invention.

The compounds of the invention can also be administered intranasally or by inhalation and are conveniently delivered in the form of a dry powder inhaler or an aerosol spray presentation from a pressurised container, pump, spray, atomiser or nebuliser, with or without the use of a suitable propellant, e.g. dichlorodifluoromethane, trichlorofluoromethane, dichlorotetrafluoroethane, a hydrofluoroalkane such as 1,1,1,2-tetrafluoroethane (HFA 134A [trade mark]) or 1,1,1,2,3,3,3-heptafluoropropane (HFA 227EA [trade mark]), carbon dioxide or other suitable gas. In the case of a pressurised aerosol, the dosage unit may be determined by providing a valve to deliver a metered amount. The pressurised container, pump, spray, atomiser or nebuliser may contain a solution or suspension of the active compound, e.g. using a mixture of ethanol and the propellant as the solvent, which may additionally contain a lubricant, e.g. sorbitan trioleate. Capsules and cartridges (made, for example, from gelatin) for use in an inhaler or insufflator may be formulated to contain a powder mix of a compound of the invention and a suitable powder base such as lactose or starch.

Aerosol or dry powder formulations are preferably arranged so that each metered dose or “puff” contains from 1 to 50 mg of a compound of the invention for delivery to the patient. The overall daily dose with an aerosol will be in the range of from 1 to 50 mg which may be administered in a single dose or, more usually, in divided doses throughout the day.

Alternatively, the compounds of the invention can be administered in the form of a suppository or pessary, or they may be applied topically in the form of a gel, hydrogel, lotion, solution, cream, ointment or dusting powder. The compounds of the invention may also be dermally or transdermally administered, for example, by the use of a skin patch. They may also be administered by the pulmonary, vaginal or rectal routes.

They may also be administered by the ocular route, particularly for treating disorders of the eye. For ophthalmic use, the compounds can be formulated as micronised suspensions in isotonic, pH adjusted, sterile saline, or, preferably, as solutions in isotonic, pH adjusted, sterile saline, optionally in combination with a preservative such as a benzylalkonium chloride. Alternatively, they may be formulated in an ointment such as petrolatum.

For application topically to the skin, the compounds of the invention can be formulated as a suitable ointment containing the active compound suspended or dissolved in, for example, a mixture with one or more of the following: mineral oil, liquid petrolatum, white petrolatum, propylene glycol, polyoxyethylene polyoxypropylene compound, emulsifying wax and water. Alternatively, they can be formulated as a suitable lotion or cream, suspended or dissolved in, for example, a mixture of one or more of the following: mineral oil, sorbitan monostearate, a polyethylene glycol, liquid paraffin, polysorbate 60, cetyl esters wax, cetearyl alcohol, 2-octyldodecanol, benzyl alcohol and water.

The compounds of the invention may also be used in combination with a cyclodextrin. Cyclodextrins are known to form inclusion and non-inclusion complexes with drug molecules. Formation of a drug-cyclodextrin complex may modify the solubility, dissolution rate, bioavailability and/or stability property of a drug molecule. Drug-cyclodextrin complexes are generally useful for most dosage forms and administration routes. As an alternative to direct complexation with the drug the cyclodextrin may be used as an auxiliary additive, e.g. as a carrier, diluent or solubiliser. Alpha-, beta- and gamma-cyclodextrins are most commonly used and suitable examples are described in WO-A-91/11172, WO-A-94/02518 and WO-A-98/55148.

For treating cardiovascular disorders, particular hypertension, the compounds of the invention may be combined with one or more active ingredient selected from the list:

a) angiotensin receptor blockers (ARB), such as losartan, valsartan, telmisartan, candesartan, irbesartan, eprosartan and olmesartan;

b) calcium channel blockers (CCB) such as amlodipine;

c) statins, such as atorvastatin;

d) PDE5 inhibitors, such as sildenafil, tadalafil, vardenafil, 5-[2-ethoxy-5-(4-ethylpiperazin-1-ylsulphonyl)pyridin-3-yl]-3-ethyl-2-[2-methoxyethyl]-2,6-dihydro-7H-pyrazolo[4,3-d]pyrimidin-7-one; 5-(5-acetyl-2-butoxy-3-pyridinyl)-3-ethyl-2-(1-ethyl-3-azetidinyl)-2,6-dihydro-7H-pyrazolo[4,3-d]pyrimidin-7-one and; the pyrazolo[4,3-d]pyrimidin-4-ones disclosed in WO00/27848 particularly N-[[3-(4,7-dihydro-1-methyl-7-oxo-3-propyl-1H-pyrazolo[4,3-d]-pyrimidin-5-yl)-4-propxyphenyl]sulfonyl]-1-methyl2-pyrrolidinepropanamide [DA-8159 (Example 68 of WO00/27848)];

e) beta blockers, such as atenolol or carvedilol;

f) ACE inhibitors, such as quinapril, enalapril and lisinopril;

g) alpha-blockers such as doxazosin;

h) selective aldosterone receptor antagonists (SARA), such as eplerenone or spironolactone;

i) imidazoline I₁ agonists, such as rilmenidine and moxonidine; and

j) endothelin receptor antagonists and endothelin converting enzyme inhibitors.

For treating FSAD, the compounds of the invention may be combined with one or more active ingredient selected from the list:

a) PDE5 inhibitors, such as sildenafil, tadalafil, vardenafil, 5-[2-ethoxy-5-(4-ethylpiperazin-1-ylsulphonyl)pyridin-3-yl]-3-ethyl-2-[2-methoxyethyl]-2,6-dihydro-7H-pyrazolo[4,3-d]pyrimidin-7-one; 5-(5-acetyl-2-butoxy-3-pyridinyl)-3-ethyl-2-(1-ethyl-3-azetidinyl)-2,6-dihydro-7H-pyrazolo[4,3-d]pyrimidin-7-one and; the pyrazolo[4,3-d]pyrimidin-4-ones disclosed in WO00/27848 particularly N-[[3-(4,7-dihydro-1-methyl-7-oxo-3-propyl-1H-pyrazolo[4,3-d]-pyrimidin-5-yl)-4-propxyphenyl]sulfonyl]-1-methyl2-pyrrolidinepropanamide [DA-8159 (Example 68 of WO-00/27848)];

b) dopaminergic agents, preferably apomorphine or a selective D₂, D₃ or D₂/D₃ agonist such as, pramipexole and ropirinol (as claimed in WO-0023056), PNU95666 (as claimed in WO-00/40226);

c) melanocortin receptor agonists, such as melanotan II; PT-14; PT-141;

compounds claimed in WO-99/64002, WO-00/74679, WO-99/55679, WO-01/05401, WO-00/58361, WO-01/14879, WO-01/13112 and WO-99/54358; selective MC4 receptor agonists such as those disclosed by Martin et al [European Journal of Pharmacology, 454 71-79 (2002)] particularly (N-[(3R)-1,2,3,4-tetrahydroisoquinolinium-3-ylcarbonyl]-(1R)-1-(4-chlorobenzyl)-2-[4-cyclohexyl-4-(1H-1,2,4-triazol-1-ylmethyl)piperidin-1-yl]-2-oxoethylamine (THIQ); and selective MC3 receptor agonists

d) selective estrogen receptor modulators (SERMs) such as lasofoxifene and raloxifene;

e) tibolone;

f) an androgen such as androsterone, dehydro-androsterone, testosterone, androstanedione and a synthetic androgen; and

g) an oestrogen, such as oestradiol, oestrone, oestriol and a synthetic estrogen, such as oestrogen benzoate.

For treating MED, the compounds of the invention may be combined with one or more active ingredient selected from the list:

a) PDE5 inhibitors, such as sildenafil, tadalafil, vardenafil, 5-[2-ethoxy-5-(4-ethylpiperazin-1-ylsulphonyl)pyridin-3-yl]-3-ethyl-2-[2-methoxyethyl]-2,6-dihydro-7H-pyrazolo[4,3-d]pyrimidin-7-one; 5-(5-acetyl-2-butoxy-3-pyridinyl)-3-ethyl-2-(1-ethyl-3-azetidinyl)-2,6-dihydro-7H-pyrazolo[4,3-d]pyrimidin-7-one and; the pyrazolo[4,3-d]pyrimidin-4-ones disclosed in WO00/27848 particularly N-[[3-(4,7-dihydro-1-methyl-7-oxo-3-propyl-1H-pyrazolo[4,3-d]-pyrimidin-5-yl)-4-propxyphenyl]sulfonyl]-1-methyl2-pyrrolidinepropanamide [DA-8159 (Example 68 of WO00/27848)];

b) dopaminergic agents, preferably apomorphine or a selective D₂, D₃ or D₂/D₃ agonist such as, pramipexole and ropirinol (as claimed in WO-00/23056), PNU95666 (as claimed in WO-00/40226); and

c) melanocortin receptor agonists, such as melanotan II; PT-14; PT-141; compounds claimed in WO-99/64002, WO-00/74679, WO-99/55679, WO-01/05401, WO-00/58361, WO-01/14879, WO-01/13112 and WO-99/54358; selective MC4 receptor agonists such as those disclosed by Martin et al [European Journal of Pharmacology, 454 71-79 (2002)] particularly (N-[(3R)-1,2,3,4-tetrahydroisoquinolinium-3-ylcarbonyl]-(1R)-1-(4-chlorobenzyl)-2-[4-cyclohexyl-4-(1H-1,2,4-triazol-1-ylmethyl)piperidin-1-yl]-2-oxoethylamine (THIQ); and selective MC3 receptor agonists.

If a combination of active agents are administered, then they may be administered simultaneously, separately or sequentially.

The invention also includes the following aspects. The preferred embodiments specified hereinabove for the first aspect extend to these aspects.

The invention additionally includes:

(i) A pharmaceutical composition including a compound of the invention, together with a pharmaceutically acceptable excipient, diluent or carrier.

(ii) A compound of the invention for use as a medicament.

(iii) The use of a compound of the invention as a medicament for treating or preventing a condition for which a beneficial therapeutic response can be obtained by the inhibition of neutral endopeptidase.

(iv) The use of a compound of the invention as a medicament for treating or preventing cardiovascular diseases and conditions, preferably essential hypertension, pulmonary hypertension, secondary hypertension, isolated systolic hypertension, hypertension associated with diabetes, hypertension associated with atherosclerosis, renovascular hypertension, congestive heart failure, angina, stroke, glaucoma, impaired renal function, renal failure, obesity, metabolic diseases (including Metabolic Syndrome), diabetes and impaired glucose tolerance, including complications thereof, such as diabetic retinopathy and diabetic neuropathy.

(v) A method of treating or preventing cardiovascular diseases and conditions (preferably essential hypertension, pulmonary hypertension, secondary hypertension, isolated systolic hypertension, hypertension associated with diabetes, hypertension associated with atherosclerosis, renovascular hypertension, congestive heart failure, angina, stroke, glaucoma, impaired renal function, renal failure, obesity, metabolic diseases (including Metabolic Syndrome), diabetes and impaired glucose tolerance, including complications thereof, such as diabetic retinopathy and diabetic neuropathy) in a mammal including treating said mammal with an effective amount of a compound of the invention.

(vi) A cardiovascular disease treating pharmaceutical composition comprising a compound of the invention together with a pharmaceutically acceptable excipient, diluent or carrier.

(vii) A compound of the invention for use in treating or preventing cardiovascular diseases and conditions.

(viii) The use of a compound of the invention in the manufacture of a medicament for treating or preventing cardiovascular disease and conditions.

The invention is illustrated by the following non-limiting examples in which the following abbreviations and definitions are used:

| | |
|---|---|
| DCC/DCCI | dicyclohexylcarbodiimide |
| ES⁺ | electrospray ionisation positive scan |
| ES⁻ | electrospray ionisation negative scan |
| m/z | mass spectrum peak |
| MS | mass spectrum |
| Prep | preparation |
| TS⁺ | thermospray ionisation positive scan |
| WSCDI | 1-(3-dimethylaminopropyl)-3- |
| | ethylcarbodiimide hydrochloride |

¹H Nuclear magnetic resonance (NMR) spectra were in all cases consistent with the proposed structures. Characteristic chemical shifts (6) are given in parts-per-million downfield from tetramethylsilane using conventional abbreviations for designation of major peaks: e.g. s, singlet; d, doublet; t, triplet; q, quartet; m, multiplet; br, broad. The following abbreviations have been used for common solvents: CDCl₃, deuterochloroform; DMSO, dimethylsulphoxide. The abbreviation psi means pounds per square inch and LRMS means low resolution mass spectrometry. Where thin layer chromatography (TLC) has been used it refers to silica gel TLC using silica gel 60 F₂₅₄ plates, R_{f} is the distance travelled by a compound divided by the distance travelled by the solvent front on a TLC plate. Melting points were determined using a Perkin Elmer DSC7 at a heating rate of 20° C./minute).

### EXAMPLE 1

(2S)-2-{1-[(1S)-1-Ethoxycarbonyl-2-(4-methyl-5-phenyl-oxazol-2-yl)-ethylcarbamoyl]-cyclopentylmethyl}-4-methoxy-butyric Acid

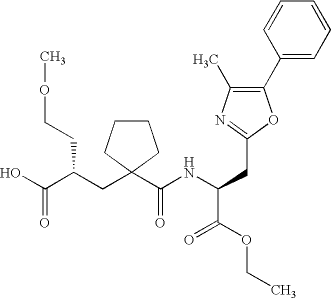

Trifluoroacetic acid (4 mL) was added to a solution of the di-ester of preparation 76 (1.2 g, 2 mmol) in dichloromethane (8 mL) and the reaction mixture stirred at room temperature for 2 hours. The reaction mixture was concentrated in vacuo and azeotroped with toluene (2×10 mL). The crude product was purified by column chromatography on silica gel, eluting with dichloromethane:methanol (99:1 to 95:5) to yield the title product, 1.04 g.

[α]=−18.94 (1.320 mg/mL) in methanol; ¹HNMR (CDCl₃, 400 MHz): 1.22 (m, 3H), 1.64 (brm, 12H), 2.40 (s, 3H), 2.52 (m, 1H), 3.26 (s, 3H), 3.41 (m, 2H), 3.50 (m, 2H), 4.20 (m, 2H), 4.96 (m, 1H), 7.35 (m, 1H), 7.44 (m, 2H), 7.57 (m, 2H); MS ES+ m/z 501 [MH]⁺.

The following compounds of the general formula shown below were made by the method described in example 1 using the appropriate tert-butyl ester starting material.

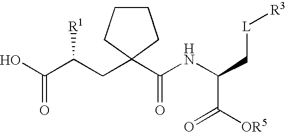

| Ex | R¹ | L-R³ | R⁵ | Data |
|---|---|---|---|---|
| 2 | methoxy- ethyl | 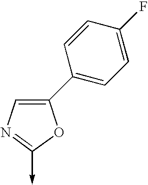 | ethyl | [α] = −16.67(0.600 mg/mL) in methanol; ¹HNMR (CDCl₃, 400MHz): 1.22(m, 3H), 1.62(brm, 12H), 2.55(m, 1H), 3.23(s, 3H), 3.30-3.45(brm, 4H), 4.21(m, 2H), 4.98(m, 1H), 6.92(d, 1H), 7.12(t, 2H), 7.20(s, 1H), 7.58(m, 2H); MS ES− m/z 503[M − H]⁻ |
| 3 | methoxy- ethyl | 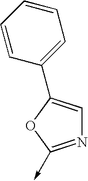 | ethyl | [α] = −16.14(0.85 mg/mL) in methanol; ¹HNMR (CDCl₃, 400MHz): 1.26(t, 3H), 1.52-2.20(brm 12H), 2.58(s, 1H), 3.28(s, 3H), 3.37-3.59(m, 4H), 4.21(d, 2H), 4.99(s, 1H), 6.85(s, 1H), 7.34-7.47(m, 4H), 7.60(m, 2H); MS ES− m/z 485 [M − H]⁻ |
| 4 | propyl | 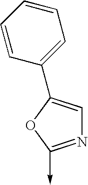 | ethyl | [α] = −19.50(0.40 mg/mL) in methanol; ¹HNMR (CDCl₃, 400MHz): 0.83(t, 3H), 1.24(m, 6H), 1.63(m, 8H), 2.01(m, 2H), 2.19(m, 1H), 2.38(m, 1H), 3.47(m, 2H), 4.24(m, 2H), 5.03(d, 1H), 6.94(s, 1H), 7.17-7.48(m, 6H); MS ES+ m/z 471 [MH]⁻ |
| 5 | methoxy- ethyl | 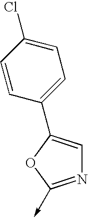 | ethyl | [α] = −21.4(0.7 mg/mL) in methanol ¹HNMR (CDCl₃, 400MHz): 1.25(t, 3H), 1.50-2.20(brm, 12H), 2.57(m, 1H), 3.26(s, 3H), 3.40(m, 2H), 3.47(d, 2H), 4.21(m, 2H), 4.99(m, 1H), 6.85(d, 1H), 7.24(s, 1H), 7.40(d, 2H), 7.54(d, 2H) MS APCl m/z 521 [MH]⁻ |
| 6 | propyl | 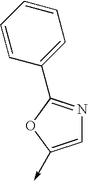 | ethyl | [α] = −43.87(2.02 mg/mL) in methanol; ¹HNMR (CDCl₃, 400MHz): 0.82(t, 3H), 1.22(m, 6H), 1.71(m, 8H), 2.17(m, 3H), 2.38(m, 1H), 3.19(dd, 1H), 3.24(dd, 1H), 4.19(m, 2H), 4.88(m, 1H), 7.36(d, 1H), 7.49(m, 3H), 7.57(s, 1H), 8.05(m, 2H); MS ES+ m/z 493 [MNa]⁺ |
| 7 | methoxy- ethyl | 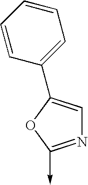 | isobutyl | ¹HNMR (CDCl₃, 400MHz): 0.88(d, 6H), 1.64(m, 6H), 2.01(m, 7H), 3.28(s, 3H), 3.37(m, 2H), 3.46(m, 2H), 3.95(m, 2H), 5.07(m, 1H), 7.03(m, 1H), 7.30(m, 1H), 7.36(m, 1H), 7.41(m, 2H), 7.59(m, 2H), 10.06(brs, 1H) MS ES− m/z 513 [MH]⁻ |
| 8 | methoxy- ethyl | 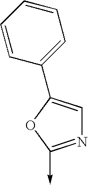 | isopropyl | ¹HNMR (CDCl₃, 400MHz): 1.20(dd, 3H), 1.23(dd, 3H), 1.63(m, 8H), 1.91(m, 2H), 2.07(m, 2H), 2.14(m, 1H), 3.22(s, 3H), 3.39(m, 2H), 3.43(m, 3H), 5.02(m, 1H), 6.95(d, 1H), 7.28(m, 1H), 7.36(m, 1H), 7.40(m, 2H), 7.59(m, 2H) MS APCl m/z 501 [MH]⁻ |
| 9 | methoxy- ethyl | 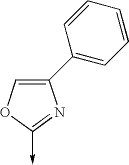 | ethyl | [α] = −11.74(0.92 mg/mL) in methanol ¹HNMR (CDCl₃, 400MHz): 1.26(t, 3H), 1.73(m, 8H), 1.92(m, 1H), 2.05(m, 1H), 2.18(m, 2H), 2.56(m, 1H), 3.22(s, 3H), 3.30-3.47(brm, 4H), 4.18(m, 2H), 4.99(m, 1H), 7.19(d, 1H), 7.37(m, 1H), 7.40(m, 2H), 7.63(m, 2H), |
| # 7.85(m, 1H) MS ES+ m/z 487 [MH]⁺ | | | | |
| 10 | methoxy- ethyl | 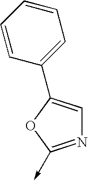 | propyl | [α] = −10.90(2.00 mg/mL) in methanol ¹HNMR (DMSOD₆, 400MHz): 0.91(m, 3H), 1.46-2.20(brm, 14H), 2.57(s, 1H), 3.24(s, 3H), 3.38(d, 2H), 3.49(m, 2H), 4.14(m, 2H), 5.02(s, 1H), 6.98(d, 1H), 7.20-7.51(m, 6H) MS ES+ m/z 501 [MH]⁺ |
| 11 | methoxy- ethyl | 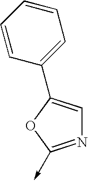 | butyl | [α] = −11.52(3.30 mg/mL) in methanol ¹HNMR (CDCl₃, 400MHz): 0.85(t, 3H), 1.37(m, 2H), 1.51-2.30(brm, 14H), 2.49(s, 1H), 3.23(s, 3H), 3.38(m, 3H), 3.61(m, 2H), 4.19(m, 1H), 5.08(m, 1H), 7.12(d, 1H), 7.20-7.68(m, 6H) MS ESI m/z 515 [MH]⁺ |
| 12 | methoxy- ethyl | 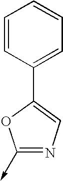 | benzyl | [α] = −17.82(1.10 mg/mL) in methanol ¹HNMR (DMSOD₆, 400MHz): 1.18-1.47(m, 6H), 1.51-1.62(m, 2H), 1.76(m, 1H), 1.84(m, 2H), 1.98(m, 1H), 2.17(m, 1H), 3.08(s, 3H), 3.20-3.32(m, 3H), 3.38(m, 1H), 4.84(m, 1H), 5.07(m, 2H), |
| # 7.31(m, 5H), 7.40(m, 2H), 7.57(m, 1H), 7.63(m, 2H), 8.14(m, 1H) MS ES+ m/z 549 [MH]⁺ | | | | |
| 13 | propyl | 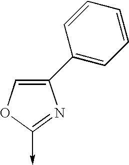 | ethyl | ¹HNMR (CDCl₃, 400MHz): 0.81(m, 3H), 1.16-1.37(m, 6H), 1.45-1.89(m, 7H), 1.91(m, 1H), 2.00(m, 2H), 2.11(m, 1H), 2.36(m, 1H), 3.38(m, 2H), 4.18(m, 2H), 5.02(m, 1H), 7.06(m, 1H), 7.31(m, 1H), 7.39(m, 2H), 7.65(m, 2H), 7.84(m, 1H) MS ES+ m/z 471 [MH]⁺ |
| 14 | propyl | 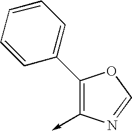 | ethyl | [α] = +18.85 (1.433 mg/mL) in methanol; ¹HNMR (CDCl₃, 400MHz): 0.86(t, 3H), 1.06(t, 3H), 1.18-1.39(m, 4H), 1.47-1.77(m, 7H), 1.82(m, 1H), 2.03(m, 2H), 2.13(m, 1H), 2.39(m, 1H), 3.13(dd, 1H), 4.43(dd, 1H), 3.89(m, 1H), 4.05(m, 1H), 5.92(m, 1H), 7.37(m, 1H), 7.47(m, 2H), 7.62(m, 2H), |
| # 7.91 (m, 1H); MS ES+ m/z 493 [MNa]⁺ | | | | |

### EXAMPLE 15

(2S)-2-{1-[(1S)-1-Carboxymethyl-2-(5-phenyl-oxazol-2-yl)-ethylcarbamoyl]-cyclopentylmethyl}-4-methoxy Butyric Acid

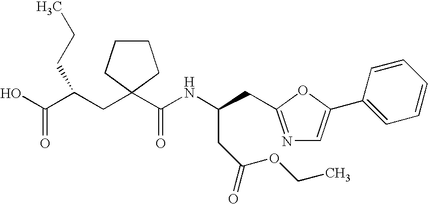

This compound was prepared by the method described in example 1 using the di-ester of preparation 100 as a starting material. ¹HNMR (CDCl₃, 400 MHz): 0.77 (t, 3H), 1.13 (m, 3H), 1.22-1.86 (m, 15H), 1.97 (m, 1H), 2.60 (m, 2H), 2.99 (m, 2H), 4.04 (m, 1H), 5.59 (m, 1H), 7.31 (m, 1H), 7.40 (m, 2H), 7.47 (m, 2H), 7.65 (m, 2H); MS ES+ m/z 485 [MH]⁺

### EXAMPLE 16

(2S)-2-{1-[(1S)-1-Carboxy-2-(5-phenyl-oxazol-2-yl)-ethylcarbamoyl]-cyclopentylmethyl}-4-methoxy-butyric Acid Propyl Ester

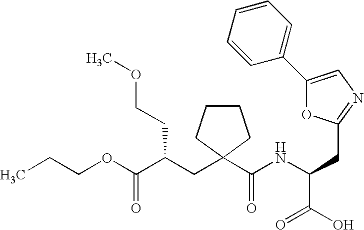

The di-ester of preparation 101 (0.34 g, 0.61 mmol) was added to a solution of trifluoroacetic acid (2 mL) in dichloromethane (10 mL) and the reaction mixture stirred at room temperature for 2 days. The reaction mixture was concentrated in vacuo, dissolved in dichloromethane (20 mL), washed with water (20 mL), dried over magnesium sulphate and concentrated in vacuo to yield the title product; ¹HNMR(CDCl₃, 400 MHz): 0.93 (t, 3H), 1.44-1.76 (m, 9H), 1.83 (m, 2H), 2.08 (m, 3H), 2.51 (m, 1H), 3.25 (s, 3H), 3.36 (m, 2H), 3.42 (m, 2H), 3.61 (m, 1H), 3.98 (m, 1H), 4.92 (m, 1H), 7.18 (m, 1H), 7.32-7.44 (m, 4H), 7.60 (m, 2H); MS ES− m/z 499 [M−H]⁻

The following compounds were made by the method described above using the appropriate ^{t}butyl ester starting material.

### EXAMPLE 17

(2S)-2-{1-[(1S)-1-Carboxy-2-(5-phenyl-oxazol-2-yl)-ethylcarbamoyl]-cyclopentylmethyl}-4-methoxy-butyric Acid Butyl Ester

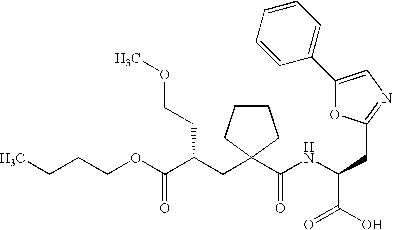

The title compound was prepared following a method similar to that in example 16, using the di-ester of preparation 102 as a starting material; ¹HNMR(CDCl₃, 400 MHz): 0.90 (t, 3H), 1.39 (m, 2H), 1.47-1.77 (m, 9H), 1.82 (m, 2H), 2.00-2.19 (m, 3H), 2.53 (m, 1H), 3.25 (s, 3H), 3.29 (m, 3H), 3.66 (m, 1H), 4.02 (m, 2H), 4.89 (m, 1H), 7.23-7.44 (m, 4H), 7.63 (m, 2H); MS ES− m/z 513 [M−H]⁻

### EXAMPLE 18

(2S)-2-{1-[(1S)-1-Cyclopentyloxycarbonyl-2-(5-phenyl-oxazol-2-yl)-ethylcarbamoyl]-cyclopentylmethyl}-4-methoxy-butyric Acid

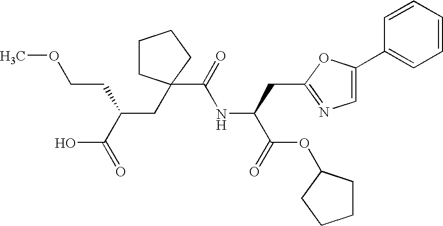

Trifluoroacetic acid (1 mL) was added to a solution of the di-ester of preparation 93 (170 mg, 0.29 mmol) in dichloromethane (3 mL) and the reaction mixture stirred at room temperature for 18 hours. The reaction mixture was concentrated in vacuo and azeotroped with dichloromethane (×6) to yield the title product; ¹HNMR (DMSOD₆, 400 MHz): 1.38-1.66 (m, 14H), 1.74 (m, 3H), 1.91 (m, 2H), 2.00 (m, 1H), 2.16 (m, 1H), 3.15 (m, 3H), 3.19 (m, 3H), 3.31 (m, 1H), 4.72 (m, 1H), 5.04 (m, 1H), 7.34 (m, 1H), 7.43 (m, 2H), 7.53 (m, 1H), 7.65 (m, 1H), 8.02 (m, 1H); MS ES− m/z 525 [M−H]⁻

The following compounds were prepared by the method described in example 18 above using the appropriate tert-butyl ester starting material

### EXAMPLE 19

(2S)-2-{1-[(1S)-1-(1-Ethyl-propoxycarbonyl)-2-(5-phenyl-oxazol-2-yl)-ethylcarbamoyl]-cyclopentylmethyl}-4-methoxy-butyric Acid

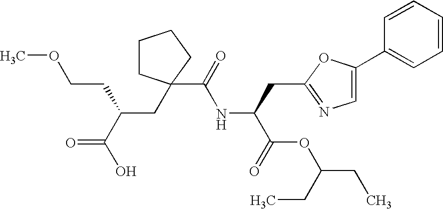

¹HNMR (DMSOD₆, 400 MHz): 0.75 (m, 6H), 1.19-1.61 (m, 11H), 1.76 (m, 2H), 1.90 (m, 3H), 2.18 (m, 1H), 3.09 (m, 1H), 3.13 (s, 3H), 3.38 (m, 3H), 4.61 (m, 1H), 4.81 (m, 1H), 7.32 (m, 1H), 7.44 (m, 2H), 7.52 (m, 1H), 7.62 (m, 2H), 8.03 (m, 1H); MS ES+ m/z 529 [MH]⁺.

### EXAMPLE 20

(2S)-2-{1-[(1S)-1-(2-Butoxy-ethoxycarbonyl)-2-(5-Phenyl-oxazol-2-yl)-ethylcarbamoyl]-cyclopentylmethyl}-4-methoxy-butyric Acid

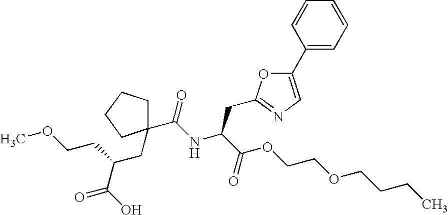

¹HNMR (CDCl₃, 400 MHz): 0.86 (t, 3H), 1.35 (m, 2H), 1.46-1.79 (m, 10H), 1.99 (m, 2H), 2.08 (m, 2H), 2.57 (m, 1H), 3.28 (s, 3H), 3.42 (m, 4H), 3.58 (m, 2H), 3.64 (m, 2H), 4.36 (m, 2H), 5.06 (m, 1H), 6.92 (m, 1H), 7.40 (m, 2H), 7.44 (m, 2H), 7.62 (m, 2H); MS ES+ m/z 559 [MH]⁺

### EXAMPLE 21

(2S)-2-{1-[(1S)-1-Ethoxycarbonyl-2-(4-phenyl-imidazol-1-yl)-ethylcarbamoyl]-cyclopentylmethyl}-4-methoxy-butyric Acid

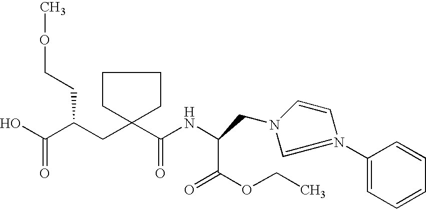

The di-ester of preparation 107 (17 mg, 0.032 mmol) was added to a solution of trifluoroacetic acid (0.5 mL) in dichloromethane (5 mL) and the reaction mixture stirred at room temperature for 18 hours. The reaction mixture was concentrated in vacuo and then azeotroped with toluene (3×15 mL), ethyl acetate (15 mL) and dichloromethane (15 mL) to yield the title product, 20 mg; ¹HNMR (CDCl₃, 400 MHz): 1.25 (m, 3H), 1.37-2.18 (m, 13H), 3.11 (s, 3H), 4.21 (m, 4H), 4.40 (m, 1H), 4.82 (m, 1H), 5.12 (m, 1H), 7.37 (m, 5H), 7.65 (m, 2H), 9.26 (m, 1H); MS ES+ m/z 486 [MH]⁺

### EXAMPLE 22

(2R)-2-{1-[(1S)-1-Butoxycarbonyl-2-(5-phenyl-oxazol-2-yl)-ethoxycarbamoyl]-cyclopentylmethyl}-pentanoic Acid

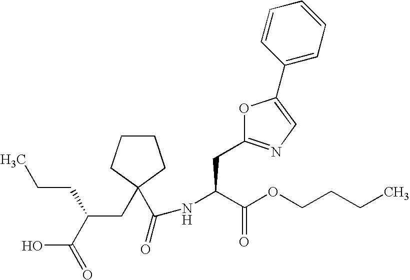

The di-ester of preparation 99 (325 mg, 0.55 mmol) was dissolved in dichloromethane (3 mL), the solution treated with trifluoroacetic acid (3 mL) and the reaction mixture stirred at room temperature for 2 hours. The reaction mixture was diluted with toluene (20 mL) and concentrated in vacuo on a cold water bath to remove trifluoroacetic acid. The reaction mixture was partitioned between ethyl acetate (25 mL) and brine (20 mL) and the organic layer dried over magnesium sulphate and concentrated in vacuo. The crude product was purified by column chromatography on silica gel eluting with pentane:ethyl acetate (75:25 to 70:30) to yield the title product, 255 mg; [α]=−11.67 (1.08 mg/mL) in methanol; ¹HNMR(CD₃OD, 400 MHz): 0.86 (m, 6H), 1.27-1.62 (m, 16H), 1.83 (m, 1H), 2.06 (m, 1H), 2.29 (m, 1H), 3.41 (m, 2H), 4.16 (m, 2H), 4.95 (m, 1H), 7.30 (m, 1H), 7.41 (m, 3H), 7.64 (m, 2H), 8.05 (m, 1H); MS ES+ m/z 499 [MH]⁺.

The following compound was made following the preparation described above and the appropriate di-ester starting material.

### EXAMPLE 23

(2R)-2-{1-[2-(5-Phenyl-oxazol-2-yl)-(1S)-1-propoxycarbonyl-ethylcarbamoyl]-cyclopentylmethyl}-pentanoic Acid

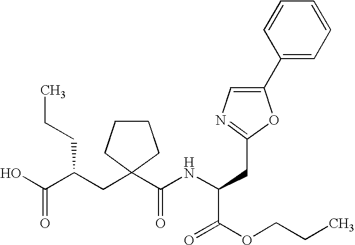

The title compound was prepared by a method similar to that described in example 22 from the di-ester of preparation 98; [α]=−16.73 (1.160 mg/mL) in methanol; ¹HNMR (CD₃OD, 400 MHz): 0.85 (t, 3H), 0.90 (t, 3H), 1.20 (m, 2H), 1.30-1.70 (m, 10H), 1.80 (m, 1H), 2.00 (m, 2H), 2.05 (m, 1H), 2.25 (m, 1H), 3.40 (m, 2H), 4.05 (m, 2H), 4.95 (m, 1H), 7.35 (t, 1H), 7.40 (s, 1H), 7.45 (t, 2H), 7.65 (d, 2H), 8.05 (d, 1H); MS ES+ m/z 485 [MH]⁺.

### EXAMPLE 24

(2S)-2-{1-[(1S)-1-Carboxy-2-(4-phenyl-imidazol-1-yl)-ethylcarbamoyl]-cyclopentylmethyl}-4-methoxy-butyric Acid

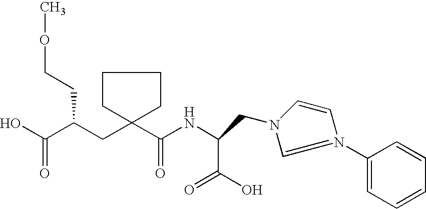

A solution of the ester of preparation 105 (17 mg, 0.033 mmol) in dichloromethane (5 mL) was treated with trifluoroacetic acid (0.5 mL) and the reaction mixture stirred at room temperature for 5 hours. The reaction mixture was concentrated in vacuo and azeotroped with toluene (3×15 mL), ethyl acetate (15 mL) and dichloromethane (10 mL) to yield the title product, 11.1 mg; ¹HNMR (CD₃OD, 400 MHz): 1.41-1.94 (m, 11H), 3.21 (m, 6H), 4.77 (m, 2H), 7.52 (m, 3H), 7.71 (m, 2H), 7.94 (m, 1H), 8.91 (m, 1H); MS ES− m/z 456 [M−H]⁻.

### EXAMPLE 25

(2S)-2-{1-[(1S)-1-Carboxy-2-(4-methyl-5-phenyl-oxazol-2-yl)-ethoxycarbamoyl]-cyclopentylmethyl}-4-methoxy-butyric Acid

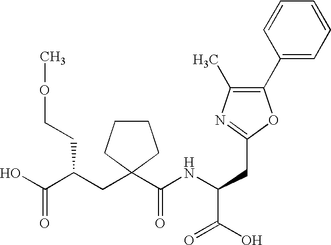

The ester of example 1 (500 mg, 1 mmol) was added to a solution of 1M sodium hydroxide (4 mL) in dioxan (12 mL) and the reaction mixture stirred at 50° C. for 5 hours and left at room temperature for a further 18 hours. The reaction mixture was concentrated in vacuo, acidified with 2M hydrochloric acid (20 mL) and extracted with ethyl acetate (2×30 mL). The organics were dried over magnesium sulphate and concentrated in vacuo. The crude product was purified by column chromatography on silica gel eluting with dichloromethane:methanol:acetic acid (90:10:1) to yield the title product; [α]=−6.54 (1.620 mg/mL) in methanol; ¹HNMR (CDCl₃, 400 MHz): 1.58 (brm, 12H), 2.06 (s, 3H), 2.18 (m, 1H), 2.48 (s, 1H), 3.22 (s, 3H), 3.36 (s, 2H), 3.48 (d, 2H), 4.94 (d, 1H), 7.32 (m, 2H), 7.42 (m, 2H), 7.57 (m, 1H); MS ES+ m/z 473[MH]⁺.

The following compounds were prepared by the method described in example 25 using the appropriate starting mono-ester.

### EXAMPLE 26

(2S)-2-(1-{(1S)-1-Carboxy-2-[5-(4-fluoro-phenyl)-oxazol-2-yl]-ethylcarbamoyl}-cyclopentylmethyl-4-methoxy-butyric Acid

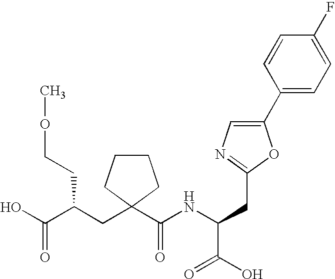

¹HNMR (CDCl₃, 400 MHz): 1.63 (brm, 12H), 2.56 (brs, 1H), 3.33 (s, 3H), 3.46 (t, 2H), 3.58-3.77 (m, 2H), 5.06 (m, 1H), 7.16 (t, 4H), 7.21 (s, 1H) 7.38 (s, 1H); MS ES− m/z 475 [M−H]⁻

### EXAMPLE 27

(2R)-2-{1-[(1S)-1-Carboxymethyl-2-(5-phenyl-oxazol-2-yl)-ethylcarbamoyl]-cyclopentylmethyl}-pentanoic Acid

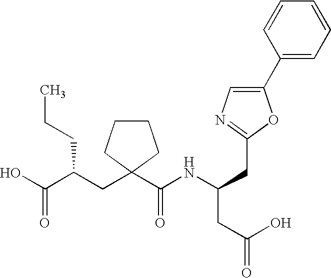

[α]=−13.13 (0.960 mg/mL) in methanol; ¹HNMR (CDCl₃, 400 MHz): 0.83 (t, 3H), 1.20-1.52 (m, 3H), 1.54-1.81 (m, 7H), 1.86 (m, 1H), 2.00-2.19 (m, 3H), 2.36 (m, 1H), 2.53 (dd, 1H), 2.66 (dd, 1H), 3.38 (m, 1H), 3.60 (m, 1H), 4.94 (m, 1H), 7.22 (m, 1H), 7.36 (m, 1H), 7.38 (m, 2H), 7.61 (m, 3H); MS ES− m/z 455 [M−H]⁻.

### EXAMPLE 28

(2S)-2-{1-[(1S)-1-Carboxy-2-(3-phenyl-[1,2,4]oxadiazol-5-yl)-ethylcarbamoyl]-cyclopentylmethyl}-4-methoxy-butyric Acid

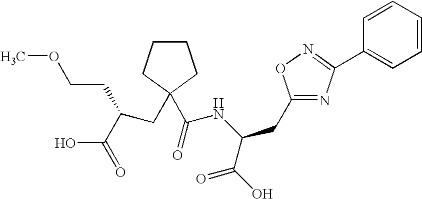

A solution of the ester of example 42 (50 mg, 0.10 mmol) in dioxan (1.5 mL) was treated with 2M sodium hydroxide solution (1.5 mL) and the reaction mixture stirred at room temperature for 18 hours. The reaction mixture was acidified with 2M hydrochloric acid and extracted with ethyl acetate (30 mL). The organic layer was separated and washed with brine, dried over magnesium sulphate and concentrated in vacuo. The product was azeotroped with ether before being purified by column chromatography on silica gel eluting with dichloromethane:methanol:acetic acid (100:0:0 to 90:10:1). The appropriate fractions were combined and concentrated in vacuo before being azeotroped with toluene and ether to yield the title product, 24 mg; [α]=−7.19 (1.03 mg/mL) in methanol; ¹HNMR (acetone, 400 MHz): 1.38-1.63 (m, 10H), 1.83 (m, 2H), 2.43 (m, 1H), 3.16 (s, 3H), 3.34 (m, 3H), 3.59 (m, 1H), 5.13 (m, 1H), 7.57 (m, 4H), 8.08 (m, 2H), MS ES− m/z 458 [M−H]⁻.

### EXAMPLE 29

(2R)-2-{1-[(1S)-1-Carboxy-2-(5-phenyl-oxazol-2-yl)-ethylcarbamoyl]-cyclopentylmethyl}-pentanoic Acid

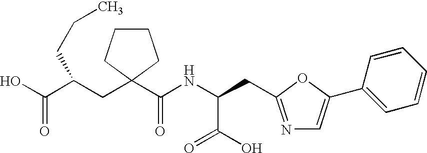

The ester of example 4 (130 mg, 0.2 mmol) was added to a solution of 1M sodium hydroxide (6 mL) in methanol (9 mL) and the reaction mixture stirred at room temperature for 18 hours. The reaction mixture was concentrated in vacuo, diluted with water (15 mL) and the aqueous washed with ethyl acetate (15 mL). The aqueous layer was acidified to pH 1 with 2M hydrochloric acid and extracted into ethyl acetate (3×20 mL). These organics were combined, dried over magnesium sulphate and concentrated in vacuo. The crude product was purified by column chromatography on silica gel eluting with dichloromethane:methanol 100:0 to 95:5 to yield the title product; [α]=−14.0 (0.70 mg/mL) in methanol; ¹HNMR (CDCl₃, 400 MHz): 0.94 (t, 3H), 1.19-1.64 (m, 13H), 2.11 (m, 1H), 2.36 (m, 1H), 3.49 (m, 2H), 4.98 (m, 1H), 7.17 (d, 1H), 7.28 (m, 2H), 7.37 (m, 3H); MS ES− m/z 441 [M−H]⁻.

The following compounds were made by a similar method to that described in example 29 using the appropriate alkyl ester starting material.

### EXAMPLE 30

(2S)-2-(1-{(1S)-1-Carboxy-2-[5-(4-chloro-phenyl)-oxazol-2-yl]-ethylcarbamoyl}-cyclopentylmethyl)-4-methoxy-butyric Acid

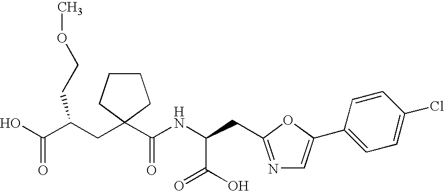

¹HNMR (CDCl₃, 400 MHz): 1.47-1.72 (brm, 8H), 1.91 (m, 2H), 2.18 (m, 2H), 2.54 (m, 1H), 3.27 (s, 3H), 3.36 (brs, 2H), 3.47 (m, 2H), 4.97 (d, 1H), 7.15 (d, 1H), 7.24 (s, 1H), 7.39 (d, 2H), 7.54 (d, 2H); MS APCI m/z 493 [MH]⁺

### EXAMPLE 31

(2S)-2-{1-[(1S)-1-Carboxy-2-(4-phenyl-oxazol-2-yl)-ethylcarbamoyl]-cyclopentylmethyl}-4-methoxy-butyric Acid

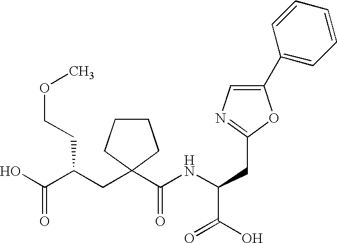

[α]=+3.08 (0.91 mg/mL) in methanol; ¹HNMR (CD₃OD, 400 MHz): 1.31 (m, 1H), 1.51-1.93 (m, 8H), 2.03 (m, 3H), 2.44 (m, 1H), 3.21 (s, 3H), 3.29 (m, 2H), 3.42 (m, 2H), 5.13 (m, 1H), 7.23 (m, 1H), 7.36 (m, 2H), 7.72 (m, 2H), 8.13 (m, 1H); MS ES− m/z 457 [MH]⁻.

### EXAMPLE 32

(2R)-2-{1-[(1S)-1-Carboxy-2-(2-phenyl-oxazol-5-yl)ethylcarbamoyl]-cyclopentylmethyl}-pentanoic Acid

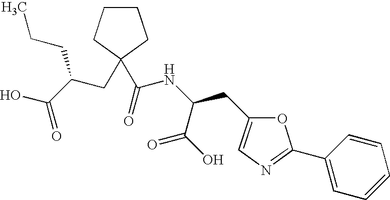

2M Sodium hydroxide (5 mL) was added to a solution of the ester of example 6 (150 mg, 0.31 mmol) in tetrahydrofuran (5 mL) and the reaction mixture heated under reflux for 3 hours. The reaction mixture was concentrated in vacuo, acidified with 2M hydrochloric acid (10 mL) and extracted into ethyl acetate (2×25 mL). The organics were combined, washed with brine (25 mL), dried over magnesium sulphate and concentrated in vacuo. The crude product was purified by column chromatography on silica gel eluting with dichloromethane:methanol (95:5), the appropriate fractions were combined and concentrated in vacuo to yield the title product, 120 mg; [α]=−30.01 (1.02 mg/mL) in methanol; ¹HNMR (CDCl₃, 400 MHz): 0.82 (m, 3H), 1.27 (m, 3H), 1.64 (m, 8H), 2.11 (m, 1H), 2.16 (m, 1H), 2.31 (m, 1H), 2.40 (s, 1H), 3.18 (dd, 1H), 3.22 (dd, 1H) 4.82 (d, 1H), 7.46 (s, 3H), 7.62 (s, 2H), 8.04 (m, 2H); MS APCI m/z 443 [MH]⁺

### EXAMPLE 33

(2R)-2-{1-[(1S)-1-Carboxy-2-(5-phenyl-oxazol-4-yl)-ethylcarbamoyl]-cyclopentylmethyl}-pentanoic Acid

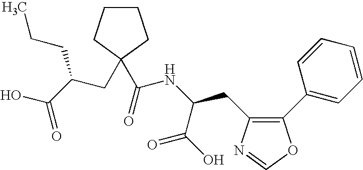

The title compound was prepared by a method similar to that described in example 32 using the ester of example 14 as a starting material; ¹HNMR (CDCl₃, 400 MHz): 0.85 (t, 3H), 1.30 (m, 3H), 1.60 (m, 8H), 2.00 (m, 2H), 2.20 (m, 1H), 2.40 (brm, 1H), 3.30 (m, 1H), 3.45 (m, 1H), 4.85 (m, 1H), 7.35 (m, 1H), 7.45 (m, 3H), 7.60 (d, 2H), 7.90 (s, 1H); MS ES− m/z 441 [MH]⁺.

### EXAMPLE 34

(2R)-2-{1-[(1S)-1-Carboxy-2-(4-phenyl-oxazol-2-yl)-ethylcarbamoyl]-cyclopentylmethyl}-pentanoic Acid

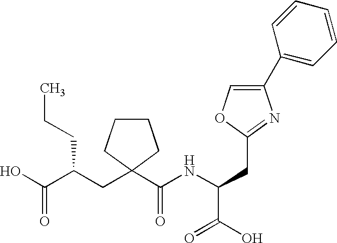

The di-ester of preparation 78 (200 mg, 0.43 mmol) was added to a solution of trifluoroacetic acid (1 mL) in dichloromethane (4 mL) and the reaction mixture stirred at room temperature for 5 hours. The reaction mixture was concentrated in vacuo and treated with 2M sodium hydroxide solution (2 mL) in dioxan (4 mL) and stirred at 50° C. for 5 hours. The reaction mixture was concentrated in vacuo and the residue diluted with ethyl acetate (20 mL) and 2M hydrochloric acid (10 mL). The organic layer was separated and dried over magnesium sulphate and concentrated in vacuo. The crude product was purified by column chromatography on silica gel eluting with dichloromethane:methanol 95:5 to yield the title product, 90 mg; ¹HNMR (CDCl₃, 400 MHz): 0.85 (t, 3H), 1.02-1.49 (m, 11H), 1.77 (m, 1H), 1.88 (m, 1H), 2.01 (m, 1H), 2.16 (m, 1H), 3.19 (dd, 1H), 3.30 (dd, 1H), 5.04 (m, 1H), 7.24 (m, 1H), 7.36 (m, 2H), 7.71 (m, 2H), 7.89 (m, 1H), 8.63 (m, 1H); MS ES+ m/z 441 [MH]⁺.

The following products were prepared by a method similar to that described in example 34 using the appropriate di-ester starting material.

### EXAMPLE 35

(2S)-2-{1-[(1S)-1-Carboxy-2-(5-phenyl-[1.2.4]oxadiazol-3-yl)-ethylcarbamoyl]-cyclopentylmethyl}-4-methoxy-butyric Acid

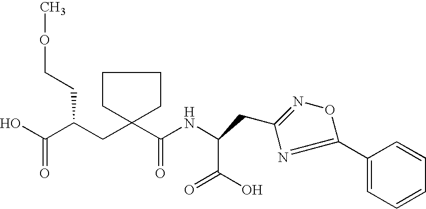

¹HNMR (CDCl₃, 400 MHz): 1.68 (m, 8H), 1.84 (m, 1H), 2.07 (m, 2H), 2.28 (m, 1H), 2.57 (m, 1H), 3.28 (s, 3H), 3.32-3.54 (brm, 4H), 4.96 (m, 1H), 7.17 (d, 1H), 7.54 (m, 2H), 7.60 (m, 1H), 8.09 (m, 2H); MS ES+ m/z 460 [MH]⁺.

### EXAMPLE 36

(2R)-2-{1-[(1S)-1-carboxy-2-(4-phenyl-pyrazol-1-yl)-ethylcarbamoyl]-cyclopentylmethyl}-pentanoic Acid

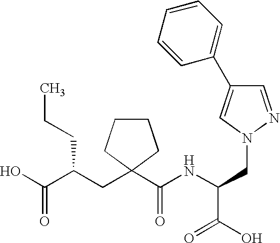

[α]=+16.83 (0.820 mg/mL) in methanol; ¹HNMR (CDCl₃, 400 MHz): 1.18-2.20 (m, 18H), 4.91 (m, 1H), 5.07 (m, 2H), 7.23 (m, 1H), 7.42 (m, 4H), 7.57 (m, 1H), 8.19 (m, 1H), 8.26 (m, 1H); MS ES− m/z 440 [MH]⁻.

### EXAMPLE 37

(2R)-2-{1-[(1S)-1-Carboxy-2-(4-isobutyl-oxazol-2-yl)-ethylcarbamoyl]-cyclopentylmethyl}-pentanoic Acid

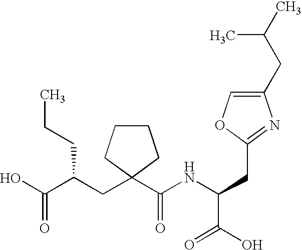

[α]=−1.9 (1.06 mg/mL) in methanol; ¹HNMR (CDCl₃, 400 MHz): 0.86 (m, 9H), 1.20-1.42 (m, 3H), 1.44-1.71 (m, 9H), 1.96-2.17 (m, 3H), 2.34 (m, 3H), 3.41 (m, 2H), 4.96 (m, 1H), 7.20 (m, 1H), 7.36 (m, 1H); MS ES+ m/z 421 [MH]⁺

### EXAMPLE 38

(2R)-2-{1-[(1S)-1-Carboxy-2-(4-ethyl-oxazol-2-yl)-ethylcarbamoyl]-cyclopentylmethyl}-pentanoic Acid

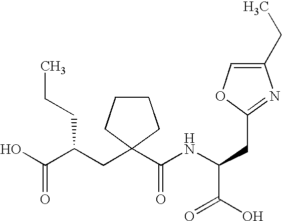

Prepared as described in example 34 but the reaction was stirred with sodium hydroxide and dioxan for 7 hours at 80° C.; [α]=−4.0 (1.75 mg/mL) in methanol; ¹HNMR (CDCl₃, 400 MHz): 0.86 (t, 3H), 1.17 (t, 3H), 1.24-1.73 (m, 12H), 1.96-2.21 (m, 3H), 2.52 (m, 2H), 3.39 (m, 2H), 4.85 (m, 1H), 7.17 (m, 1H), 7.29 (m, 1H); MS ES− m/z 393 [MH]⁺.

### EXAMPLE 39

(2S)-2-{1-[(1S)-1-Carboxy-2-(5-phenyl-[1.3.4]oxadiazol-2-yl)-ethylcarbamoyl]-cyclopentylmethyl}-4-methoxy-butyric Acid

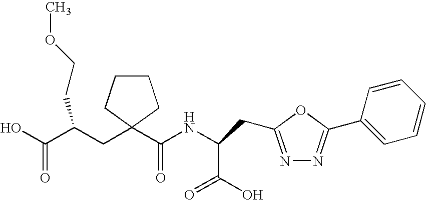

The di-ester of preparation 89 (80 mg, 0.2 mmol) was dissolved in dichloromethane (3 mL), the reaction mixture treated with trifluoroacetic acid (0.5 mL) and then stirred at room temperature for 18 hours. The reaction mixture was concentrated in vacuo and azeotroped with toluene. The residue was purified by column chromatography on silica gel eluting with dichloromethane:methanol:acetic acid 97:3:0.5 to yield a colourless solid. The solid was dissolved in methanol (3 mL) and treated with 2M sodium hydroxide solution (1 mL). The reaction mixture was stirred at room temperature for 18 hours. The reaction mixture was concentrated in vacuo and the residue partitioned between 2M hydrochloric acid (10 mL) and ethyl acetate (10 mL). The layers were separated and the aqueous layer extracted with ethyl acetate (2×10 mL) The combined organic extracts were dried over magnesium sulphate and concentrated in vacuo. The crude product was purified by column chromatography on silica gel eluting with dichloromethane:methanol:acetic acid 95:5:0.5 to yield the title product, 30 mg; [α]=−7.40 (1.00 mg/mL) in methanol; ¹HNMR (CD₃OD, 400 MHz): 1.38-1.83 (m, 9H), 1.96-2.17 (m, 3H), 2.39 (m, 1H), 3.24 (s, 3H), 3.29 (m, 3H), 3.48 (m, 1H), 3.57 (m, 1H), 5.10 (m, 1H), 7.54 (m, 3H), 8.02 (m, 2H); MS ES+ m/z 460.2 [MH]⁺.

### EXAMPLE 40

(2S)-2-{1-[(1S)-1-Carboxy-2-(5-phenyl-oxazol-2-yl)-ethylcarbamoyl]-cyclopentylmethyl}-4-methoxy-butyric Acid

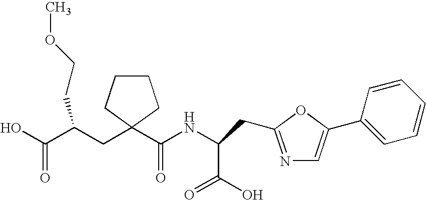

The di-ester of preparation 80 (200 mg, 0.36 mmol) was added to a solution of 1M sodium hydroxide (7 mL) in methanol (8 mL) and the reaction mixture stirred at room temperature for 18 hours. The reaction mixture was concentrated in vacuo, diluted with water and washed with ethyl acetate (15 mL). The aqueous layer was acidified with 2M hydrochloric acid and the white precipitate extracted into ethyl acetate (3×15 mL). The organics were combined, dried over magnesium sulphate and concentrated in vacuo. The product was added to a solution of trifluoroacetic acid in dichloromethane and the reaction mixture stirred at room temperature for 18 hours. The reaction mixture was concentrated in vacuo and azeotroped with toluene. The crude product was purified by column chromatography on silica gel, eluting with dichloromethane:methanol 100:0 to 95:5 to to yield the title product, 132 mg; [α]=−6.00 (0.80 mg/mL) in methanol; ¹HNMR (CDCl₃, 400 MHz): 1.61 (br, 9H), 1.82-2.24 (brm, 4H), 3.26 (s, 3H), 3.40 (s, 2H), 3.58 (s, 2H), 5.01 (s, 1H), 7.19 (s, 1H), 7.21-7.43 (m, 4H), 7.61 (m, 2H); MS ES− m/z 457 [M−H]⁻.

### EXAMPLE 41

(2R)-2-{1-[(1S)-1-Carboxy-2-(3-phenyl-[1,2,4]oxadiazol-5-yl)-ethylcarbamoyl]-cyclopentylmethyl}-pentanoic Acid

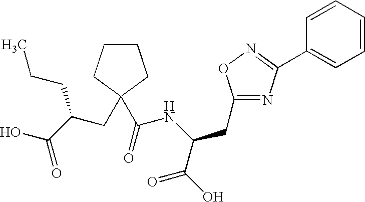

A solution of the di-ester of preparation 90 (170 mg, 0.29 mmol) in dioxan (2 mL) was treated with 2M sodium hydroxide solution (2 mL) and the reaction mixture stirred at room temperature for 18 hours. The reaction mixture was acidified with 0.5M hydrochloric acid until pH 1 and was extracted with ethyl acetate (1×80 mL, 1×20 mL). The organics were combined, washed with brine, dried over magnesium sulphate and concentrated in vacuo. The crude product was azeotroped with toluene before being dissolved in dichloromethane (2 mL). The reaction mixture was treated with trifluoroacetic acid (1 mL) and stirred at room temperature for 40 minutes, additional trifluoroacetic acid (1 mL) was added and the reaction mixture stirred for an extra 30 minutes. The reaction mixture was concentrated in vacuo and azeotroped with toluene. The crude product was purified by column chromatography on silica gel eluting with dichloromethane:methanol:acetic acid (97.5:2.5:0.5) to yield the title product, 19 mg; [α]=−16.55 (1.10 mg/mL) in methanol; ¹HNMR (CDCl₃, 400 MHz): 0.81 (t, 3H), 1.13-1.64 (m, 10H), 1.92 (m, 1H), 1.97-2.17 (m, 3H), 2.29 (m, 1H), 3.49 (dd, 1H), 3.62 (dd, 1H), 4.99 (m, 1H), 7.46 (m, 3H), 8.04 (m, 2H); MS ES− m/z 442 [M−H]⁻.

### EXAMPLE 42

(2S)-2-{1-[(1S)-1-Carboxy-2-(3-phenyl-[1.2.4]oxadiazol-5-yl)-ethylcarbamoyl]-cyclopentylmethyl}-4-methoxy-butyric Acid Ethyl Ester

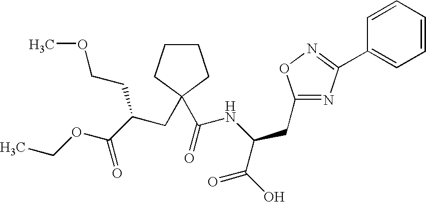

The di-ester of preparation 104 (90 mg) was added to a solution of 10% Pd/C (10 mg) in ethanol (5 mL) and the reaction mixture stirred at room temperature under 50 psi of hydrogen for 2 hours. The reaction mixture was filtered through Arbocel, washing through with ethanol and concentrated in vacuo. The residue was purified by column chromatography on silica gel eluting with pentane:ethyl acetate 70:30 and then again by column chromatography on silica gel eluting with dichloromethane:methanol:acetic acid 95:5:0.5. The crude product was heated in 10% potassium carbonate solution and pentane:ethyl acetate 90:10 added. The reaction mixture was filtered through cotton wool and the aqueous layer separated. The aqueous layer was acidified with 2M hydrochloric acid and extracted with ethyl acetate (10 mL). The organic layer was dried over magnesium sulphate, filtered and concentrated in vacuo to yield the title product; ¹HNMR(CDCl₃, 400 MHz): 1.20 (t, 3H), 1.39-1.69 (m, 7H), 1.82 (m, 2H), 2.09 (m, 3H), 2.47 (m, 1H), 3.24 (s, 3H), 3.48 (m, 3H), 3.62 (m, 3H), 4.04 (m, 2H), 5.06 (m, 1H), 7.23 (m, 2H), 7.48 (m, 3H), 8.01 (m, 1H); MS ES+ m/z 488 [MH]⁺.

Preparation 1

(2S)-2-Benzyloxycarbonylamino-succinic Acid 1-ethyl Ester

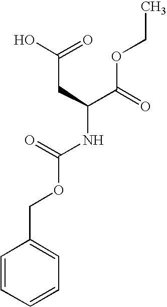

A solution of N-carbobenzyloxy-L-aspartic anhydride (100 g) in ethanol (600 mL) was refluxed for 18 hours. The reaction mixture was concentrated in vacuo, redissolved in ethyl acetate (250 mL) and extracted into saturated sodium hydrogencarbonate solution (2×200 mL). The aqueous was acidified with 2M hydrochloric acid and the reaction mixture extracted into ethyl acetate (3×200 mL). The organics were combined, dried over magnesium sulphate and concentrated in vacuo. The product was dissolved in ether (500 mL) and dicyclohexylamine (92 mL) was added to form a precipitate which was collected by filtration. The solid was redissolved in the minimum volume of warm ethanol and stirred until a precipitate formed which was collected by filtration. The solid was then recrystallised once more from ethanol. The product was suspended in ether and washed with 2M sulphuric acid (2×100 mL). The organic solution was dried over magnesium sulphate and concentrated in vacuo to yield the title product, 22 g. Re-recrystallisation of the mother liquors from ethanol yielded a further 70 g; ¹HMMR (DMSOD₆, 400 MHz): 1.16 (s, 3H), 2.57 (d, 1H), 2.65 (d, 1H), 4.06 (d, 2H), 4.39 (m, 1H), 5.02 (s, 2H), 7.11 (m, 5H), 7.71 (d, 1H).

Preparation 2

Aziridine-1,2-dicarboxylic Acid (2S)-2-benzyl Ester 1-tert-butyl Ester

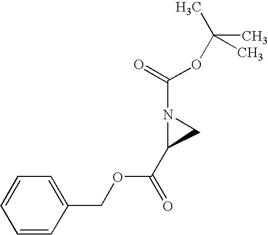

Aziridine-(2S)-2-carboxylic acid benzyl ester (Bull. Chem. Soc. Japan 51(5) 1577-1578 compound 3) (373 mg, 2.12 mmol) was added to a solution of di-tert-butyl-dicarbonate (508 mg, 2.33 mmol), triethylamine (589 μL, 4.23 mmol) and 4-dimethylaminopyridine (a few crystals) in dichloromethane (20 mL) and the reaction mixture stirred at room temperature for 7 hours. The reaction mixture was washed with 2M hydrochloric acid (20 mL), dried over magnesium sulphate and purified by column chromatography on silica gel eluting with pentane:ethyl acetate (90:10) to yield the title product, 394 mg.

¹HNMR (CDCl₃, 400 MHz): 1.46 (s, 9H), 2.43 (m, 1H), 2.56 (m, 1H), 3.08 (m, 1H), 5.24 (m, 2H), 7.39 (m, 5H); MS ES+ m/z 300 [MNa]⁺.

Preparation 3

3,6-Diethoxy-(2R)-2-isopropyl-2,5-dihydro-pyrazine

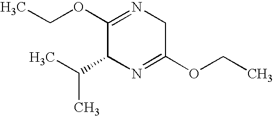

Boron trifluoride diethyl ether complex (75.4 g, 396.6 mmol) was added to a flask under a blanket of nitrogen gas, washed with ether (200 mL and 100 mL) and dissolved in dichloromethane (600 mL). R-(−)-3-isopropyl-2,5-piperazinedione (22.5 g, 144 mmol) was added portionwise to the flask and the reaction mixture stirred at room temperature for 48 hours. The reaction mixture was poured slowly into a stirred suspension of sodium hydrogencarbonate powder (100 g) in iced water (1000 mL). The organic layer was separated, washed with brine (100 mL), dried over magnesium sulphate and concentrated in vacuo. The crude product was purified by column chromatography on silica gel eluting with pentane:ethyl acetate (60:40) to yield the title product, 29.4 g; ¹HNMR (CDCl₃, 400 MHz): 0.80 (d, 3H), 1.05 (d, 3H), 1.30 (m, 6H), 2.25 (m, 1H), 4.00 (m, 3H), 4.10 (m, 3H), 4.20 (m, 1H); MS ES+ m/z 213 [MH]⁺.

Preparation 4

1-[(2S)-2-(tert-Butoxycarbonyl)-4-methoxy-butyl]-cyclopentanecarboxylic Acid Benzyl Ester

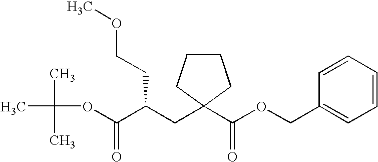

1-[(2S)-2-(tert-Butoxycarbonyl)-4-methoxybutyl]cyclopentanecarboxylic acid (6.0 g, 20 mmol) (WO0279143, page 71, preparation 69) was dissolved in N,N-dimethylformamide (20 mL) and the reaction mixture treated with caesium carbonate (8.02 g, 24.6 mmol) and stirred for 30 minutes at room temperature. Benzyl bromide (2.92 mL, 24.6 mmol) was added and the reaction mixture stirred under nitrogen at room temperature for 18 hours. The reaction mixture was diluted with ethyl acetate (150 mL) and washed with water (2×100 mL) and saturated sodium hydrogencarbonate solution (100 mL). The organic layer was dried over magnesium sulphate and concentrated in vacuo to yield the title product, 8.2 g; ¹HNMR (CDCl₃, 400 MHz): 1.24 (s, 9H), 1.43-1.69 (m, 8H), 1.82 (m, 2H), 2.14 (m, 3H), 2.36 (m, 1H), 3.25 (m, 4H), 5.14 (m, 2H), 7.38 (m, 5H); MS ES+ m/z 413 [MNa]⁺.

Preparation 5

1-[(2S)-(2-carboxy-4-methoxy-butyl)-cyclopentanecarboxylic]acid Benzyl Ester

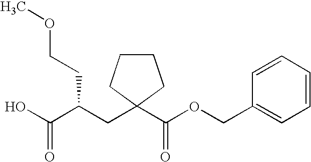

A solution of the tert-butyl ester of preparation 4 (8.19 g, 20 mmol) in dichloromethane (80 mL) was treated portionwise with trifluoroacetic acid (8 mL) and the reaction mixture stirred at room temperature for 72 hours. The reaction mixture was diluted with toluene (100 mL) and concentrated in vacuo before being azeotroped with toluene, ethyl acetate and ether to yield the title product, 6.8 g; ¹HNMR (CDCl₃, 400 MHz): 1.51 (m, 2H), 1.62 (m, 6H), 1.85 (m, 3H), 2.12 (m, 1H), 2.39 (m, 1H) 3.24 (s, 3H), 3.36 (m, 2H), 5.08 (dd, 1H), 5.15 (dd, 1H), 7.36 (m, 5H); MS ES+ m/z 357 [MNa]⁺.

Preparation 6

1-[4-Methoxy-(2S)-2-Propoxycarbonyl-butyl]-cyclopentanecarboxylic Acid Benzyl Ester

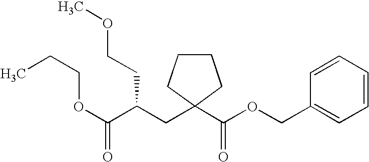

A solution of the carboxylic acid of preparation 5 (3.8 g, 11.4 mmol) in N,N-dimethylformamide (12 mL) was treated with caesium carbonate (4.5 g, 13.7 mmol) and the reaction mixture stirred at room temperature for 1 hour. 1-bromo-propane (1.25 mL, 13.8 mmol) was added and the reaction mixture stirred at room temperature for 48 hours. The reaction mixture was diluted with ethyl acetate (100 mL), washed with water (3×1 50 mL), dried over magnesium sulphate and concentrated in vacuo to yield the title product, 3.95 g; ¹HNMR (CDCl₃, 400 MHz): 0.94 (t, 3H), 1.41-1.69 (m, 9H), 1.84 (m, 2H), 2.16 (m, 3H), 2.43 (m, 1H), 3.22 (m, 5H), 3.99 (m, 2H), 5.12 (m, 2H), 7.35 (m, 5H); MS ES+ m/z 399 [MNa]⁺.

The following compounds were prepared from the acid of preparation 5 by the method of preparation 6 using the appropriate alkyl halide.

Preparation 7

1-[(2S)-(2-Butoxycarbonyl-4-methoxy-butyl)]-cyclopentanecarboxylic Acid Benzyl Ester

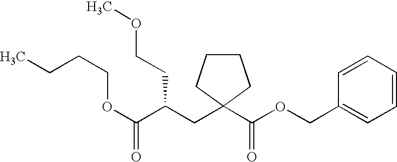

¹HNMR (CDCl₃, 400 MHz): 0.95 (t, 3H), 1.42-1.64 (m, 11H), 1.83 (m, 2H), 2.11 (m, 3H), 2.43 (m, 1H), 3.22 (m, 5H), 4.04 (m, 2H), 5.02 (dd, 1H), 5.14 (dd, 1H), 7.36 (m, 5H); MS ES+ m/z 413 [MNa]⁺.

Preparation 8

1-[(2S)-(2-Ethoxycarbonyl-4-methoxy-butyl)]-cyclopentanecarboxylic Acid Benzyl Ester

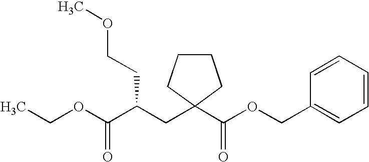

¹HNMR (CDCl₃, 400 MHz): 1.27 (m, 3H), 1.42-1.79 (m, 9H), 1.86 (m, 2H), 2.09 (m, 4H), 2.44 (m, 1H), 3.27 (s, 3H), 4.11 (m, 2H), 5.08 (dd, 1H), 5.14 (dd, 1H), 7.36 (m, 5H); MS ES+ m/z 385 [MNa]⁺.

Preparation 9

1-(4-Methoxy-(2S)-2-propoxycarbonyl-butyl)-cyclopentane Carboxylic Acid

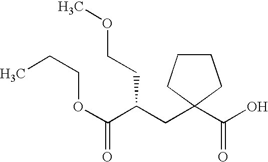

10% Pd/C (100 mg) was added to a solution of the benzyl ester of preparation 6 (1.0 g, 2.7 mmol) in tetrahydrofuran (5 mL) and the reaction mixture stirred under 50 psi hydrogen for 48 hours. The reaction mixture was filtered through Arbocel®, washing through with dichloromethane, and concentrated in vacuo to yield the title product, 0.77 g; ¹HNMR (CDCl₃, 400 MHz): 0.96 (t, 3H), 1.24-1.92 (m, 10H), 2.14 (m, 4H), 2.56 (m, 1H), 3.30 (s, 3H), 3.38 (m, 2H), 3.98 (m, 2H); MS ES+ m/z 309 [MNa]⁺.

The following compounds were prepared by a similar method to that described in preparation 9 using the appropriate benzyl ester starting material.

Preparation 10

1-[(2S)-(2-Butoxycarbonyl-4-methoxy-butyl)]-cyclopentanecarboxylic Acid

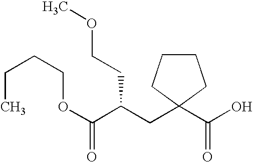

¹HNMR (CDCl₃, 400 MHz): 0.90 (t, 3H), 1.40 (m, 3H), 1.52-1.77 (m, 8H), 1.83 (m, 2H), 2.15 (m, 4H), 3.27 (s, 3H), 3.36 (m, 2H), 4.01 (m, 2H); MS ES+ m/z 323 [MNa]⁺.

Preparation 11

1-[(2S)-(2-Ethoxycarbonyl-4-methoxy-butyl)]-cyclopentanecarboxylic Acid

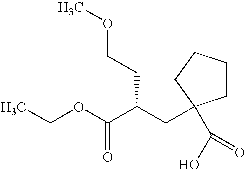

This compound was additionally purified by column chromatography on silica gel eluting with pentane:ethyl acetate:acetic acid 80:20:1 to yield the title product; [α]=−12.97; ¹HNMR (CDCl₃, 400 MHz): 1.24 (t, 3H), 1.43 (m, 1H), 1.56 (m, 1H), 1.63-1.78 (m, 5H), 1.81-1.93 (m, 2H), 2.10 (m, 5H), 2.53 (m, 1H), 3.06 (s, 3H), 4.09 (m, 2H); MS ES+ m/z 295 [MNa]⁺.

Preparation 12

(2S)-2-Benzyloxycarbonylamino-N-(1-methyl-2-oxo-2-phenyl-ethyl)-succinamic Acid Ethyl Ester

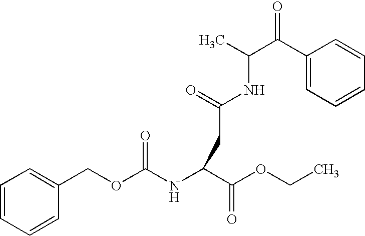

Thionyl chloride (4.4 mL, 60 mmol) and N,N-dimethylformamide (1 drop) were added to a solution of the acid of preparation 1 (4.22 g, 15 mmol) in dichloromethane (50 mL) and the reaction mixture stirred at room temperature for 18 hours. The reaction mixture was concentrated in vacuo and the product (1 g, 3 mmol) dissolved in dichloromethane (10 mL). To this was added a solution of 2-amino-1-phenyl-propan-1-one (EP 51234 pg17 preparation b) (3 mmol) and triethylamine (0.94 μL, 6 mmol) in dichloromethane (10 mL) which had been cooled to 0° C. The reaction mixture was stirred at room temperature for 4 hours. The reaction mixture was diluted with dichloromethane (30 mL), washed with 2M hydrochloric acid solution (40 mL) and dried over magnesium sulphate and concentrated in vacuo. The crude product was purified by column chromatography eluting with pentane:ethyl acetate (66:33 to 33:66) to yield the title product, 918 mg; ¹HNMR (CDCl₃, 400 MHz): 1.23 (m, 3H), 1.38 (d, 3H), 2.77 (m, 1H), 2.99 (m, 1H), 4.18 (m, 2H), 4.58 (brs, 1H), 5.13 (s, 2H), 5.51 (m, 1H), 5.96 (brm, 1H), 6.69 (brm, 1H), 7.32 (m, 5H), 7.45 (m, 2H), 7.59 (m, 1H), 7.92 (d, 2H); MS APCI m/z 427 [MH]⁺.

The following compounds were prepared by the method described in preparation 12 using the acid of preparation 1 and the appropriate aromatic amine.

Preparation 13

(2S)-2-Benzyloxycarbonylamino-N-[2-(4-fluoro-phenyl)-2-oxo-ethyl]-succinamic Acid Ethyl Ester

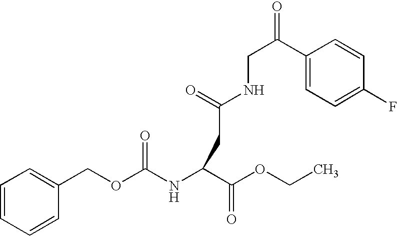

Amine used was 2-amino-1-(4-fluoro-pheny)-ethanone (U.S. Pat. No. 4,049,650 page 4 example VI A); [α]=−10.67; ¹HNMR (CDCl₃, 400 MHz): 1.21 (t, 3H), 2.85 (d, 1H), 3.02 (d, 1H), 4.20 (m, 2H), 4.61 (m, 1H), 4.65 (s, 2H), 5.14 (s, 2H), 6.00 (m, 1H) 6.68 (m, 1H), 7.16 (t, 2H), 7.32 (s, 5H), 7.94 (m, 2H); MS APCI m/z 431 [MH]⁺.

Preparation 14

(2S)-2-Benzyloxycarbonylamino-N-(2-oxo-2-phenyl-ethyl)-succinamic Acid Ethyl Ester

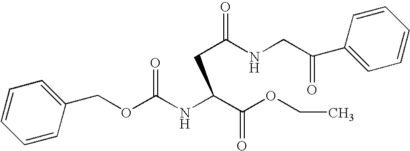

This compound was also washed with saturated sodium hydrogencarbonate solution (100 mL); ¹HNMR (CDCl₃, 400 MHz): 1.23 (m, 3H), 2.86 (d, 1H), 3.05 (d, 1H), 4.21 (m, 2H), 4.63 (s, 1H), 4.71 (s, 2H), 6.00 (s, 1H), 6.63 (s, 1H), 7.11-7.66 (brm, 8H), 7.93 (d, 2H); MS ES+ m/z 435 [MNa]⁺.

Preparation 15

4-(N′-Benzoyl-hydrazino)-(2S)-2-benzyloxycarbonylamino-4-oxo-butyric Acid Ethyl Ester

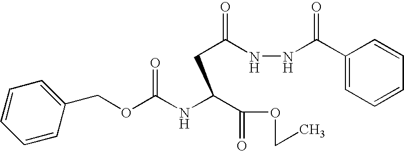

Amine used was benzoyl hydrazine (Inorganica Chemica Acta, 1995 231(1-2) 237-239)

Pyridine was used instead of triethylamine as the base and ether as the solvent for the section of the reaction performed at 0° C.; ¹HNMR (CDCl₃, 400 MHz): 1.22 (t, 3H), 2.96 (dd, 1H), 3.07 (dd, 1H), 4.13 (m, 1H), 4.22 (m, 2H), 5.10 (m, 2H), 6.09 (m, 1H), 7.27 (m, 5H), 7.42 (m, 2H), 7.58 (m, 1H), 9.03 (m, 1H), 9.16 (m, 1H); MS ES+ m/z 436 [MNa]⁺.

Preparation 16

(2S)-2-Benzyloxycarbonylamino-N-[2-(4-chloro-phenyl)-2-oxo-ethyl]-succinamic Acid Ethyl Ester

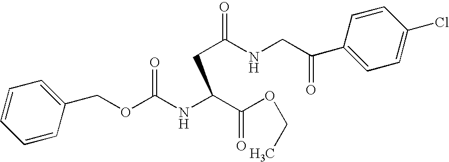

A solution of the acid of preparation 1 (9 g, 19.5 mmol) in dichloromethane (60 mL) was treated with thionyl chloride (5.5 mL, 78.3 mmol) and N,N-dimethylformamide (1 drop) and the reaction mixture stirred at room temperature for 18 hours. The reaction mixture was then concentrated in vacuo yielding a yellow oil. 2-Amino-1-(4-chloro-phenyl)-ethanone (U.S. Pat. No. 4,049,650 page 5 example VII) (4 g, 23.4 mmol) was dissolved in dichloromethane (60 mL), cooled to 0° C. and treated with triethylamine (6.5 mL, 46.8 mmol). This was added to the yellow oil and the reaction mixture stirred at room temperature for 6 hours. The reaction mixture was washed with 2M hydrochloric acid (40 mL) and saturated sodium hydrogencarbonate solution (30 mL), dried over magnesium sulphate and concentrated in vacuo to yield the title product; ¹HNMR (CDCl₃, 400 MHz): 1.26 (t, 3H), 2.84 (d, 2H), 4.20 (m, 2H), 4.62 (m, 1H), 4.70 (s, 2H), 5.08 (s, 2H), 6.59 (s, 1H), 7.21-7.39 (brm, 5H), 7.43 (d, 2H), 7.88 (d, 2H); MS ES+ m/z 469 [MNa]⁺.

Preparation 17

(3S)-3-Benzyloxycarbonylamino-4-(2-oxo-2-phenyl-ethylcarbamoyl)-butyric Acid Methyl Ester

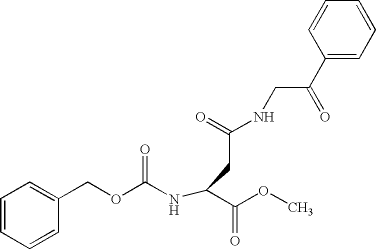

This compound was prepared by the method described in preparation 16 using □-(S)-Benzyloxycarbonylaminoglutaric acid monomethyl ester and 2-amino-1-phenyl ethanone and purifying the crude product by column chromatography on silica gel, eluting with pentane:ethyl acetate 66:33 to 33:66. The product was then triturated in ether to give the title compound; [α]=−5.46; ¹HNMR (DMSOD₆, 400 MHz): 2.41 (m, 2H), 2.58 (m, 2H) 3.54 (s, 3H), 4.18 (m, 1H), 4.55 (dd, 1H), 4.61 (dd, 1H), 4.97 (m, 2H), 7.36 (m, 5H), 7.52 (m, 2H), 7.64 (m, 1H), 7.95 (m, 2H), 8.28 (m, 1H); MS ES+ m/z 435 [MNa]⁺.

Preparation 18

(2S)-2-tert-Butoxycarbonylamino-N-((2S)-2-hydroxy-1-phenyl-ethyl)-succinamic Acid Benzyl Ester

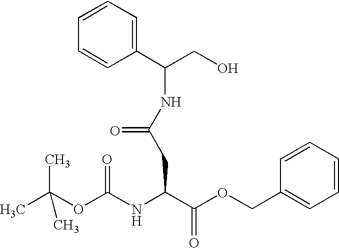

(2S)-2-Amino-2-phenyl-ethanol (0.44 g, 3.2 mmol) was added to a solution of (2S)-2-tert-butoxycarbonylamino-succinic acid-1-benzyl ester (0.87 g, 2.7 mmol), 1-(3-dimethylaminopropyl)-3-ethylcarbodiimide hydrochloride (0.52 g, 2.7 mmol), 1-hydroxybenzotriazole hydrate hydrate (0.36 g, 2.6 mmol) and 4-methylmorpholine (0.74 mL, 6.7 mmol) in dichloromethane (25 mL) and the reaction mixture stirred at room temperature for 18 hours. The reaction mixture was diluted with dichloromethane (20 mL), washed with 2M hydrochloric acid (30 mL), dried over magnesium sulphate and concentrated in vacuo. The crude product was purified by column chromatography on silica gel eluting with dichloromethane:methanol (95:5) to yield the title product, 0.77 g; ¹HNMR (DMSOD₆, 400 MHz): 1.26 (s, 9H), 2.60 (m, 2H), 3.48 (brs, 2H), 4.77 (m, 2H), 5.06 (s, 2H), 7.25 (m, 10H), 8.17-8.38 (m, 2H); MS ES+ m/z 465 [MNa]⁺.

Preparation 19

Methyl(2S)-4-({[(1Z)-amino(phenyl)methylene]amino}oxy)-2-[(tert-butoxycarbonyl)amino]-4-oxobutanoate

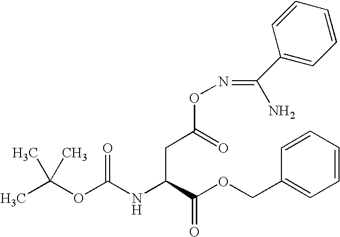

A solution of (2S)-2-tert-butoxycarbonylamino-succinic acid-1-benzyl ester (2.16 g, 6.68 mmol) in dichloromethane (10 mL) was treated with N-hydroxy-benzamidine (1 g, 7.34 mmol), 1-(3-dimethylaminopropyl)-3-ethylcarbodiimide hydrochloride (1.41 g, 7.34 mmol), 1-hydroxybenzotriazole hydrate (991 mg, 7.34 mmol) and 4-methylmorpholine (1.1 mL, 10 mmol) and the reaction mixture stirred at room temperature for 18 hours. The reaction mixture was diluted with dichloromethane (100 mL) and washed with saturated sodium hydrogen carbonate solution (50 mL) and brine (50 mL). The organic layer was dried over magnesium sulphate, concentrated in vacuo and azeotroped with ether. The solid product was triturated with diisopropyl ether (30 mL) and methanol (1.5 mL) and then recrystallised from ethyl acetate to yield the title product; ¹HNMR (CDCl₃, 400 MHz): 1.41 (s, 9H), 1.59 (m, 2H), 3.08 (dd, 1H), 3.20 (dd, 1H), 5.78 (m, 1H), 5.24 (m, 2H), 5.56 (m, 1H), 7.37 (m, 5H), 7.48 (m, 3H), 7.69 (m, 2H); MS ES+ m/z 464 [MNa]⁺.

Preparation 20

(2S)-2-Benzyloxycarbonylamino-N-(2-hydroxy-(1R)-1-phenyl-ethyl)-succinamic Acid Ethyl Ester

The title compound was prepared from the acid from preparation 1 and 2-amino-2-phenyl-ethanol following the procedure described in preparation 19.

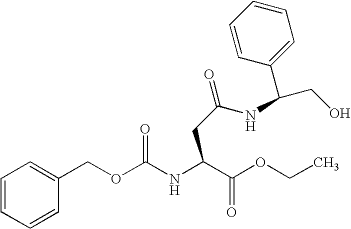

[α]=+12.45; ¹HNMR (CDCl₃, 400 MHz): 1.18 (m, 3H), 1.76-1.97 (m, 3H), 3.79 (m, 2H), 4.09 (m, 2H), 4.58 (m, 1H), 4.99 (m, 1H), 5.12 (m, 2H), 6.08 (m, 1H), 6.57 (m, 1H), 7.19-7.38 (m, 10H); MS ES+ m/z 851 [M₂Na]⁺.

Preparation 21

(2S)-2-tert-Butoxycarbonylamino-N-((1R)-1-hydroxymethyl-3-methyl-butyl)-succinamic Acid Benzyl Ester

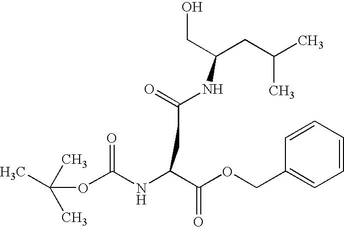

(2S)-2-tert-Butoxycarbonylamino-succinic acid 1-benzyl ester (3.43 g, 10.6 mmol) and (R)(−)leucinol (1.49 g, 12.7 mmol) were added to a solution of 1-(3-dimethylaminopropyl)-3-ethylcarbodiimide hydrochloride (2.44 g, 12.7 mmol), 1-hydroxybenzotriazole hydrate (1.75 g, 12.7 mmol) and 4-Methylmorpholine (2.9 mL, 12.7 mmol) in dichloromethane (30 mL). The reaction mixture was then stirred at room temperature for 18 hours. The reaction mixture was diluted with ethyl acetate (100 mL) and washed with 2M hydrochloric acid (100 mL), and was then dried over magnesium sulphate and concentrated in vacuo. The crude product was purified by column chromatography on silica gel eluting with dichloromethane:methanol (95:5) to yield the title product, 3.0 g. ¹HNMR (CDCl₃, 400 MHz): 0.87 (m, 6H), 1.29 (m, 2H), 1.35 (s, 9H), 1.53 (m, 1H), 2.88 (m, 2H), 3.41 (m, 1H), 3.57 (m, 1H), 3.93 (m, 1H), 4.48 (m, 1H), 5.14 (m, 2H), 5.77 (m, 2H), 7.31 (m, 5H); MS TSP+ m/z 424 [MH]⁺.

Preparation 22

(2S)-2-tert-Butoxycarbonylamino-N-((1R)-1-hydroxymethyl-propyl)-succinamic Acid Benzyl Ester

The title compound was made using (R)-2-amino-1-butanol and (2S)-2-tert-butoxycarbonylamino-succinic acid benzyl ester following the preparation described in preparation 21.

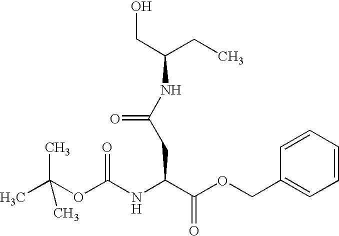

¹HNMR (CDCl₃, 400 MHz): 0.86 (t, 3H), 1.40 (s, 9H), 1.49 (m, 2H), 2.63-2.89 (m, 2H), 3.40 (m, 2H), 3.75 (m, 1H), 4.49 (m, 1H), 5.12 (m, 2H), 6.79 (m, 1H), 5.94 (m, 1H), 7.31 (m, 5H); MS ES+ m/z 417 [MNa]⁺.

Preparation 23

(2S)-2-tert-Butoxycarbonylamino-N-(2-oxo-(1R)-1-phenyl-ethyl)-succinamic Acid Benzyl Ester

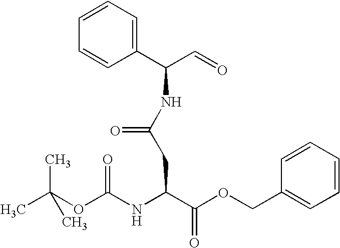

Dess Martin periodinane (2.19, 4 mmol) was added to a solution of the alcohol of preparation 18 (1.47 g, 3 mmol) in dichloromethane (20 mL), a white suspension was formed, and the reaction mixture was stirred at room temperature for 2 hours. The reaction mixture was treated with aqueous sodium thiosulphate solution (2 g in 20 mL), saturated aqueous sodium hydrogencarbonate solution (20 mL) and ether (40 mL). The reaction mixture was stirred until the solution was clear. The organics were combined, washed with brine (20 mL) dried over magnesium sulphate and concentrated in vacuo. The crude product was purified by column chromatography on silica gel, eluting with pentane:ethyl acetate 2:1 to 1:1; ¹HNMR (CDCl₃, 400 MHz): 1.42 (s, 9H), 3.31 (d, 2H), 4.84 (brs, 1H), 5.18 (d, 2H), 5.69 (brd, 1H), 7.34 (m, 2H), 7.40 (s, 3H), 7.53 (m, 2H), 7.64 (d, 2H), 7.79 (s, 1H), 8.24 (m, 2H), 9.04 (m, 1H); MS TSP+ m/z 441 [MH]⁺.

Preparation 24

(2S)-2-Benzyloxycarbonylamino-N-(2-oxo-(1R)-1-phenyl-ethyl)-succinamic Acid Ethyl Ester

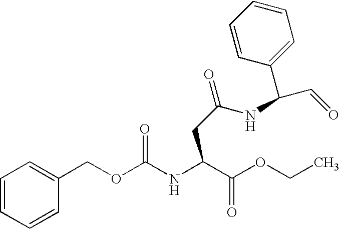

Dess Martin periodinane (2.92 g, 6.89 mmol) was added portionwise to a solution of the alcohol of preparation 20 (1.9 g, 4.6 mmol) in dichloromethane (30 mL) and the reaction mixture stirred at room temperature for 3 hours. Aqueous solutions of sodium thiosulphate (4 g in 20 mL) and sodium hydrogencarbonate (30 mL) were then added and the reaction mixture stirred until a solution was formed. Ether (100 mL) was added to the reaction mixture and the organic layer separated and washed with brine (50 mL). The reaction mixture was dried over magnesium sulphate and concentrated in vacuo to yield the title product, 1.9 g; ¹HNMR (CDCl₃, 400 MHz): 1.14 (t, 3H), 2.83 (dd, 1H), 3.03 (dd, 1H), 4.08 (m, 2H), 4.55 (m, 1H), 5.12 (m, 2H), 5.52 (m, 1H), 4.96 (m, 1H), 6.76 (m, 1H), 7.21-7.42 (m, 10H), 9.58 (m, 1H); MS ES+ m/z 435 [MNa]⁺.

The following compounds were made using a similar method to that described in preparation 24 using the appropriate starting alcohol.

Preparation 25

(2S)-2-tert-Butoxycarbonylamino-N-((1R)-1-formyl-3-methyl-butyl)-succinamic Acid Benzyl Ester

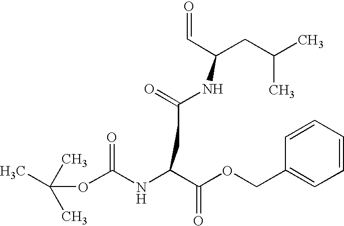

The crude product was additionally purified by column chromatography on silica gel eluting with ethyl acetate:pentane (50:50) to yield the title product; ¹HNMR (CDCl₃, 400 MHz): 0.89 (m, 6H), 1.37 (s 9H), 1.62 (m, 3H), 2.88 (m, 1H), 2.91 (m, 1H), 4.47 (m, 2H), 5.14 (m, 2H), 5.66 (m, 1H), 6.00 (m, 1H), 7.27 (m, 5H), 9.46 (m, 1H); MS TSP+ m/z 421 [MH]⁺.

Preparation 26

(2S)-2-tert-Butoxycarbonylamino-N-((1R)-1-formyl-propyl)-succinamic Acid Benzyl Ester

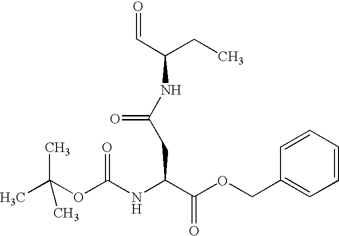

The crude product was purified by column chromatography on silica gel eluting with dichloromethane:methanol (95:5); ¹HNMR(CDCl₃, 400 MHz): 0.92 (t, 3H), 1.39 (s, 9H), 1.66 (m, 1H), 1.95 (m, 1H), 2.82 (dd, 1H), 2.96 (dd, 1H), 4.43 (m, 1H), 4.58 (m, 1H), 5.18 (m, 2H), 5.69 (m, 1H), 6.24 (m, 1H), 7.32 (m, 5H), 9.50 (m, 1H); MS TSP+ m/z 393 [MH]⁺.

Preparation 27

(2S)-2-Benzyloxycarbonylamino-3-(4-methyl-5-phenyl-oxazol-2-yl)-propionic Acid Ethyl Ester

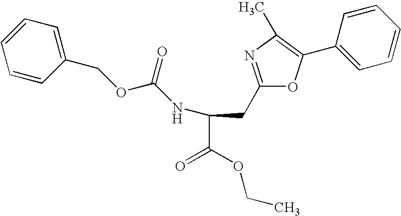

Phosphorous oxychloride (671 μL, 7 mmol) was added to a solution of the amide of preparation 12 (1.025 g, 2 mmol) in toluene (15 mL) and the reaction mixture stirred at 100° C. for 90 minutes. The reaction mixture was poured onto ice (20 mL), extracted into dichloromethane (2×50 mL), dried over magnesium sulphate and concentrated in vacuo. The crude product was purified by column chromatography eluting with pentane:ethyl acetate (80:20 to 66:33) to give the title product, 598 mg; ¹HNMR (CDCl₃, 400 MHz): 1.20 (m, 3H), 2.37 (s, 3H), 3.21-3.41 (brm, 2H), 4.11 (m, 2H), 4.78 (brs, 1H), 5.14 (s, 2H), 5.92 (s, 1H), 7.38 (m, 8H), 7.50 (m, 2H); MS ES+ m/z 431 [MNa]⁺.

The following compounds were made by a similar method to that described in preparation 27, using the appropriate heterocycle precursor.

Preparation 28

(2S)-2-Benzyloxycarbonylamino-3-[5-(4-fluoro-phenyl)-oxazol-2-yl]-propionic Acid Ethyl Ester

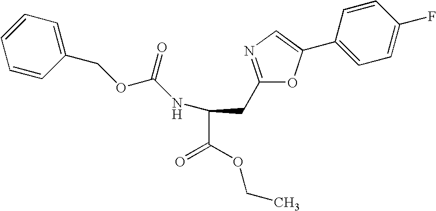

¹HNMR (CDCl₃, 400 MHz): 1.21 (t, 3H), 3.25-3.42 (brm, 2H), 4.19 (m, 2H), 4.80 (brs, 1H), 5.11 (s, 2H), 5.96 (brs, 1H), 7.08 (m, 3H), 7.31 (m, 5H), 7.54 (m, 2H); MS ES+ m/z 435 [MNa]⁺.

Preparation 29

(2S)-2-Benzyloxycarbonylamino-3-(5-phenyl-oxazol-2-yl)-propionic Acid Ethyl Ester

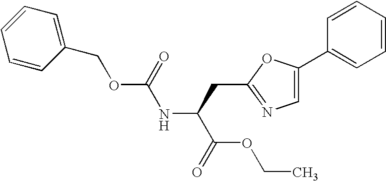

¹HNMR (CDCl₃, 400 MHz): 1.20 (m, 3H), 3.40 (m, 2H), 4.21 (m, 2H), 4.82 (s, 1H), 5.13 (s, 2H), 5.96 (d, 1H), 7.17 (s, 1H), 7.21-7.42 (brm, 10H); MS ES+ m/z 811 [M₂Na]⁺.

Preparation 30

(3S)-3-Benzyloxycarbonylamino-4-(5-phenyl-oxazol-2-yl)-butyric Acid Methyl Ester

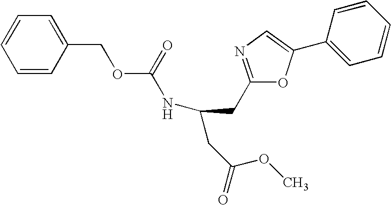

After cooling to 0° C., the reaction mixture was extracted into ethyl acetate (40 mL) and then washed with saturated sodium hydrogencarbonate solution (30 mL); ¹HNMR (CDCl₃, 400 MHz): 2.72 (m, 2H), 3.21 (m, 2H), 3.68 (s, 3H), 4.14 (m, 1H), 5.08 (m, 2H), 5.77 (m, 1H), 7.20 (m, 1H), 7.22-7.41 (m, 10H); MS ES+ m/z 395 [MH]⁺.

Preparation 31

(2S)-2-Benzyloxycarbonylamino-3-[5-(4-chloro-phenyl)-oxazol-2-yl]-propionic Acid Ethyl Ester

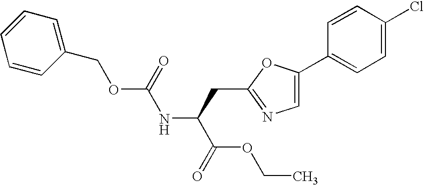

Phosphorous oxychloride (3.6 mL) was added to a solution of the amide of preparation 16 (8.62 g, 19.3 mmol) in toluene (150 mL) and the reaction mixture stirred at 100° C. for 1 hour. The reaction mixture was poured onto ice (20 mL), extracted into dichloromethane (400 mL and 200 mL), dried over magnesium sulphate and concentrated in vacuo. Ether (100 mL) was added, the reaction mixture shaken and concentrated in vacuo. The crude product was purified by column chromatography on silica gel eluting with pentane:ethyl acetate (80:20 to 50:50). The appropriate fractions were purified again by column chromatography on silica gel eluting with pentane:ethyl acetate (80:20 to 75:25 to 66:33 to 50:50) to yield the title product; ¹HNMR (CDCl₃, 400 MHz): 1.24 (t, 3H), 3.34-3.49 (brm, 2H), 4.20 (m, 2H), 4.82 (s, 1H), 5.12 (s, 2H), 5.93 (d, 1H), 7.19 (s, 1H), 7.24 (s, 5H), 7.37 (m, 2H), 7.46 (d, 2H); MS APCI m/z 429[MH]⁺.

Preparation 32

(2S)-2-tert-Butoxycarbonylamino-3-(4-phenyl-oxazol-2-yl)-propionic Acid Benzyl Ester

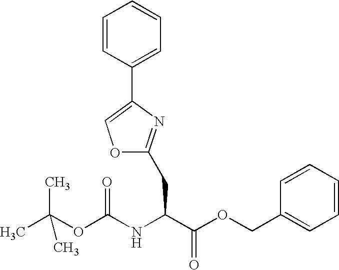

A solution of triphenylphosphine (1.3 g, 4.96 mmol), triethylamine (1.3 mL, 9.34 mmol) and iodine (1.1 g, 4.34 mmol) in tetrahydrofuran (10 mL) was cooled to −78° C. A solution of the amide of preparation 23 (0.96 g, 2.18 mmol) in tetrahydrofuran (7 mL) was added and the reaction mixture stirred at −78° C. for 1 hour. The reaction mixture was then stirred at −20° C. for one hour and then for 1 hour at room temperature. The reaction mixture was diluted with dichloromethane (50 mL), washed with sodium thiosulphate solution (50 mL) and 2M hydrochloric acid (50 mL), dried over magnesium sulphate and concentrated in vacuo. The crude product was purified by column chromatography on silica gel eluting with pentane:ethyl acetate (80:20) to yield the title product, 230 mg; ¹HNMR (CDCl₃, 400 MHz): 1.43 (s, 9H), 3.29 (m, 2H), 4.83 (m, 1H), 5.18 (m, 2H), 5.68 (m, 1H), 7.47 (m, 7H), 7.74 (m, 3H), 7.79 (m, 1H); MS TSP m/z 423 [MH]⁺.

Preparation 33

(2S)-2-Benzyloxycarbonylamino-3-(5-phenyl-[1.3.4]oxadiazol-2-yl)-propionic Acid Ethyl Ester

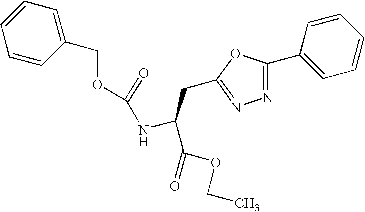

The amide of preparation 15 (650 mg, 1.6 mmol) was dissolved in dichloromethane (10 mL) and the reaction mixture treated with 2-chloro-1,3-dimethyl-4,5-dihydro-3H-imidazol-1-ium tetrafluoroborate (657 mg, 2.4 mmol) and triethylamine (445 μL, 3.2 mmol). The reaction mixture was stirred at room temperature for 18 hours before being concentrated in vacuo. The crude product was purified by column chromatography on silica gel eluting with pentane:ethyl acetate (100:0 to 50:50). The collected product was dissolved in toluene and heated at 80° C. for 8 hours. The reaction mixture was concentrated in vacuo and the residue dissolved in dichloromethane (10 mL) before being washed with 2M hydrochloric acid (5 mL) and saturated sodium hydrogencarbonate solution (5 mL). The reaction mixture was then dried over magnesium sulphate and concentrated in vacuo. The crude product was purified by column chromatography on silica gel eluting with pentane:ethyl acetate (90:10 to 80:20) to yield the title product, 12 mg; ¹HNMR (CDCl₃, 400 MHz):1.25 (t, 3H), 3.49 (m, 2H), 4.22 (m, 2H), 4.87 (m, 1H), 5.16 (m, 2H), 5.87 (m, 1H), 7.32 (m, 5H), 7.51 (m, 3H), 7.99 (m, 2H); MS ES+ m/z 813 [M₂Na]⁺.

Preparation 34

(2S)-2-tert-Butoxycarbonylamino-3-(3-phenyl-[1.2.4]oxadiazol-5-yl)propionic Acid Benzyl Ester

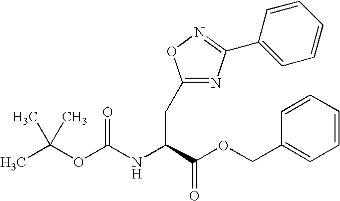

The amide of preparation 19 (815 mg) was heated to 114° C. for 5 hours and the melted solid then allowed to cool to room temperature. The product was triturated with pentane:ether to yield the title product, 693 mg; ¹HNMR (CDCl₃, 400 MHz): 1.44 (s, 9H), 3.48 (dd, 1H), 3.47 (dd, 1H), 4.92 (m, 1H), 5.20 (m, 2H), 5.61 (m, 1H), 7.25 (m, 5H), 7.51 (m, 3H), 8.02 (m, 2H); MS ES+ m/z 446 [MNa]⁺.

Preparation 35

(2S)-2-Benzyloxycarbonylamino-3-(4-phenyl-oxazol-2-yl)-propionic Acid Ethyl Ester

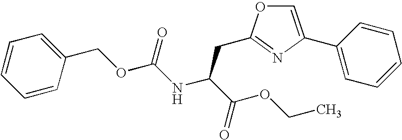

2,6 Di-tert-butyl-pyridine (1.4 mL, 6.1 mmol), 1,2-dibromo-tetrachloroethane (1.98 g, 6.1 mmol) and triphenylphosphine (1.59 g, 6.1 mmol) were added to a solution of the amide of preparation 24 (2.1 g, 5.1 mmol) in dichloromethane (30 mL) and the reaction mixture stirred at room temperature for 30 minutes. 1,8 Diazabicyclo(5.4.0)undec-7-ene (910 μL, 6.1 mmol) was added and the reaction mixture stirred for a further 50 minutes. The reaction mixture was diluted with dichloromethane (50 mL), washed with 2M hydrochloric acid (30 mL), sodium hydrogencarbonate solution (30 mL) and brine (30 mL), dried over magnesium sulphate and concentrated in vacuo. The crude product was purified by column chromatography on silica gel eluting with ethyl acetate:pentane (20:80 to 30:70) to yield the title product, 700 mg; ¹HNMR (CDCl₃, 400 MHz): 1.22 (m, 3H), 3.33 (m, 1H), 3.41 (m, 1H), 3.47 (m, 1H), 4.20 (m, 2H), 4.81 (m, 1H), 5.12 (m, 2H), 7.24-7.42 (m, 8H), 7.66 (m, 2H), 7.82 (m, 1H); MS ES− m/z 393 [M−H]⁻.

The following compounds were made using the preparation described in preparation 35 and the appropriate heterocycle precursor.

Preparation 36

(2S)-2-tert-Butoxycarbonylamino-3-(4-isobutyl-oxazol-2-yl)-propionic Acid Benzyl Ester

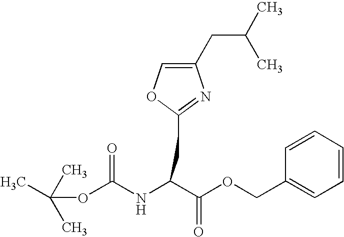

¹HNMR (CDCl₃, 400 MHz): 0.91 (d, 6H), 1.24 (m, 1H), 1.41 (s, 9H), 2.29 (m, 2H), 3.20 (m, 1H), 3.28 (m, 1H), 4.77 (m, 1H), 5.15 (m, 2H), 5.59 (m, 1H), 7.32 (m, 5H); MS ES+ m/z 403 [MH]⁺.

Preparation 37

(2S)-2-tert-Butoxycarbonylamino-3-(4-ethyl-oxazol-2-yl)-propionic Acid Benzyl Ester

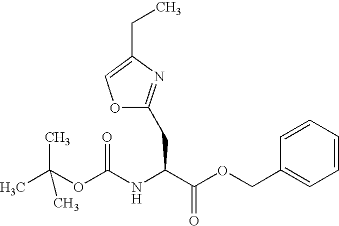

¹HNMR (CDCl₃, 400 MHz): 1.17 (t, 3H), 1.40 (s, 9H), 2.46 (m, 2H), 3.20 (m, 1H), 3.31 (m, 1H), 4.76 (m, 1H), 5.16 (m, 2H), 5.59 (m, 1H), 7.22 (m, 1H), 7.33 (m, 5H); MS ES+ m/z 375 [MH]⁺.

Preparation 38

3,6-Diethoxy-(2R)-2-isopropyl-(5S)-5-(2-phenyl-oxazol-5-ylmethyl)-2,5-dihydro-pyrazine

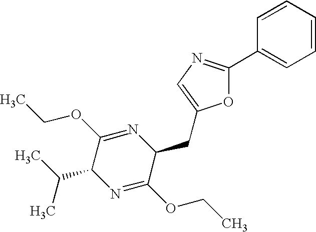

n-Butyllithium (3.4 mL, 5.46 mmol) was added dropwise to a solution of the pyrazine of preparation 3 (1.05 g, 4.97 mmol) in tetrahydrofuran (10 mL) at −60° C. and the reaction mixture stirred for 1 hour. A solution of 5-bromomethyl-2-phenyl-oxazole (J. Chem. Soc. Perkin Trans. 1 1984(2) 255-260 compound 19) (1.3 g, 5.46 mmol) in tetrahydrofuran (10 mL) was added dropwise to the reaction mixture at −60° C. and the reaction warmed for 18 hours at room temperature. The reaction mixture was quenched with ammonium chloride solution (30 mL), and extracted into ethyl acetate (2×50 mL). The organics were combined, washed with brine (50 mL), dried over magnesium sulphate and concentrated in vacuo. The crude product was purified by column chromatography on silica gel eluting with pentane:ethyl acetate (95:5 to 90:10) to yield the title product, 1.33 g; ¹HNMR (DMSOD₆, 400 MHz): 0.60 (m, 3H), 0.91 (m, 3H), 1.20 (m, 3H), 1.26 (m, 3H), 2.15 (m, 1H), 2.83 (dd, 1H), 2.98 (dd, 1H), 3.70 (m, 1H), 3.97-4.16 (brm, 4H), 4.24 (m, 1H), 7.51 (m, 3H), 7.79 (s, 1H), 7.91 (m, 2H); MS APCI m/z 370 [MH]⁺.

Preparation 39

(2R)-2-Isopropyl-3,6-dimethoxy-(5S)-5-(5-phenyl-[1,2,4]oxadiazol-3-ylmethyl)-2,5-dihydro-pyrazine

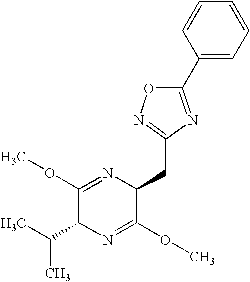

This compound was prepared by the method described in preparation 38 above using 2-isopropyl-3,6-dimethoxy-2,5-dihydro-pyrazine and 3-chloromethyl-5-phenyl-[1,2,4]oxadiazole as the starting materials; ¹HNMR (CDCl₃, 400 MHz): 0.71 (m, 3H), 1.04 (m, 3H), 2.22 (m, 1H), 3.16 (m, 1H), 3.37 (m, 1H), 3.61 (s, 6H), 3.85 (m, 1H), 4.46 (m, 1H), 7.57 (m, 3H), 8.09 (m, 2H); MS ES+ m/z 707 [M₂Na]⁺.

Preparation 40

3,6-Diethoxy-(2R)-2-isopropyl-(5S)-5-(5-phenyl-oxazol-4-ylmethyl)-2,5-dihydro-pyrazine

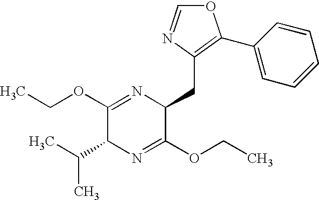

This compound was prepared by the method described in preparation 38 and the pyrazine of preparation 3 and 4-bromomethyl-5-phenyl-oxazole as the starting materials.

¹HNMR (CDCl₃, 400 MHz): 0.67 (d, 3H), 1.04 (d, 3H), 1.11 (m, 3H), 1.26 (m, 3H), 2.24 (m, 1H), 3.15 (dd, 1H), 3.37 (dd, 1H), 3.74 (m, 2H), 3.92 (m, 1H), 4.06 (m, 1H), 4.23 (m, 1H), 4.42 (m, 1H), 7.35 (m, 1H), 7.40 (m, 2H), 7.69 (m, 2H), 7.81 (m, 1H); MS APCI m/z 370 [MH]⁺.

Preparation 41

(2S)-2-tert-Butoxycarbonylamino-3-(4-phenyl-imidazol-1-yl)-propionic Acid

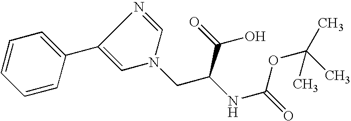

4-Phenylimidazole (265 mg, 1.84 mmol) was added to a solution of (2-oxo-oxetan-3-yl)-carbamic acid tert-butyl ester (WO 9007111, page 37, step 4) (370 mg, 1.84 mmol) in acetonitrile (10 mL) and the reaction mixture stirred at room temperature for 5 days. 68 mg of a white crystalline solid was collected by filtration and trituration of the reaction mixture followed by filtration yielded an additional 100 mg; ¹HNMR (CDCl₃+TFA, 400 MHz): 1.50 (s, 9H), 4.46 (m, 1H), 4.68 (dd, 1H), 4.80 (dd, 1H), 5.82 (m, 1H), 7.21 (m, 1H), 7.32 (m, 1H), 7.39 (m, 2H), 7.61 (m, 2H), 8.64 (s, 1H); MS APCI m/z 332 [MH]⁺.

Preparation 42

(2S)-2-tert-Butoxycarbonylamino-3-(4-iodo-pyrazol-1-yl)-propionic Acid Benzyl Ester

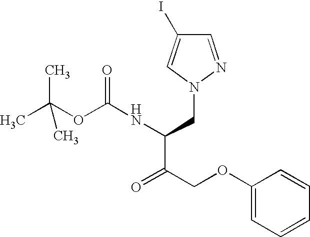

4-Iodopyrazole (0.84 g, 4 mmol) and boron trifluoride diethyl ether complex (1 drop) were added to a solution of the product of preparation 2 (0.8 g, 3 mmol) in dichloromethane (10 mL) and the reaction mixture stirred at room temperature for 6 hours. The reaction mixture was concentrated in vacuo and purified by column chromatography on silica gel eluting with pentane:ethyl acetate 89:11 to 80:20; MS ES+ m/z 494 [MNa]⁺.

Preparation 43

(2S)-2-tert-Butoxycarbonylamino-3-(4-phenyl-pyrazol-1-yl)-propionic Acid Benzyl Ester

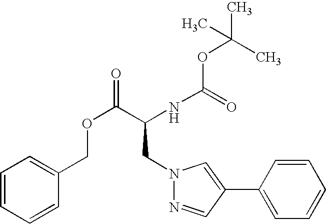

Tris(dibenzylideneacetone)dipalladium(0) (20 mg, 0.22 mmol) was dissolved in N,N-dimethylformamide (5 mL) and the pyrazole of preparation 42 (227 mg, 0.48 mmol) added. The reaction mixture was heated to 60° C. and tri-n-butylphenyl tin (212 mg, 0.58 mmol) and copper iodide (20 mg) added. The reaction mixture was stirred at 60° C. for 90 minutes before being concentrated in vacuo and diluted with ether (20 mL). The reaction mixture was washed with 10% potassium fluoride solution, dried over magnesium sulphate and purified by column chromatography on silica gel eluting with pentane:ethyl acetate (89:11 to 80:20) to yield the title product, 53 mg; ¹HNMR (CDCl₃, 400 MHz): 1.44 (s, 9H), 4.58 (m, 1H), 4.67 (m, 2H), 5.17 (m, 1H), 5.21 (m, 2H), 5.64 (m, 1H), 7.18-7.43 (m, 10H), 7.76 (m, 2H); MS ES+ m/z 444 [MNa]⁺.

Preparation 44

(2S)-2-Benzyloxycarbonylamino-3-(5-phenyl-oxazol-2-yl)-propionic Acid

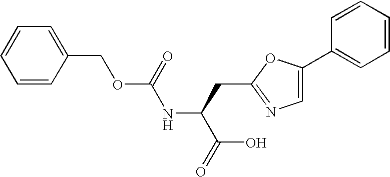

The ester of preparation 29 (1.5 g, 3.8 mmol) was added to a solution of 1 M sodium hydroxide (11.4 mL) in methanol (40 mL) and the reaction stirred at room temperature for 18 hours. The reaction mixture was then concentrated in vacuo and suspended in 2M hydrochloric acid (30 mL). The product was extracted into ethyl acetate (2×50 mL) dried over magnesium sulphate and concentrated in vacuo to yield the title product, 1.4 g; ¹HNMR (CDCl₃, 400 MHz): 3.60 (m, 2H), 4.82 (m, 1H), 5.13 (m, 2H), 5.96 (m, 1H), 7.30-7.44 (m, 9H), 7.57 (m, 2H); MS ES+ m/z 367 [MH]⁺.

Preparation 45

(2S)-2-Amino-3-(2-phenyl-oxazol-5-yl)-propionic Acid Ethyl Ester

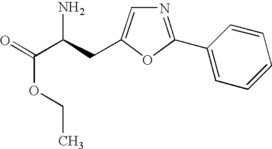

0.25M Hydrochloric acid (30 mL) was added to a solution of the pyrazine of preparation 38 (1.29 g, 3.5 mmol) in tetrahydrofuran (30 mL) and the reaction mixture stirred at room temperature for 5 hours. The reaction mixture was concentrated in vacuo, basified with sodium hydrogencarbonate solution (60 mL) and extracted into ethyl acetate (3×30 mL). The organics were combined, washed with brine, dried over magnesium sulphate and concentrated in vacuo. The crude product was purified by column chromatography on silica gel eluting with pentane:ethyl acetate (50:50 to 40:60 to 30:70) to yield the title product, 567 mg; ¹HNMR (CDCl₃, 400 MHz): 1.24 (t, 3H), 3.01 (dd, 1H), 3.19 (dd, 1H) 4.02 (m, 1H), 4.20 (m, 2H), 7.43 (m, 3H), 7.38 (m, 1H), 8.01 (m, 2H); MS ES+ m/z 283 [MNa]⁺.

Preparation 46

(2S)-2-Amino-3-(5-phenyl-[1,2,4]oxadiazol-3-yl)-propionic Acid Methyl Ester

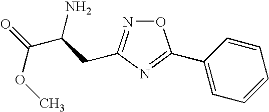

0.25M Hydrochloric acid (15 mL) was added to a solution of the pyrazine of preparation 39 (417 mg, 1.22 mmol) in tetrahydrofuran (3 mL) and the reaction stood at room temperature for 18 hours. The reaction mixture was extracted with ethyl acetate (2×10 mL) and the organic extracts washed with saturated sodium bicarbonate solution (10 mL), water (10 mL) and brine (10 mL). The solution was dried over magnesium sulphate and concentrated in vacuo. The crude product was purified by column chromatography on silica gel eluting with dichloromethane:methanol (98:2) to yield the title product, 250 mg; ¹HNMR (CDCl₃, 400 MHz: 3.17 (dd, 1H), 3.32 (dd, 1H), 3.78 (s, 3H), 4.07 (m, 1H), 7.54 (m, 3H), 8.12 (m, 2H); MS ES+m/z 248 [MH]⁺.

Preparation 47

(2S)-2-Amino-3-(5-phenyl-oxazol-4-yl)-propionic Acid Ethyl Ester

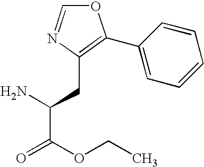

This compound was prepared by the method described in preparation 46 from the pyrazine of preparation 40; ¹HNMR (CDCl₃, 400 MHz): 1.21 (t, 3H), 2.20 (m, 2H), 3.18 (dd, 1H), 3.34 (dd, 1H), 4.06 (m, 1H), 4.13 (m, 2H), 7.36 (m, 1H), 7.45 (m, 2H), 7.65 (m, 2H), 7.88 (m, 1H); MS APCI m/z 261 [MH]⁺.

Preparation 48

(2S)-2-tert-Butoxycarbonylamino-3-(4-phenyl-imidazol-1-yl)-propionic Acid Ethyl Ester

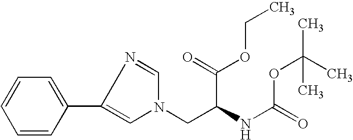

Ethanol (1 mL) was added to a solution of the carboxylic acid of preparation 41 (135 mg, 0.41 mmol), 1-(3-dimethylaminopropyl)-3-ethylcarbodiimide hydrochloride (0.86 mg, 1.64 mmol), 1-hydroxybenzotriazole hydrate (0.61 mg, 0.44 mmol) and 4-Methylmorpholine (165 mg, 1.64 mmol) in dichloromethane (10 mL) and the reaction mixture stirred at room temperature for 18 hours. The reaction mixture was poured into water (50 mL) and extracted with dichloromethane (3×50 mL). The organics were combined, washed with brine (50 mL), dried over magnesium sulphate and concentrated in vacuo to yield the title product, 161 mg; ¹HNMR (CDCl₃, 400 MHz): 1.26 (m, 3H), 1.44 (s, 9H), 4.26 (m, 2H), 4.70 (m, 2H), 4.81 (m, 1H), 5.36 (m, 1H), 7.18 (m, 1H), 7.28 (m, 1H), 7.39 (m, 2H), 7.77 (m, 2H), 7.84 (m, 1H); MS ES+ m/z 382 [MNa]⁺.

Preparation 49

(2S)-2-Benzyloxycarbonylamino-3-(5-phenyl-oxazol-2-yl)-propionic Acid Tert-butyl Ester

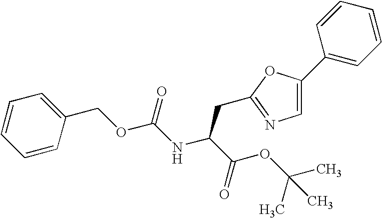

The carboxylic acid of preparation 44 (1.4 g, 3.8 mmol) was added to a solution of N,N′-dicyclohexylcarbodiimide (0.87 g, 4.2 mmol), dimethyl-pyridin-4-yl-amine (50 mg, 0.41 mmol) and tert-butanol (1.1 g, 14.9 mmol) in dichloromethane (20 mL) and the reaction mixture stirred at room temperature for 18 hours. The reaction mixture was filtered to remove insolubles and concentrated in vacuo. The crude product was purified by column chromatography on silica gel eluting with pentane:ethyl acetate (87.5:12.5 to 80:20 to 50:50) to yield the title product, 980 mg; ¹HNMR (CDCl₃, 400 MHz): 1.41 (s, 9H), 3.28 (dd, 1H), 3.41 (dd, 1H), 4.74 (m, 1H), 5.18 (m, 2H), 5.88 (m, 1H) 7.22 (m, 2H), 7.39 (m, 7H), 7.62 (m, 2H); MS ES− m/z 421 [M−H]⁻.

Preparation 50

(2S)-2-Amino-3-[5-(4-fluoro-phenyl)-oxazol-2-yl]-propionic Acid Ethyl Ester

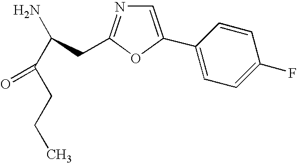

A solution of hydrogen bromide in acetic acid (1.1 mL) was added to a solution of the protected amine of preparation 28 (547 mg, 1.23 mmol) in acetic acid (1.1 mL) and the reaction stirred at room temperature for 1 hour. The reaction mixture was concentrated in vacuo, diluted with ethyl acetate (20 mL) and neutralised with sodium hydrogencarbonate solution. The organic layer was separated and dried over magnesium sulphate and concentrated in vacuo. The crude product was purified by column chromatography on silica gel, eluting with ethyl acetate:dichloromethane:methanol:ammonium hydroxide (100:0:0:0 to 0:90:10:1) to yield the title product, 180 mg; ¹HNMR (CDCl₃, 400 MHz): 1.22 (t, 3H), 3.05-3.36 (brm, 2H), 3.95 (m, 1H), 4.19 (m, 2H), 7.07 (m, 2H) 7.12 (m, 1H), 7.56 (m, 2H); MS ES+ m/z 301 [MNa]⁺.

The following compounds were made by a method similar to that described in preparation 50 using the appropriate CBz protected amine.

Preparation 51

(2S)-2-Amino-3-(4-methyl-5-phenyl-oxazol-2-yl)-propionic Acid Ethyl Ester

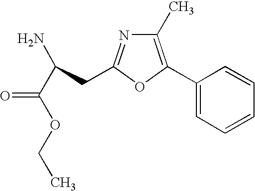

¹HNMR (CDCl₃, 400 MHz): 1.22 (t, 3H), 2.37 (2, 3H), 3.13 (m, 1H), 3.22 (m, 1H), 3.97 (m, 1H), 4.18 (m, 2H), 7.24 (m, 1H), 7.35 (m, 2H), 7.52 (m, 2H); MS ES+ m/z 297 [MNa]⁺.

Preparation 52

(2S)-2-Amino-3-(5-phenyl-oxazol-2-yl)-propionic Acid Ethyl Ester Hydrochloride

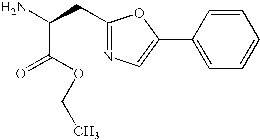

This compound was isolated after trituration with 2M hydrochloric acid in ether instead.

¹HNMR (CDCl₃, 400 MHz): 1.09 (t, 3H), 3.22 (s, 2H), 4.13 (m, 2H), 4.32 (m, 3H), 7.21-7.74 (brm, 6H); MS ES+m/z 261 [MH]⁺.

Preparation 53

(2S)-2-Amino-3-[5-(4-chloro-phenyl)-oxazol-2-yl]-propionic Acid Ethyl Ester Hydrochloride

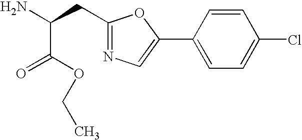

This compound was not purified by column chromatography but was instead dissolved in dichloromethane (40 mL), washed with hydrochloric acid in ether and concentrated in vacuo; [α]=+31.82; ¹HNMR (CDCl₃, 400 MHz): 1.24 (t, 3H), 3.52 (d, 2H), 4.31 (m, 2H), 4.60 (t, 1H), 7.47 (d, 3H), 7.68 (d, 2H); MS APCI m/z 295 [MH]⁺.

Preparation 54

(3S)-3-Amino-4-(5-Phenyl-oxazol-2-yl)-butyric Acid Methyl Ester

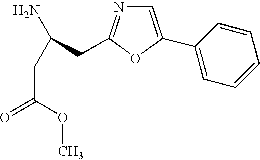

¹HNMR (CDCl₃, 400 MHz): 2.64 (m, 2H), 3.04 (m, 2H), 3.71 (s, 3H), 4.36 (m, 1H), 7.23 (m, 1H), 7.31 (m, 1H), 7.39 (m, 2H), 7.58 (m, 2H); MS ES+ m/z 261 [MH]⁺.

Preparation 55

(2S)-2-Amino-3-(5-phenyl-oxazol-2-yl)-propionic Acid Hydrobromide

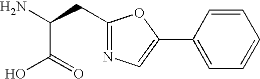

Hydrogen bromide in acetic acid (1 mL) was added to a solution of the CBz protected amine of preparation 44 (435 mg, 1.19 mmol) in acetic acid (3 mL) and the reaction mixture stirred at room temperature for 18 hours. Diethyl ether (5 mL) was added to the reaction mixture before being sonicated, filtered and dried under high vacuum to yield the title product, 410 mg; ¹HNMR (DMSOD₆, 400 MHz): 3.41 (m, 2H), 4.44 (m, 1H), 7.36 (m, 1H), 7.44 (m, 2H), 7.59 (m, 1H), 7.71 (m, 2H), 8.40 (m, 3H); MS ES+ m/z 233 [MH]⁺.

Preparation 56

(2S)-2-Amino-3-(5-phenyl-oxazol-2-yl)-propionic Acid Tert Butyl Ester

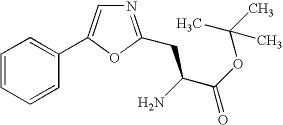

A solution of the protected amine of preparation 49 (0.98 g, 2.3 mmol) in tetrahydrofuran (5 mL) was treated was 10% Pd/C (100 mg) and the reaction mixture stirred at room temperature and 15 psi for 48 hours. The reaction mixture was filtered through Arbocel®, washing through with dichloromethane, and concentrated in vacuo. The crude product was purified by column chromatography on silica gel eluting with pentane:ethyl acetate 50:50 to dichloromethane:methanol 95:5 to yield the title product, 460 mg; ¹HNMR (CDCl₃, 400 MHz): 1.43 (s, 9H), 3.19 (dd, 1H), 3.37 (dd, 1H), 3.99 (m, 1H), 7.22 (m, 2H), 7.26 (m, 1H), 7.37 (m, 1H), 7.73 (m, 2H); MS ES+ m/z 289 [MH]⁺.

Preparation 57

(2S)-2-Amino-3-(5-phenyl-[1,3,4]oxadiazol-2-yl)-propionic Acid Ethyl Ester

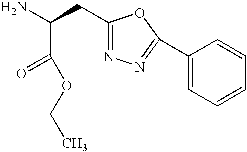

The protected amine of preparation 33 (200 mg, 5 mmol) was added to a solution of 10% Pd/C (20 mg) in ethanol (7 mL) and the reaction mixture stirred at room temperature at 50 psi under hydrogen for 18 hours. The reaction mixture was filtered through Arbocel® and concentrated in vacuo. The product was redissolved in ethanol (5 mL), treated with more Pd/C (50 mg) and returned to the above conditions for a further 18 hours. The reaction mixture was filtered through Arbocel® and concentrated in vacuo. The crude product was purified by column chromatography on silica gel eluting with (95:5:0.5) dichloromethane:methanol:ammonium hydroxide to yield the title product, 50 mg; ¹HNMR (CDCl₃, 400 MHz): 1.23 (t, 3H), 1.85 (m, 2H), 3.27 (dd, 1H), 3.38 (dd, 1H), 3.41 (m, 1H), 4.07 (m, 1H), 4.24 (m, 2H), 7.52 (m, 3H), 8.04 (m, 2H); MS ES+ m/z 523 [M₂H]⁺.

Preparation 58

(2S)-2-Amino-3-(4-phenyl-oxazol-2-yl)-propionic Acid Ethyl Ester

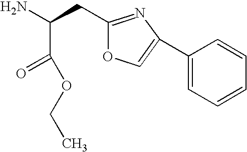

10% Pd/C (100 mg) was added to a solution of the protected amine of preparation 35 (700 mg, 178 mmol) in ethanol (10 mL) and the reaction mixture stirred at room temperature and 50 psi under hydrogen for 18 hours. The reaction mixture was filtered through Arbocel®, washed with methanol and concentrated in vacuo. The crude product was purified by column chromatography on silica gel eluting with dichloromethane:methanol:acetic acid 95:5:0.5 to yield the title product, 330 mg; ¹HNMR (CDCl₃, 400 MHz): 1.04 (t, 3H), 1.82 (m, 2H), 3.16 (m, 1H), 3.27 (m, 1H), 3.98 (m, 1H), 4.19 (m, 2H), 7.23 (m, 1H), 7.39 (m, 2H), 7.71 (m, 2H), 7.83 (m, 1H).

Preparation 59

(2S)-2-Amino-3-(4-phenyl-oxazol-2-yl)propionic Acid Benzyl Ester

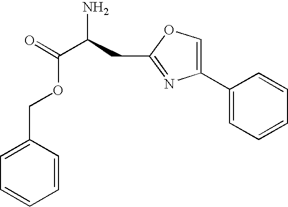

Trifluoroacetic acid (1 mL) was added to a solution of the protected amine of preparation 32 (220 mg, 0.5 mmol) in dichloromethane (5 mL) and the reaction mixture stirred for 1 hour at room temperature. The reaction mixture was diluted with ethyl acetate (25 mL), washed with saturated aqueous sodium hydrogencarbonate solution (10 mL), dried over magnesium sulphate and concentrated in vacuo, to yield the title product; MS ES+ m/z 323[MH]⁺.

Preparation 60

(2S)-2-Amino-3-(4-phenyl-pyrazol-1-yl)-propionic Acid Benzyl Ester

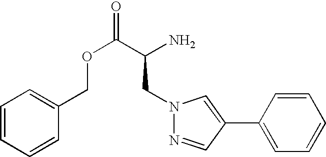

The protected amine of preparation 43 (185 mg, 0.44 mol) was added to a solution of trifluoroacetic acid (2 mL) in dichloromethane (4 mL) and the reaction mixture stirred at room temperature for 2 hours. The reaction mixture was concentrated in vacuo, diluted with ethyl acetate (20 mL) and washed with sodium hydrogencarbonate solution (10 mL). The organic layer was dried over magnesium sulphate and concentrated in vacuo to yield the title product, 142 mg; MS ES+ m/z 344 [MNa]⁺.

Preparation 61

(2S)-2-Amino-3-(3-phenyl-[1.2.4]oxadiazol-5-yl)-propionic Acid Benzyl Ester Trifluoroacetate

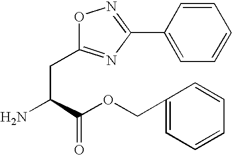

The protected amine of preparation 34 (617 mg) was added to a solution of trifluoroacetic acid (5 mL) in dichloromethane (5 mL) and the reaction mixture stirred at room temperature for 1 hour. The reaction mixture was concentrated in vacuo and azeotroped twice with toluene and then with ethyl acetate and ether to yield the title product, 635 mg; ¹HNMR (CD₃OD, 400 MHz): 3.71 (m, 2H), 4.76 (m, 1H), 5.25 (dd, 1H), 5.36 (dd, 1H), 7.22 (m, 3H), 7.34 (m, 2H), 7.52 (m, 3H), 8.02 (m, 2H); MS ES+ m/z 346 [MNa]⁺.

Preparation 62

(2S)-2-Amino-3-(4-isobutyl-oxazol-2-yl)-propionic Acid Benzyl Ester

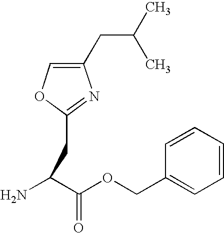

The protected amine of preparation 36 (0.92 g, 2.3 mmol) was dissolved in dichloromethane (20 mL) and the reaction mixture treated with trifluoroacetic acid (5 mL). The yellow solution was stirred for 4 hours at room temperature. The reaction mixture was concentrated in vacuo and the residue diluted with ethyl acetate (20 mL). The reaction mixture was then washed with sodium hydrogencarbonate solution (20 mL), dried over magnesium sulphate and concentrated in vacuo to yield the title product; MS ES+ m/z 325 [MNa]⁺.

Preparation 63

(2S)-2-Amino-3-(4-ethyl-oxazol-2-yl)-propionic Acid Benzyl Ester

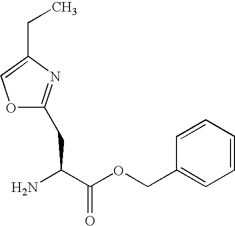

The title compound was prepared from the amine from preparation 37 by the method described in preparation 62; MS ES+ m/z 275 [MH]⁺.

Preparation 64

(2S)-2-Amino-3-(4-phenyl-imidazol-1-yl)-propionic Acid Ethyl Ester

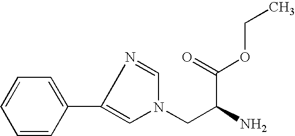

The protected amine of preparation 48 (108 mg, 0.3 mmol) was added to 4M hydrochloric acid in dioxan (5 mL) and the reaction mixture stirred for 18 hours at room temperature. The reaction mixture was concentrated in vacuo and the residue partitioned between dichloromethane (50 mL) and potassium carbonate solution (50 mL). The aqueous phase was extracted with more dichloromethane (50 mL) and the organics were combined, dried over magnesium sulphate and concentrated in vacuo to yield the title product, 79 mg; ¹HNMR (CDCl₃, 400 MHz): 1.26 (m, 3H), 1.43 (m, 1H), 1.97 (m, 2H), 3.77 (m, 1H) 4.22 (m, 3H), 7.40 (m, 2H), 7.58 (m, 1H), 7.77 (m, 2H).

Preparation 65

(2S)-2-tert-butoxycarbonylamino-3-(5-phenyl-oxazol-2-yl)-propionic Acid

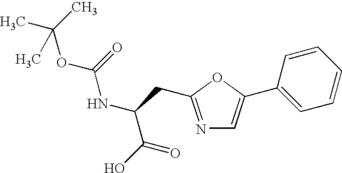

Di-tert-butyl-dicarbonate (343 mg, 1.25 mmol) was added to a solution of the amine of preparation 55 (410 mg, 1.31 mmol) and triethylamine (530 mg, 5.24 mmol) in dichloromethane (5 mL) and the reaction mixture stirred at room temperature for 18 hours. The reaction mixture was concentrated in vacuo and the product purified by column chromatography on silica gel eluting with dichloromethane:methanol (95:5 to 90:10) to yield the title product, 240 mg; ¹HNMR (DMSOD₆, 400 MHz): 1.27 (s, 9H), 3.35 (m, 2H), 4.32 (m, 1H), 7.37 (m, 1H), 7.41 (m, 2H), 7.51 (m, 1H), 7.64 (m, 2H); MS ES+ m/z 333 [MH]⁺.

Preparation 66

(2S)-2-Amino-3-(5-phenyl-oxazol-2-yl)-propionic Acid Propyl Ester

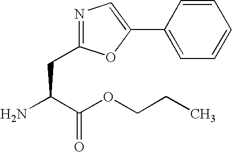

1-Propanol (30 mL) stirring at 0° C. was treated dropwise with acetyl chloride (8 mL) over ten minutes and the reaction mixture warmed to room temperature and stirred for 1 hour. The ester of preparation 52 (600 mg, 2.3 mmol) was added and the reaction mixture heated to 40° C. for 3 hours and then to 70° C. for 18 hours. The reaction mixture was concentrated in vacuo to yield the title product; ¹HNMR (CDCl₃, 400 MHz): 0.80 (t, 3H), 1.56 (m, 2H), 3.41 (m, 2H), 4.06 (m, 2H), 4.57 (s, 1H), 7.16-7.79 (brm, 6H), 8.76 (s, 2H) MS ES+ m/z 275 [MH]⁺.

The following compounds were prepared using a method similar to that described in preparation 66.

Preparation 67

(2S)-2-Amino-3-(5-phenyl-oxazol-2-yl)propionic Acid Isobutyl Ester Hydrochloride

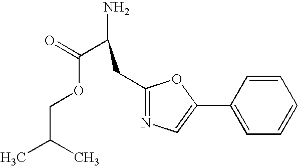

The title compound was prepared from the ester of preparation 52 and isobutanol. After heating at 70° C. for 18 hours the crude product was azeotroped with toluene and concentrated in vacuo; ¹HNMR (DMSOD₆, 400 MHz): 0.91 (m, 6H), 1.82 (m, 1H), 3.46 (m, 2H), 3.91 (m, 2H), 4.58 (m, 1H), 7.33 (m, 1H), 7.42 (m, 2H), 7.62 (m, 1H), 7.66 (m, 2H), 8.77 (m, 2H); MS ES+ m/z 289 [MH]⁺.

Preparation 68

(2S)-2-Amino-3-(5-phenyl-oxazol-2-yl)-propionic Acid Isopropyl Ester Hydrochloride

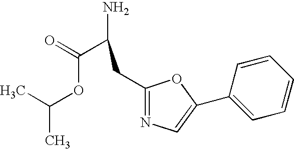

The title compound was prepared from the ester of preparation 52 and isopropanol. After heating at 70° C. for 48 hours the crude product was azeotroped with toluene and concentrated in vacuo; [α]=+26.03; ¹HNMR (DMSOD₆, 400 MHz): 1.02 (dd, 3H), 1.13 (dd, 3H), 3.44 (m, 2H), 4.45 (s, 1H), 4.95 (m, 1H), 7.16 (m, 1H), 7.21 (m, 1H), 7.41 (m, 1H), 7.45 (m, 2H), 7.62 (m, 1H), 8.82 (s, 2H); MS ES+ m/z 275 [MH]⁺.

Preparation 69

(2S)-2-Amino-3-(5-phenyl-oxazol-2-yl)propionic Acid Butyl Ester Hydrochloride

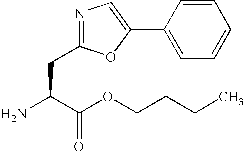

The title compound was prepared from the ester of preparation 52 and n-Butanol. [α]=+25.21; ¹HNMR (CDCl₃, 400 MHz): 0.77 (t, 3H), 1.18 (m, 2H), 1.45 (m, 2H), 3.47 (m, 2H), 4.08 (m, 2H), 4.54 (m, 1H), 7.36 (m, 1H), 7.46 (m, 2H), 7.61 (m, 1H), 7.72 (m, 2H), 8.62 (m, 2H); MS ES+ m/z 289 [MH]⁺.

Preparation 70

(2S)-2-Amino-3-(5-phenyl-oxazol-2-yl)-propionic Acid Cyclopentyl Ester Hydrochloride

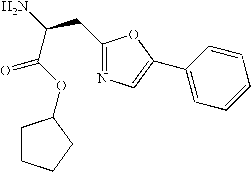

The title compound was prepared from cyclopentanol and the ester of preparation 52. After heating to 70° C. for 72 hours, the crude product was triturated with ether and concentrated in vacuo; ¹HNMR (CDCl₃, 400 MHz): 1.38-1.83 (m, 8H), 4.48 (m, 1H), 5.13 (m, 1H), 7.37 (m, 2H), 7.44 (m, 2H), 7.69 (m, 2H); MS ES+ m/z 301 [MH]⁺.

Preparation 71

(2S)-2-Amino-3-(5-Phenyl-oxazol-2-yl)-propionic Acid 1-ethyl-propyl Ester Hydrochloride

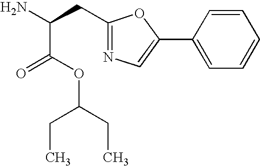

The title compound was prepared from pentan-3-ol and the ester of preparation 52. After heating at 70° C. for 72 hours, the crude product was triturated with ether and concentrated in vacuo; ¹HNMR (DMSOD₆, 400 MHz): 0.80 (s, 6H), 1.37-1.61 (m, 4H), 4.44 (m, 1H), 4.59 (m, 1H), 4.73 (m, 1H), 7.37 (m, 1H), 7.46 (m, 2H), 7.62 (m, 1H), 7.68 (m, 2H), 8.61 (m, 2H); MS ES+ m/z 303 [MH]⁺.

Preparation 72

(3S)-3-Amino-4-(5-phenyl-oxazol-2-yl)-butyric Acid Ethyl Ester Hydrochloride

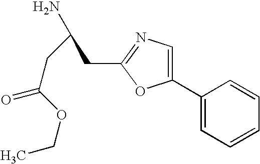

The ester of preparation 54 (720 mg, 2.6 mmol) was added to a saturated solution of hydrochloric acid in ethanol (30 mL) and the reaction mixture stirred at 70° C. for 1 hour. The reaction mixture was concentrated in vacuo, triturated with ether and ethyl acetate and filtered to yield the title product, 614 mg; ¹HNMR (DMSOD₆, 400 MHz): 1.17 (t, 3H), 3.84 (m, 2H), 3.22 (dd, 1H), 3.29 (dd, 1H), 3.92 (m, 1H), 4.09 (m, 2H), 7.36 (m, 1H), 7.42 (m, 2H) 7.60 (m, 1H), 7.64 (m, 2H), 8.45 (m, 2H); MS ES+ m/z 275 [MNa]⁺.

Preparation 73

(2S)-2-Amino-3-(5-phenyl-oxazol-2-yl)-propionic Acid 2-butoxy-ethyl Ester Hydrochloride

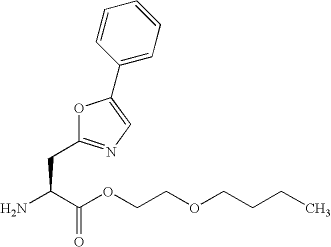

Hydrogen bromide was bubbled through a solution of the ester of preparation 52 (430 mg, 1.26 mmol) in n-butoxyethanol (10 mL) and the reaction mixture stirred at 70° C. for 72 hours. The reaction mixture was concentrated in vacuo and partitioned between ethyl acetate and aqueous sodium bicarbonate solution. The organic layer was dried over magnesium sulphate and concentrated in vacuo. The crude product was purified by column chromatography on silica gel eluting with dichloromethane: methanol 95:5 to 90:100 then triturated with hydrochloric acid in ether to yield the title product, 211 mg; ¹HNMR (CDCl₃, 400 MHz): 0.84 (t, 3H), 1.24 (m, 2H), 1.45 (m, 2H), 3.56 (m, 3H), 3.76 (dd, 1H), 3.92 (dd, 1H), 4.32 (m, 2H), 4.81 (m, 1H), 7.29-7.42 (m, 4H), 7.59 (m, 2H), 9.29 (m, 2H); MS ES+ m/z 355 [MNa]⁺.

Preparation 74

(2S)-2-tert-Butoxycarbonylamino-3-(5-phenyl-oxazol-2-yl)-propionic Acid Benzyl Ester

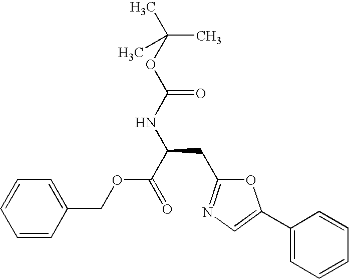

The carboxylic acid of preparation 65 (240 mg, 0.72 mmol) was added to a solution of caesium carbonate (282 mg, 0.87 mmol) in N,N-dimethylformamide (4 mL) and the reaction mixture stirred for 1 hour. The reaction mixture was treated with benzyl bromide (107 mg, 0.87 mmol) and stirred at room temperature for 48 hours. The reaction mixture was partitioned between ethyl acetate, water (×3) and brine. The organic layer was dried over magnesium sulphate, concentrated in vacuo and azeotroped with dichloromethane (×3) to yield the title product, 225 mg; ¹HNMR (DMSOD₆, 400 MHz): 1.32 (s, 9H), 3.34 (m, 3H), 4.59 (m, 1H), 5.23 (m, 2H), 7.31 (m, 5H), 7.43 (m, 3H), 7.53 (m, 1H), 7.62 (m, 2H); MS ES+ m/z 445[MNa]⁺.

Preparation 75

(2S)-2-Amino-3-(5-phenyl-oxazol-2-yl)-propionic Acid Benzyl Ester

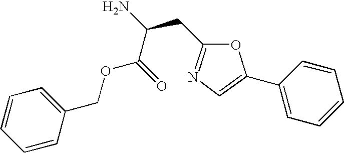

A solution of the protected amine of preparation 74 (220 mg, 0.52 mmol) in dichloromethane (2 mL) was treated with trifluoroacetic acid (1 mL) and the reaction mixture stirred at room temperature for 18 hours. The reaction mixture was concentrated in vacuo and partitioned between ethyl acetate and sodium hydrogencarbonate solution (×2). The organic layer was dried over magnesium sulphate and concentrated in vacuo. The crude product was purified by column chromatography on silica gel eluting with dichloromethane:methanol (95:5) to yield the title product, 127 mg; ¹HNMR (DMSOD₆, 400 MHz): 3.13 (m, 2H), 3.87 (m, 1H), 5.06 (m, 2H), 7.26 (m, 6H), 7.42 (m, 2H), 7.51 (m, 1H), 7.62 (m, 2H); MS ES+ m/z 345 [MNa]⁺.

Preparation 76

(2S)-2-{1-[(1S)-1-Ethoxycarbonyl-2-(4-methyl-5-phenyl-oxazol-2-yl)-ethylcarbamoyl]-cyclopentylmethyl}-4-methoxy-butyric Acid Tert Butyl Ester

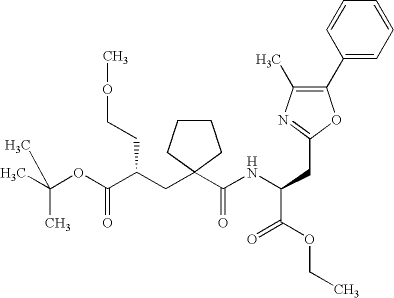

The amine of preparation 51 (1.03 g, 3.7 mmol) was added to a solution of 1-[(2S)-2-(tert-butoxycarbonyl)-4-methoxybutyl]cyclopentanecarboxylic acid (WO 0279143 page 71 preparation 69) (1.3 g, 4 mmol), 1-(3-dimethylaminopropyl)-3-ethylcarbodiimide hydrochloride (0.85 g, 4 mmol), 1-hydroxybenzotriazole hydrate (0.60 g, 4 mmol) and 4-methylmorpholine (1 mL, 9 mmol) in dichloromethane (20 mL) and the reaction mixture stirred at room temperature for 18 hours. The reaction mixture was diluted with ethyl acetate (50 mL), washed with 2M hydrochloric acid (30 mL), dried over magnesium sulphate and concentrated in vacuo. The crude product was purified by column chromatography on silica gel eluting with pentane:ethyl acetate (80:20 to 66:33 to 50:50) to yield the title product, 1.30 g; ¹HNMR (CDCl₃, 400 MHz): 1.21 (s, 3H), 1.38 (s, 9H), 1.56-1.75 (brm, 12H), 2.04 (s, 1H), 2.37 (s, 3H), 3.25 (s, 3H), 3.32 (m, 2H), 3.40 (m, 2H), 4.18 (m, 2H), 4.97 (m, 1H), 7.20 (d, 1H), 7.25 (m, 2H), 7.42 (m, 1H), 7.51 (d, 2H); MS ES+ m/z 579[MNa]⁺.

The following compounds of the general formula shown below were made by the method described in preparation 76 using the appropriate acid and the appropriate amine.

| Prep | R¹ | L-R³ | R⁵ | data |
|---|---|---|---|---|
| 77 | methoxyethyl | 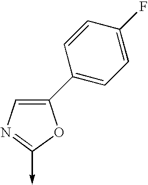 | ethyl | ¹HNMR (CDCl₃, 400MHz): 1.23(m, 3H), 1.40(s, 9H), 1.48-1.77(m, 12H), 2.04(m, 1H) 3.24(s, 3H), 3.32(m, 2H), 3.45(m, 2H), 4.17(m, 2H), 5.04(d, 1H), 7.12(m, 4H), 7.24(s, 1H); MS ES+ m/z 583[MNa]⁺ |
| 78 | propyl | 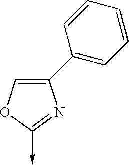 | benzyl | ¹HNMR (CDCl₃, 400MHz): 0.78(m, 3H), 1.34(s, 9H), 1.42-1.81(m, 13H), 3.20-3.48(m, 2H), 5.12(m, 3H), 7.20(m, 5H), 7.26-7.57(m, 6H), 7.77(s, 1H); MS ES+ m/z 611[MNa]⁺ |
| 79¹ | methoxyethyl | 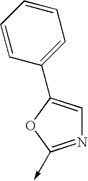 | ethyl | ¹HNMR (CDCl₃, 400MHz): 1.21(m, 3H), 1.39(s, 9H), 1.42-1.81(brm, 13H), 3.22(s, 3H), 3.31(m, 2H), 3.41(m, 2H), 4.19(m, 2H), 5.04(m, 1H), 7.11(d, 1H), 7.20-7.63(brm, 6H), MS ES+m/z 1108 [M₂Na]⁺ |
| 80 | methoxyethyl | 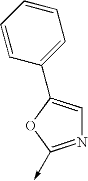 | propyl | ¹HNMR (CDCl₃, 400MHz): 0.84(t, 3H), 1.40(s, 9H), 1.45-1.98(brm, 15H), 3.22(s, 3H), 3.31 (t, 2H), 3.36(d, 1H), 3.47(d, 1H), 4.09(m, 2H), 5.07(m, 1H), 7.10(d, 1H), 7.19-7.64(brm, 6H); MS ES+ m/z 1136 [M₂Na]⁺ |
| 81 | methoxyethyl | 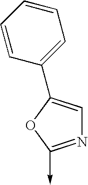 | butyl | ¹HNMR (CDCl₃, 400MHz): 0.82(t, 3H), 1.40(s, 9H), 1.43-2.11(brm, 16H), 2.16(m, 1H), 3.22(s, 3H), 3.31(m, 3H), 3.48(d, 1H), 4.13(m, 2H), 5.04(m, 1H), 7.09(d, 1H), 7.20-7.47(brm, 6H); MS ES+m/z 1164 [M₂Na]⁺ |
| 82 | methoxyethyl | 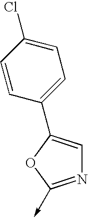 | ethyl | ¹HNMR (CDCl₃, 400MHz): 1.20(s, 3H), 1.39(s, 9H), 1.44-1.82(brm, 12H), 2.36(m, 1H), 3.22(s, 3H), 3.30(m, 3H), 3.51(s, 1H), 4.19(m, 2H), 5.04(m, 1H), 7.06(d, 1H), 7.24(1H), 7.37(d, 2H), 7.51(d, 2H) MS APCl m/z 577 [MH]⁺ |
| 83 | propyl | 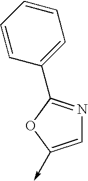 | ethyl | ¹HNMR (CDCl₃, 400MHz): 0.82(t, 3H), 1.44(s, 9H), 1.40-1.71(m, 12H), 1.80(d, 1H), 2.01(m, 4H), 2.04(m, 1H), 3.09(dd, 1H), 3.21(dd, 1H), 4.17(m, 2H), 4.86(m, 1H), 7.36(m, 1H), 7.43(m, 3H), 7.56(m, 1H), 8.01(m, 2H) MS ES+ m/z 549 [MNa]⁺ |
| 84 | methoxyethyl | 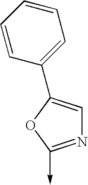 | isobutyl | ¹HNMR (CDCl₃, 400MHz): 0.88(m, 6H), 1.40(s, 9H), 1.47-2.10(brm, (14H), 3.23(s, 3H), 3.27(m, 2H), 3.48(m, 1H), 3.91(m, 3H), 5.06(m, 1H), 7.11(d, 1H) 7.22-7.72(brm, 6H) MS APCl m/z 571 [MH]⁺ |
| 85 | methoxyethyl | 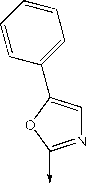 | isopropyl | ¹HNMR (CDCl₃, 400MHz): 1.16(dd, 3H), 1.19(dd, 3H), 1.38(s, 9H), 1.43(m, 2H), 1.61(m, 6H), 2.02(m, 4H), 2.31(m, 1H), 3.20(s, 3H), 3.27(m, 4H), 3.43(m, 7.08(d,1H), 7.21 (s, 1H), 5.02(m, 1H), 7.31(m, 1H), 2H), 7.57(m, |
| # MS ES+ m/z 579 [MNa]⁺ | | | | |
| 86 | methoxyethyl | 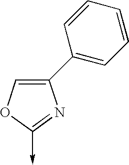 | ethyl | ¹HNMR (CDCl₃, 400MHz): 0.84(m, 3H), 1.38(s, 9H), 1.37-1.51 (brm, 2H), 1.59-1.84(brm, 6H), 2.08(m, 4H), 2.37(m, 1H), 3.45(s, 3H), 3.44(m, 4H), 4.17(m, 2H), 5.03(m, 1H) 7.27(m, 2H), 7.38(m, 2H), 7.66(m, 2H), 7.82(s, 1H) |
| # MS ES+ m/z 1108 [M₂Na]⁺ | | | | |
| 87 | methoxyethyl | 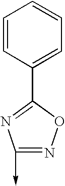 | methyl | ¹HNMR (CDCl₃, 400MHz): 1.40(s, 9H), 1.49-1.72(m, 10H), 2.07(m, 2H), 2.37(m, 1H), 3.23(s, 3H), 3.32(m, 3H), 3.44(m, 1H), 3.76(m, 3H), 5.06(m, 1H), 6.98(d, 1H), 7.56(m, 2H), 7.60(m, 1H), 8.09(m, 2H) |
| 88 | methoxyethyl | 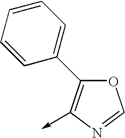 | ethyl | ¹HNMR (CDCl₃, 400MHz): 0.82(t, 3H), 1.08(t, 3H), 1.23(m, 2H), 1.31 (m, 1H), 1.42(s, 9H), 1.58(m, 7H), 1.79(m, 1H), 1.97(m, 3H), 2.22(m, 1H), 3.19(dd, 1H), 3.51(dd, 1H), 3.97(m, 1H), 4.05(m, 1H), 4.93(m, 1H), 7.42(m, 4H), 7.61(m, 2H), |
| # 7.86(m, 1H) MS APCl m/z 527 [MH]⁺ | | | | |
| 89 | methoxyethyl | 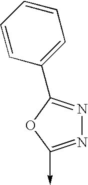 | ethyl | ¹HNMR (CDCl₃, 400MHz): 1.22(t, 3H), 1.38(s, 9H), 1.42-1.81(m, 9H), 2.03(m, 3H), 2.36(m, 1H), 3.21(s, 3H), 3.31(m, 2H), 3.46(dd, 1H), 3.58(dd, 1H), 4.23(m, 2H), 5.09(m, 1H), 6.94(d, 1H), 7.53(m, 3H), 8.04(m, 2H) |
| # MS ES+ m/z 1109 [M₂Na]⁺ | | | | |
| 90 | propyl | 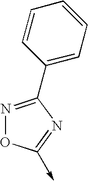 | benzyl | ¹HNMR (CDCl₃, 400MHz): 0.82(t, 3H), 1.17-1.78(m, 20H), 2.05(m, 3H), 2.19(m, 1H), 3.49(m, 2H), 3.63(m, 1H), 5.17(m, 3H), 7.07(d, 2H), 7.24(m, 3H), 7.51 (m, 3H), 8.00(m, 2H) MS ES+ m/z 612 [MNa]⁺ |
| 91 | propyl | 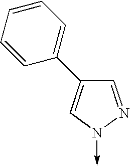 | benzyl | ¹HNMR (CDCl₃, 400MHz): 0.84(t, 3H), 1.17-1.77(m,20H), 2.02(m, 3H), 2.27(m, 1H), 4.52(dd, 1H), 4.70(dd, 1H), 4.97(m, 1H), 5.14(m, 2H), 7.08(d, 1H), 7.19-7.46(m, 10H), 7.75(m, 2H) MS ES+ m/z 610 [MNa]⁺ |
| 92 | methoxyethyl | 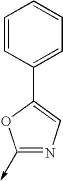 | butoxyethyl | ¹HNMR (CDCl₃, 400MHz): 0.87(t, 3H), 1.16-1.36(m, 3H), 1.39(s, 9H), 1.49(m, 4H), 1.59-1.73(m, 4H), 1.78(m, 2H), 2.03(m, 3H), 2.33(m, 1H), 3.21(s, 3H), 3.32(m, 2H), 3.38(m, 3H), 3.57(m, 3H), 4.29(m, 2H), 5.12(m, 1H), |
| # 7.36(m, 1H), 7.42(m, 2H), 7.59(m, 2H), 7.64(m, 1H) MS ES+ m/z 637 [MNa]⁺ | | | | |
| 93 | methoxyethyl | 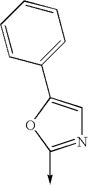 | cyclopentyl | ¹HNMR (CDCl₃, 400MHz): 1.40(s, 9H), 1.51-1.82(m, 16H), 2.03(m, 4H), 2.32(m, 1H), 3.24(s, 3H), 3.31(m, 3H), 3.57(m, 1H), 4.98(m, 1H), 5.19(m, 1H), 7.15(d, 1H), 7.26(m, 2H), 7.41(m, 2H), 7.59(m, 2H) MS ES+ m/z 605 [MNa]⁺ |
| 94 | methoxyethyl | 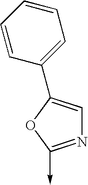 | 3-pentyl | ¹HNMR (CDCl₃, 400MHz): 0.80(t, 3H), 0.85(t, 3H), 1.34(s, 9H), 1.47-1.81(m, 13H), 2.06(m, 3H), 2.32(m, 1H), 3.22(s, 3H), 3.28(m, 2H), 3.40(dd, 1H), 3.58(dd, 1H), 4.79(m, 1H), 5.07(m, 1H), 7.24(m, 3H), 7.41(m, 2H), 7.60(m, 2H) MS ES+ m/z 607 |
| # [MNa]⁺ | | | | |
| 95 | methoxyethyl | 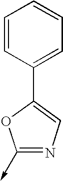 | benzyl | ¹HNMR(DMSOD₆, 400MHz): 1.31(s, 9H), 1.37-1.64(m, 7H), 1.80(m, 3H), 1.96(m, 2H), 2.14(m, 1H), 3.09(m, 1H), 3.29(m, 3H), 3.39(m, 1H), 4.00(m, 1H), 4.86(m, 1H), 5.08(m, 2H), 5.58(m, 1H) 7.30(m, 5H), 7.42(m, 2H), 7.53(m, 2H), 7.62(m, 2H), 8.12(m, 1H) |
| # MS ES+ m/z 627 [MNa]⁺ | | | | |
| 96 | propyl | 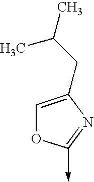 | benzyl | ¹HNMR(CDCl₃, 400MHz): 0.83(m, 3H), 0.89(m, 6H), 1.19-1.36(m, 3H), 1.39(s, 9H), 1.42-1.66(m, 7H), 1.78(m, 1H), 1.83-2.05(m, 4H), 2.13(m, 3H), 3.17(dd, 1H), 3.39(dd, 1H), 5.02(m, 1H), 5.09(m, 2H), 7.19-7.39(m, 6H) MS ES+ m/z 569 [MH]⁺ |
| 97 | propyl | 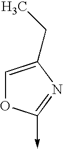 | benzyl | ¹HNMR(CDCl₃, 400MHz): 0.74(t, 3H), 1.11(t, 3H), 1.20-1.98(m, 23H), 2.18(m, 3H), 2.39(m, 2H), 3.13(dd, 1H), 3.34(dd, 1H), 4.97(m, 1H), 5.08(m, 2H), 7.16(m, 1H), 7.21-7.33(m, 5H) MS ES+ m/z 541 [MH]⁺ |
| 98 | propyl | 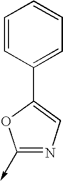 | propyl | MS ES+ m/z 563 [MNa]⁺ |
| 99 | propyl | 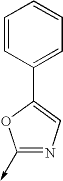 | butyl | MS ES+ m/z 577 [MNa]⁺ |

¹After stirring for 18 hours the reaction mixture was concentrated in vacuo and purified by column chroatorgraphy as described.

Preparation 100

(2R)-2-{1-[(1S)-1-Ethoxycarbonylmethyl-2-(5-phenyl-oxazol-2-yl)-ethylcarbamoyl]-cyclopentylmethyl}-pentanoic Acid Tert-butyl Ester

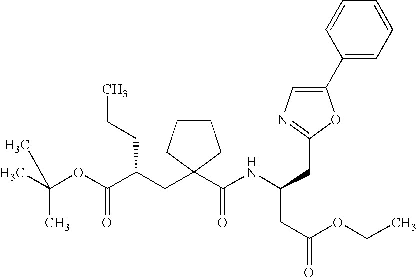

The title compound was prepared with 1-[(2R)-2-(tert-butoxycarbonyl-pentyl)]-cyclopentanecarboxylic acid (WO 0202513 preparation 2) and the amine of preparation 72 by the method described in preparation 76, except that the reaction mixture was diluted with dichloromethane (50 mL) rather than ethyl acetate; [α]=−16.73; ¹HNMR (CDCl₃, 400 MHz): 0.82 (t, 3H), 1.22 (m, 3H), 1.37 (s, 9H), 1.40-1.96 (m, 14H), 2.17 (m, 1H), 2.58 (dd, 1H), 2.63 (dd, 1H), 3.14 (dd, 1H), 3.21 (dd, 1H), 4.10 (m, 2H), 4.78 (m, 1H), 6.94 (m, 1H), 7.17 (m, 1H), 7.23 (m, 1H), 7.39 (m, 2H), 7.59 (m, 2H); MS ES+ m/z 563 [MNa]⁺.

The following compounds of the general formula shown below were also prepared by the method described above using the appropriate acid and amine.

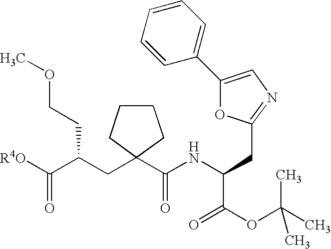

| Ex. No. | R₄ | Data |
|---|---|---|
| 101 | propyl | ¹HNMR(CDCl₃, 400MHz): 0.81(m, 3H), 1.39(s, |
| | | 9H), 1.42-1.92(m, 14H), 2.44(m, 1H), 3.25(s, |
| | | 3H), 3.28(m, 2H), 3.49(m, 2H), 3.98(m, 2H), |
| | | 5.05(m, 1H) 6.94(m, 1H), 7.22(m, 1H), 7.37(m, |
| | | 1H), 7.42(m, 2H), 7.59(m, 2H); MS ES+ m/z 579 |
| | | [MNa]⁺ |
| 102 | butyl | ¹HNMR(CDCl₃, 400MHz): 0.86(m, 3H), 1.40(s, |
| | | 9H), 1.43-1.92(m, 14H), 2.02(m, 3H), 2.44(m, |
| | | 1H), 3.25(s, 3H), 3.27(m, 2H), 3.41(m, 1H), |
| | | 4.01(m, 2H), 4.89(m, 1H), 6.96(m, 1H), 7.20(m, |
| | | 1H), 7.28(m, 1H), 7.41(m, 2H), 7.60(m, 2H); MS |
| | | ES+ m/z 593 [MNa]⁺ |

Preparation 103

(2R)-2-{1-[(1S)-1-Ethoxycarbonyl-2-(5-phenyloxazol-2-yl)-ethylcarbamoyl]-cyclopentylmethyl}-pentanoic Acid Tert-butyl Ester

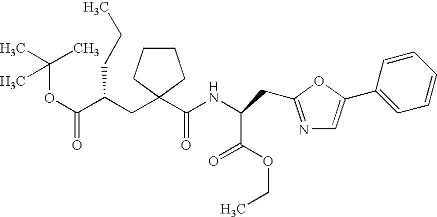

The amine hydrochloride of preparation 52 (260 mg, 1 mmol) was dissolved in water (15 mL) and washed with ethyl acetate (2×10 mL). The aqueous was basified with saturated potassium carbonate solution and extracted with ethyl acetate (3×20 mL). The organics were combined, dried over magnesium sulphate and concentrated in vacuo. The product was added to a solution of 1-[(2R)-2-(tert-butoxycarbonyl-pentyl)]-cyclopentanecarboxylic acid (WO 0202513 preparation 2) (284 mg, 1 mmol), 1-(3-dimethylaminopropyl)-3-ethylcarbodiimide hydrochloride (293 mg, 1.5 mmol), 1-hydroxybenzotriazole hydrate (205 mg, 1.5 mmol) and 4-methylmorpholine (434 μL, 4 mmol) in dichloromethane (7 mL) and the reaction mixture stirred at room temperature for 18 hours. The crude product was purified by column chromatography on silica gel, eluting with pentane:ethyl acetate (83:17 to 66:33) to yield the title product, 120 mg; ¹HNMR (CDCl₃, 400 MHz): 0.82 (m, 3H), 1.22 (m, 3H), 1.40 (s, 9H), 1.57-2.41 (brm, 14H), 3.61 (m, 2H), 4.18 (m, 2H), 5.06 (d, 1H), 7.05 (d, 1H), 7.18-7.63 (brm, 6H); MS ES+ m/z 549 [MNa]⁺.

Preparation 104

(2S)-2-{1-[(1S)-1-benzyloxycarbonyl-2-(3-phenyl-[1,2,4]oxadiazol-5-yl)-ethylcarbamoyl]-cyclopentylmethyl}-4-methoxy-butyric Acid Ethyl Ester

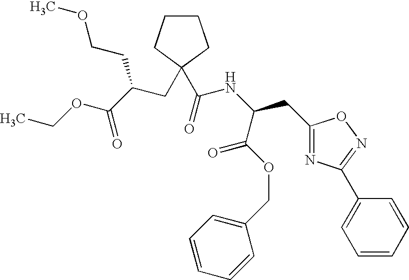

The carboxylic acid of preparation 11 (156 mg, 0.57 mmol) was added to a solution of O-(1H benzotriazol-1-yl)-N,N,N′,N′-tetramethyluronium tetrafluoroborate (275 mg, 0.85 mmol), 1-hydroxybenzotriazole hydrate (116 mg, 0.858 mmol) and N-ethyldiisopropylamine (294 μL, 1.72 mmol) in dichloromethane (5 mL) and the reaction mixture stirred for 5 minutes. The amine of preparation 61 (275 mg, 0.63 mmol) was added and the reaction mixture stirred at room temperature for 18 hours. N,N-Dimethylformamide (200 μL) and additional N-ethyldiisopropylamine (200 μL) were added and the reaction mixture stirred at room temperature for 18 further hours. The reaction mixture was concentrated in vacuo and the crude product purified by column chromatography on silica gel eluting with pentane:ethyl acetate (100:0 to 95:5 to 90:10) to yield the title product co-eluted with a by-product. Appropriate fractions were concentrated in vacuo, dissolved in ether (50 mL), washed with 2M hydrochloric acid (20 mL), saturated sodium hydrogencarbonate solution (20 mL) and brine (20 mL), dried over magnesium sulphate and concentrated in vacuo to yield the title product, 100 mg; ¹HNMR (CDCl₃, 400 MHz): 1.17 (t, 3H), 1.58-1.79 (m, 9H), 2.06 (m, 3H), 2.44 (m, 1H), 3.20 (s, 3H), 3.25 (m, 2H), 3.48 (dd, 1H), 3.63 (dd, 1H), 3.99 (m, 2H), 5.16 (m, 2H), 7.06 (d, 1H), 7.24 (m, 5H), 7.51 (m, 3H), 7.98 (m, 2H); MS ES+ m/z 600 [MNa]⁺.

Preparation 105

(2S)-2-{1-[(1S)-1-Carboxy-2-(4-phenyl-imidazol-1-yl)-ethylcarbamoyl]-cyclopentylmethyl}-4-methoxy-butyric Acid Tert Butyl Ester

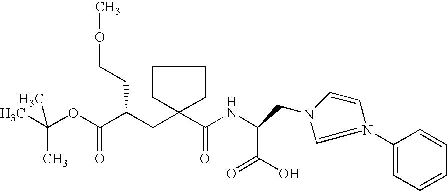

1M sodium hydroxide (1 mL) was added to a solution of the di-ester of preparation 107 (47 mg, 0.087 mmol) in dioxan (3 mL) and the reaction mixture stirred at room temperature for 18 hours. The reaction mixture was concentrated in vacuo and partitioned between water (20 mL) and ethyl acetate (20 mL). The aqueous layer was acidified with glacial acetic acid and extracted with ethyl acetate (2×20 mL). The organics were combined, dried over magnesium sulphate and concentrated in vacuo. The product was azeotroped with toluene (3×20 mL), ethyl acetate (20 mL) and dichloromethane (20 mL) to yield the title product, 19 mg; ¹HNMR (CDCl₃, 400 MHz): 1.23 (m, 3H), 1.43 (s, 9H), 1.50-1.87 (m, 8H), 2.07 (m, 3H), 3.26 (s, 3H), 3.41 (m, 2H), 4.54-4.86 (m, 3H), 5.02 (m, 1H), 6.94 (m, 1H), 7.31 (m, 3H), 7.71-7.86 (m, 2H), 8.62 (m, 1H); MS ES− m/z 512 [MH]⁻.

Preparation 106

(2S)-2-{1-[(1S)-1Carboxy-2-(3-phenyl-[1,2,4]oxadiazol-5-yl)-ethylcarbamoyl]-cyclopentylmethyl}-4-methoxy-butyric Acid Ethyl Ester

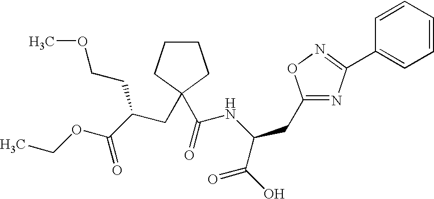

The di-ester of preparation 104 (90 mg) was added to a suspension of 10% Pd/C (10 mg) in ethanol (5 mL) and the reaction mixture stirred at room temperature under 50 psi and hydrogen for 2 hours. The reaction mixture was filtered through Arbocel®, eluting with ethanol and the filtrate concentrated in vacuo. The crude product was purified by column chromatography on silica gel eluting with pentane:ethyl acetate (70:30). The product of this was heated in 10% potassium carbonate solution and treated with pentane:ethyl acetate 90:10 solution to yield a precipitate. The mixture was filtered and the aqueous layer separated before being acidified with 2M hydrochloric acid and extracted into ethyl acetate (10 mL). The organic layer was dried over magnesium sulphate and concentrated in vacuo to yield the title product, 6 mg; ¹HNMR (CDCl₃, 400 MHz): 1.19 (m, 3H), 1.40-1.76 (m, 7H), 1.81 (m, 2H), 2.08 (m, 3H), 2.47 (m, 1H), 3.23 (s, 3H), 3.51 (m, 2H), 3.63 (m, 2H), 4.02 (m, 2H), 5.04 (m, 1H), 7.25 (m, 2H), 7.46 (m, 2H), 8.03 (m, 1H); MS ES+ m/z 488 [MH]⁺.

Preparation 107

(2S)-2-{1-[(1S)-1-Ethoxycarbonyl-2-(4-phenyl-imidazol-1-yl)-ethylcarbamoyl]-cyclopentylmethyl}-4-methoxy Butyric Acid Tert Butyl Ester

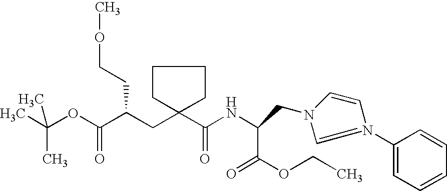

The amine of preparation 64 (19 mg, 0.074 mmol) was added to a solution of 1-[(2S)-2-(tert-butoxycarbonyl)-4-methoxybutyl]cyclopentanecarboxylic acid (WO 0279143 page 71 preparation 69) (22 mg, 0.074 mmol), 1-(3-dimethylaminopropyl)-3-ethylcarbodiimide hydrochloride (15.4 mg, 0.082 mmol), 1-hydroxybenzotriazole hydrate (11 mg, 0.082 mmol) and 4-methylmorpholine (42 mg, 0.37 mmol) in dichloromethane (5 mL) and the reaction mixture stirred at room temperature for 18 hours. The reaction mixture was poured into water (50 mL) and extracted with dichloromethane (2×50 mL). The combined organics were dried over sodium sulphate and concentrated in vacuo. The crude product was purified by column chromatography on silica gel eluting with pentane:ethyl acetate 99:1 to 95:5 to yield the title product, 20 mg; ¹HNMR(CDCl₃, 400 MHz): 1.32 (t, 3H), 1.47 (s, 9H), 1.49-1.88 (m, 9H), 2.02 (m, 3H), 2.22 (m, 1H), 3.27 (s, 3H), 3.36 (m, 2H), 4.24 (m, 2H), 4.41 (dd, 1H), 4.55 (dd, 1H), 4.87 (m, 1H), 6.42 (m, 1H), 7.22 (m, 1H), 7.37 (m, 3H), 7.66 (m, 1H), 7.78 (m, 2H); MS ES+ m/z 564 [MNa]⁺.

Biological Assays

Assay 1: IC₅₀ values of the compounds of the invention against NEP and ACE were determined using methods described in published patent application EP1097719-A1, paragraphs [0368] to [0376]. The IC₅₀ values presented below were determined using NEP (EC.3.4.24.11) from human kidney.

Assay 2: This is a modification of Assay 1 which is adapted for potent tight-binding inhibitors.

Materials

Buffer: 50 mM Tris pH 7.4

Buffer containing 4% DMSO: 4 ml DMSO added to 96 ml buffer

Enzyme: NEP from human kidney (prepared using the method of Kenny and Booth, Biochem. J. 142, 575, 1974) aliquots stored at −70° C. The NEP activity of each preparation is determined by using serial dilutions of the preparation in this assay without inhibitor, a dilution that achieves 10 to 15% conversion of substrate is used in inhibitor studies.

Inhibitors: Inhibitors are dissolved in DMSO to give a 10 mM solution then further diluted to 1 mM in DMSO. A further dilution is made (40 μl into 960 μl buffer) to give 4×10⁻⁵M inhibitor in buffer containing 4% DMSO. All further dilutions are made in buffer containing 4% DMSO. Inhibitors are assayed utilising an eight point (half log units) dose response curve in duplicate. From this data the IC₅₀ is calculated.

Substrate: The substrate (Abz-D-Arg-Arg-Leu-EDDnp, K. M. Carvalho et al. Analytical Biochem 237, 167-173 (1996)) is stored at −70° C. A 2 mM stock is made in buffer and aliquots stored at −70° C. for up to 1 month.

Stop solution: Phosphoramidon (Sigma R7385) 300 nM in buffer. Aliquots of 300 μM solution are stored at −20° C. and diluted 1000 fold in buffer.

Product: Two samples corresponding to 100% conversion of substrate to product are included in the assay to enable the % substrate turnover to be determined. Add 200 μl of 25 μM product and 100 μl stop solution. A stock of this is generated by incubating 1 ml of 25 μM substrate with 20 μl of enzyme stock overnight at 37° C., aliquot and store at −70° C.

Equipment

Plates: Black Costar 96 well plate Cat# 3915

Fluorescence reader: BMG Fluostar, read at ex320 em420

Method

1. Add 50 μl of inhibitor (4× assay conc)

2. Add 80 μl of buffer

3. Add 50 μl of enzyme dilution (1:800)

4. Incubate at 37° C. for 1 hour

5. Initiate the reaction by adding 20 μl of substrate

5. Incubate at 37° C. for 3 hours

6. Add 100 μl of stop solution.

7. Read fluorescence using a FLUOstar. (Ex320/Em420

Included in each assay are:

Non enzyme blanks (enzyme replaced with buffer and compound dilution replaced with 4% DMSO buffer)

Controls (compound dilution replaced with 4% DMSO buffer)

Total product (see below)

The compounds of the invention are inhibitors of NEP.

The title compounds of the Examples are potent inhibitors of NEP.

The title compounds of Examples 25-27, 29-31, 34-36, 39 and 40 showed an IC₅₀ against NEP of less than or equal to 10 nM as determined by Assay 1.

As determined by Assay 2, the title compound of Example 26 showed an IC₅₀ against NEP of 0.3 nM and a selectivity over ACE of greater than 100 fold; the title compound of Example 29 showed an IC₅₀ against NEP of 0.7 nM and a selectivity over ACE of greater than 100 fold; the title compound of Example 30 showed an IC₅₀ against NEP of 0.3 nM and a selectivity over ACE of greater than 100 fold; and the title compound of Example 40 showed an IC₅₀ against NEP of 0.3 nM and a selectivity over ACE of greater than 100 fold.

1. A compound of formula (I), a pharmaceutically acceptable salt or solvate thereof 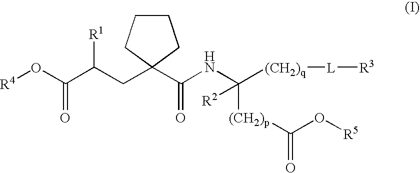
wherein
R¹ is C₁-C₆alkyl, C₁-C₆alkoxyC₁-C₃alkyl or C₁-C₆alkoxyC₁-C₆alkoxyC₁-C₃alkyl;
R² is hydrogen or C₁-C₆alkyl;
L is an aromatic heterocyclic ring, optionally substituted with C₁-C₆alkyl or halo;
R³ is C₁-C₆alkyl optionally substituted by halo, alkoxy, haloalkoxy, alkylthio, haloalkylthio or nitrile group, or R³ is phenyl or aromatic heterocyclyl each of which may be independently substituted by one or more alkyl, halo, haloalkyl, alkoxy, haloalkoxy, alkylthio, haloalkylthio or nitrile group;
R⁴ and R⁵ are either both hydrogen, or one of R⁴ and R⁵ is hydrogen and the other is a biolabile ester-forming group that in the body of a patient is replaced by hydrogen;
p is 0, 1 or 2; and
q is 1 or 2.

2. A compound according to claim 1 wherein R¹ is C₁-C₆alkyl or C₁-C₆alkoxyC₁-C₃alkyl.

3. A compound according to claim 2 wherein R¹ is propyl or methoxyethyl.

4. A compound according to claim 1 wherein R² is hydrogen.

5. A compound according to claim 3 wherein R² is hydrogen.

6. A compound according to claim 1 wherein L is a non-fused aromatic heterocyclic ring optionally substituted by C₁-C₆alkyl.

7. A compound according to claim 5 wherein L is a non-fused aromatic heterocyclic ring optionally substituted by C₁-C₆alkyl.

8. A compound according to claim 1 wherein L is a five membered aromatic heterocyclic ring.

9. A compound according to claim 7 wherein L is a five membered aromatic heterocyclic ring.

10. A compound according to claim 9 wherein L is oxazole, oxadiazole, imidazole or pyrazole.

11. A compound according to claim 10 wherein L is oxazole or oxadiazole.

12. A compound according to claim 1 wherein R³ is C₁-C₆alkyl or R³ is phenyl which may be independently substituted by one or more C₁-C₆alkyl, halo, haloC₁-C₆alkyl, C₁-C₆alkoxy, haloalkoxy, C₁-C₆alkylthio, haloC₁-C₆alkylthio or nitrile group.

13. A compound according to claim 10 wherein R³ is C₁-C₆alkyl or R³ is phenyl which may be independently substituted by one or more C₁-C₆alkyl, halo, haloC₁-C₆alkyl, C₁-C₆alkoxy, haloalkoxy, C₁-C₆alkylthio, haloC₁-C₆alkylthio or nitrile group.

14. A compound according to claim 12 wherein R³ is phenyl optionally substituted by halo.

15. A compound according to claim 14 wherein R³ is phenyl, 4-fluorophenyl, or 4-chlorophenyl.

16. A compound according to claim 13 wherein R³ is phenyl, 4-fluorophenyl, or 4-chlorophenyl.

17. A compound according to claim 1 wherein the biolabile ester-forming groups are C₁-C₆alkyl, carbocyclyl or heterocyclyl each of which may be substituted.

18. A compound according to claim 17 wherein the biolabile ester-forming groups is:
i) C₁-C₆alkyl optionally substituted by hydroxy, oxo, halo, haloC₁-C₆alkyl, C₁-C₆alkoxy, haloC₁-C₆alkoxy, C₁-C₆alkylthio, haloC₁-C₆alkylthio, nitrile, carbocyclyl, heterocyclyl, carbocyclyloxy, heterocyclyloxy, C₁-C₇alkylcarbonyloxy, carbocyclylcarbonyloxy, heterocyclylcarbonyloxy, alkylcarbonylamino, and alkylaminocarbonyl, wherein any carbocyclyl or heterocyclyl group is optionally substituted by C₁-C₆alkyl, halo, haloC₁-C₆alkyl, C₁-C₆alkoxy, haloC₁-C₆alkoxy, C₁-C₆alkylthio, haloC₁-C₆alkylthio or nitrile; or
ii) carbocyclyl or heterocyclyl optionally substituted by C₁-C₆alkyl, halo, haloC₁-C₆alkyl, C₁-C₆alkoxy, haloC₁-C₆alkoxy, C₁-C₆alkylthio, haloC₁-C₆alkylthio or nitrile.

19. A compound according to claim 18 wherein any carbocyclic group is phenyl and any heterocyclic group is aromatic.

20. A compound according to claim 18 wherein the biolabile ester-forming group is: ethyl, propyl, butyl, isobutyl, cyclopentyl, benzyl, 1-(2,2-diethylbutyryloxy)ethyl, 2-ethylpropionyloxymethyl, 1-(2-ethylpropionyloxy)ethyl, 1-(2,4-dimethylbenzoyloxy)ethyl, 1-benzoyloxy)benzyl, 1-(benzoyloxy)ethyl, 2-methyl-1-propionyloxypropyl, 2,4,6-trimethylbenzoyloxymethyl, 1-(2,4,6-trimethylbenzyloxy)ethyl, pivaloyloxymethyl, phenethyl, phenpropyl, 2,2,2-trifluororethyl, 1-naphthyl, 2-naphthyl, 2,4-dimethylphenyl, 4-t-butylphenyl, 5-(4-methyl-1,3-dioxalynyl-2-onyl)methyl, N,N-diethylaminocarbonylmethyl or 5-indanyl.

21. A compound according to claim 1 wherein R⁴ and R⁵ are both hydrogen.

22. A compound according to claim 13 wherein R⁴ and R⁵ are both hydrogen.

23. A compound according to claim 16 wherein R⁴ and R⁵ are both hydrogen.

24. A compound according to claim 1 wherein p is 0 or 1.

25. A compound according to claim 23 wherein p is 0 or 1.

26. A compound according to claim 1 wherein q is 1.

27. A compound according to claim 25 wherein q is 1.

28. A compound according to claim 1 wherein the compound is of formula (I′) 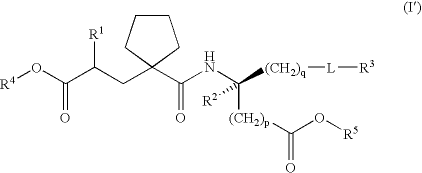
and R¹, R², R³, R⁴, R⁵, L, p, and q are as defined in claim 1.

29. A compound according to claim 28 wherein R¹ is C₁-C₆alkyl or C₁-C₆alkoxyC₁-C₃alkyl.

30. A compound according to claim 29 wherein R¹ is propyl or methoxyethyl.

31. A compound according to claim 28 wherein R² is hydrogen.

32. A compound according to claim 30 wherein R² is hydrogen.

33. A compound according to claim 28 wherein L is a non-fused aromatic heterocyclic ring optionally substituted by C₁-C₆alkyl.

34. A compound according to claim 32 wherein L is a non-fused aromatic heterocyclic ring optionally substituted by C₁-C₆alkyl.

35. A compound according to claim 28 wherein L is a five membered aromatic heterocyclic ring.

36. A compound according to claim 7 wherein 34 is a five membered aromatic heterocyclic ring.

37. A compound according to claim 34 wherein L is oxazole, oxadiazole, imidazole or pyrazole.

38. A compound according to claim 37 wherein L is oxazole or oxadiazole.

39. A compound according to claim 28 wherein R³ is C₁-C₆alkyl or R³ is phenyl which may be independently substituted by one or more C₁-C₆alkyl, halo, haloC₁-C₆alkyl, C₁-C₆alkoxy, haloalkoxy, C₁-C₆alkylthio, haloC₁-C₆alkylthio or nitrile group.

40. A compound according to claim 37 wherein R³ is C₁-C₆alkyl or R³ is phenyl which may be independently substituted by one or more C₁-C₆alkyl, halo, haloC₁-C₆alkyl, C₁-C₆alkoxy, haloalkoxy, C₁-C₆alkylthio, haloC₁-C₆alkylthio or nitrile group.

41. A compound according to claim 39 wherein R³ is phenyl optionally substituted by halo.

42. A compound according to claim 41 wherein R³ is phenyl, 4-fluorophenyl, or 4-chlorophenyl.

43. A compound according to claim 40 wherein R³ is phenyl, 4-fluorophenyl, or 4-chlorophenyl.

44. A compound according to claim 28 wherein the biolabile ester-forming groups are C₁-C₆alkyl, carbocyclyl or heterocyclyl each of which may be substituted.

45. A compound according to claim 44 wherein the biolabile ester-forming groups is:
i) C₁-C₆alkyl optionally substituted by hydroxy, oxo, halo, haloC₁-C₆alkyl, C₁-C₆alkoxy, haloC₁-C₆alkoxy, C₁-C₆alkylthio, haloC₁-C₆alkylthio, nitrile, carbocyclyl, heterocyclyl, carbocyclyloxy, heterocyclyloxy, C₁-C₇alkylcarbonyloxy, carbocyclylcarbonyloxy, heterocyclylcarbonyloxy, alkylcarbonylamino, and alkylaminocarbonyl, wherein any carbocyclyl or heterocyclyl group is optionally substituted by C₁-C₆alkyl, halo, haloC₁-C₆alkyl, C₁-C₆alkoxy, haloC₁-C₆alkoxy, C₁-C₆alkylthio, haloC₁-C₆alkylthio or nitrile; or
ii) carbocyclyl or heterocyclyl optionally substituted by C₁-C₆alkyl, halo, haloC₁-C₆alkyl, C₁-C₆alkoxy, haloC₁-C₆alkoxy, C₁-C₆alkylthio, haloC₁-C₆alkylthio or nitrile.

46. A compound according to claim 45 wherein any carbocyclic group is phenyl and any heterocyclic group is aromatic.

47. A compound according to claim 45 wherein the biolabile ester-forming group is: ethyl, propyl, butyl, isobutyl, cyclopentyl, benzyl, 1-(2,2-diethylbutyryloxy)ethyl, 2-ethylpropionyloxymethyl, 1-(2-ethylpropionyloxy)ethyl, 1-(2,4-dimethylbenzoyloxy)ethyl, 1-benzoyloxy)benzyl, 1-(benzoyloxy)ethyl, 2-methyl-1-propionyloxypropyl, 2,4,6-trimethylbenzoyloxymethyl, 1-(2,4,6-trimethylbenzyloxy)ethyl, pivaloyloxymethyl, phenethyl, phenpropyl, 2,2,2-trifluororethyl, 1-naphthyl, 2-naphthyl, 2,4-dimethylphenyl, 4-t-butylphenyl, 5-(4-methyl-1,3-dioxalynyl-2-onyl)methyl, N,N-diethylaminocarbonylmethyl or 5-indanyl.

48. A compound according to claim 28 wherein R⁴ and R⁵ are both hydrogen.

49. A compound according to claim 40 wherein R⁴ and R⁵ are both hydrogen.

50. A compound according to claim 43 wherein R⁴ and R⁵ are both hydrogen.

51. A compound according to claim 28 wherein p is 0 or 1.

52. A compound according to claim 50 wherein p is 0 or 1.

53. A compound according to claim 28 wherein q is 1.

54. A compound according to claim 52 wherein q is 1.

55. A compound according to claim 28 wherein
R¹ is C₁-C₆alkyl or C₁-C₆alkoxyC₁-C₃alkyl;
R² is hydrogen;
L is a non-fused five membered aromatic heterocyclic ring optionally substituted by C₁-C₆alkyl;
R³ is C₁-C₆alkyl or R³ is phenyl which may be independently substituted by one or more C₁-C₆alkyl, halo, haloC₁-C₆alkyl, C₁-C₆alkoxy, haloalkoxy, C₁-C₆alkylthio, haloC₁-C₆alkylthio or nitrile group;
R⁴ and R⁵ are either both hydrogen, or one of R⁴ and R⁵ is hydrogen and the other is a biolabile ester-forming group selected from:
i) C₁-C₆alkyl optionally substituted by hydroxy, oxo, halo, haloC₁-C₆alkyl, C₁-C₆alkoxy, haloC₁-C₆alkoxy, C₁-C₆alkylthio, haloC₁-C₆alkylthio, nitrile, carbocyclyl, heterocyclyl, carbocyclyloxy, heterocyclyloxy, alkylcarbonyloxy, carbocyclylcarbonyloxy, heterocyclylcarbonyloxy, alkylcarbonylamino, or alkylaminocarbonyl, wherein any carbocyclyl or heterocyclyl group is optionally substituted by C₁-C₆alkyl, halo, haloC₁-C₆alkyl, C₁-C₆alkoxy, haloC₁-C₆alkoxy, C₁-C₆alkylthio, haloC₁-C₆alkylthio or nitrile; or
ii) carbocyclyl or heterocyclyl optionally substituted by C₁-C₆alkyl, halo, haloC₁-C₆alkyl, C₁-C₆alkoxy, haloC₁-C₆alkoxy, C₁-C₆alkylthio, haloC₁-C₆alkylthio or nitrile;
p is 0 or 1; and
q is 1.

56. A compound according to claim 28 wherein
R¹ is propyl or methoxyethyl;
R² is hydrogen;
L is oxazole, oxadiazole, imidazole or pyrazole each of which may be substituted by C₁-C₆alkyl;
R³ is phenyl, 4-fluorophenyl, or 4-chlorophenyl;
R⁴ and R⁵ are both hydrogen;
p is 0; and
q is 1.

57. A compound according to claim 1 selected from:
(2S)-2-{1-[(1S)-1-Carboxy-2-(4-methyl-5-phenyl-oxazol-2-yl)-ethoxycarbamoyl]-cyclopentylmethyl}-4-methoxy-butyric acid;
(2S)-2-(1-{(1S)-1-Carboxy-2-[5-(4-fluoro-phenyl)-oxazol-2-yl]-ethylcarbamoyl}-cyclopentylmethyl-4-methoxy-butyric acid;
(2R)-2-{1-[(1S)-1-Carboxy-2-(5-phenyl-oxazol-2-yl)-ethylcarbamoyl]-cyclopentylmethyl}-pentanoic acid;
(2S)-2-(1-{(1S)-1-Carboxy-2-[5-(4-chloro-phenyl)-oxazol-2-yl]-ethylcarbamoyl}-cyclopentylmethyl)-4-methoxy-butyric acid;
(2S)-2-{1-[(1S)-1-Carboxy-2-(5-phenyl-[1.2.4]oxadiazol-3-yl)-ethylcarbamoyl]-cyclopentlymethyl}-4-methoxy-butyric acid;
(2R)-2-{1-[(1S)-1-carboxy-2-(4-phenyl-pyrazol-1-yl)-ethylcarbamoyl]-cyclopentylmethyl}-pentanoic acid; and
(2S)-2-{1-[(1S)-1-Carboxy-2-(5-phenyl-oxazol-2-yl)-ethylcarbamoyl]-cyclopentylmethyl}-4-methoxy-butyric acid.

58. A method of treating or preventing a condition for which a beneficial response is obtained by the inhibition of neutral endopeptidase in a mammal comprising treating said mammal with a therapeutically effective amount of a compound defined in any one of claims 1 to 57, a pharmaceutically acceptable salt or solvate thereof.

59. The method according to claim 58 wherein the condition is a cardiovascular disease or condition.

60. The method according to claim 59 wherein the condition is hypertension.

61. The method according to claim 58 wherein the condition is female sexual dysfunction or male erectile dysfunction.

62. A pharmaceutical composition comprising a compound defined in any one of claims 1 to 57, a pharmaceutically acceptable salt, solvate, polymorph or prodrug thereof together with a pharmaceutically acceptable excipient, diluent or carrier.

63. A pharmaceutical composition comprising a compound defined in any one of claims 1 to 57, a pharmaceutically acceptable salt, solvate, polymorph or prodrug thereof and one or more:
a) angiotensin receptor blockers;
b) calcium channel blockers;
c) statins;
d) PDE5 inhibitors;
e) beta blockers;
f) ACE inhibitors;
g) alpha-blockers;
h) selective aldosterone receptor antagonists;
i) imidazoline I₁ agonists; or
j) endothelin receptor antagonists or endothelin converting enzyme inhibitors.

64. The pharmaceutical composition of claim 63 wherein
a) the angiotensin receptor blocker is losartan, valsartan, telmisartan, candesartan, irbesartan, eprosartan or olmesartan;
b) the calcium channel blocker is amlodipine;
c) the statin is atorvastatin;
d) the PDE5 inhibitor is sildenafil, tadalafil, vardenafil, 5-[2-ethoxy-5-(4-ethylpiperazin-1-ylsulphonyl)pyridin-3-yl]-3-ethyl-2-[2-methoxyethyl]-2,6-dihydro-7H-pyrazolo[4,3-d]pyrimidin-7-one, 5-(5-acetyl-2-butoxy-3-pyridinyl)-3-ethyl-2-(1-ethyl-3-azetidinyl)-2,6-dihydro-7H-pyrazolo[4,3-d]pyrimidin-7-one, or N-[[3-(4,7-dihydro-1-methyl-7-oxo-3-propyl-1H-pyrazolo[4,3-d]-pyrimidin-5-yl)-4-propxyphenyl]sulfonyl]-1-methyl2-pyrrolidinepropanamide;
e) the beta blocker is atenolol or carvedilol;
f) the ACE inhibitor is quinapril, enalapril or lisinopril;
g) the alpha-blocker is doxazosin;
h) the selective aldosterone receptor antagonist is eplerenone or spironolactone; and
i) the imidazoline I₁ agonist is rilmenidine.
